SEQUENCE LISTING (41 sequences)

SEQ ID NO: 1 - protein, 336 aa, Homo sapiens
ILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIDYLEIDNRQCVQASSQCFQSATDVAAFLGALASLGSLNIPYKIEAVQSETVEPPPPAQLHFMYVAAAAFVLLFFVGCGVLLSRKRRR

SEQ ID NO: 2 - protein, 326 aa, Mus sp.
ILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIDYLEIDNRQCVQSSSQCFQSATDVAAFLGALASLGSLNIPYKIEAVKSEPVEPPLPSQLHLMYVAAAAFVLLFFVGCGVLLSRKRRR

SEQ ID NO: 3 - protein, 336 aa, Homo sapiens
ILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRR

SEQ ID NO: 4 - protein, 326 aa, Mus sp.
ILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRR

SEQ ID NO: 5 - protein, 295 aa, Mus sp.
LYTAPTSTPPATCQSQYCADKARDGICDEACNSHACQWDGGDCSLTMEDPWANCTSTLRCWEYINNQCDEQCNTAECLFDNFECQRNSKTCKYDKYCADHFKDNHCDQGCNSEECGWDGLDCASDQPENLAEGTLIIVVLLPPEQLLQDSRSFLRALGTLLHTNLRIKQDSQGALMVYPYFGEKSAAMKKQKMTRRSLPEEQEQEQEVIGSKIFLEIDNRQCVQDSDQCFKNTDAAAALLASHAIQGTLSYPLVSVFSELESPRNAQLLYLLAVAVVIILFFILLGVIMAKRKRK

SEQ ID NO: 6 - protein, 297 aa, Homo sapiens
APAAAPEVSEEPRCPRAACQAKRGDQRCDRECNSPGCGWDGGDCSLSVGDPWRQCEALQCWRLFNNSRCDPACSSPACLYDNFDCHAGGRERTCNPVYEKYCADHFADGRCDQGCNTEECGWDGLDCASEVPALLARGVLVLTVLLPPEELLRSSADFLQRLSAILRTSLRFRLDAHGQAMVFPYHRPSPGSEPRARRELAPEVIGSVVMLEIDNRLCLQSPENDHCFPDAQSAADYLGALSAVERLDFPYPLRDVRGEPLEPPEPSVPLLPLLVAGAVLLLVILVLGVMVARRKRE

SEQ ID NO: 7 - protein, 244 aa, synthetic construct
EVQLVESGGGLVQPGGSLRLSCAASGFTFSSYWIHWVRQAPGKGLEWVARINPPNRSNQYADSVKGRFTISADTSKNTAYLQMNSLRAEDTAVYYCARGSGFRWVMDYWGQGTLVTVSSGGSSRSSSSGGGGSGGGGDIQMTQSPSSLSASVGDRVTITCRASQDVSTAVAWYQQKPGKAPKLLIYSASFLYSGVPSRFSGSGSGTDFTLTISSLQPEDFATYYCQQFYTTPSTFGQGTKVEIK

SEQ ID NO: 8 - protein, 244 aa, synthetic construct
EVQLVESGGGLVQPGGSLRLSCAASGFTFSSYWIHWVRQAPGKGLEWVARINPSNGSTNYADSVKGRFTISADTSKNTAYLQMNSLRAEDTAVYYCARGSGFRWVMDYWGQGTLVTVSSGGSSRSSSSGGGGSGGGGDIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIK

SEQ ID NO: 9 - protein, 244 aa, synthetic construct
EVQLVESGGGLVQPGGSLRLSCAASGFTFSSYWIHWVRQAPGKGLEWVARINPANGSTRYADSVKGRFTISADTSKNTAYLQMNSLRAEDTAVYYCARGSGFRWVMDYWGQGTLVTVSSGGSSRSSSSGGGGSGGGGDIQMTQSPSSLSASVGDRVTITCRASQDVSTAVAWYQQKPGKAPKLLIYSASFLYSGVPSRFSGSGSGTDFTLTISSLQPEDFATYYCQQSFSTPATFGQGTKVEIK

SEQ ID NO: 10 - protein, 248 aa, synthetic construct
QVQLVQSGAEVKKPGASVKISCKVSGYTLRGYWIEWVRQAPGKGLEWIGQILPGTGRTNYNEKFKGRVTMTADTSTDTAYMELSSLRSEDTAVYYCARFDGNYGYYAMDYWGQGTTVTVSSGGSSRSSSSGGGGSGGGGQAVVTQEPSLTVSPGGTVTLTCRSSTGAVTTSNYANWFQQKPGQAPRTLIGGTNNRAPGVPARFSGSLLGGKAALTLSGAQPEDEAEYYCALWYSNHWVFGGGTKLTVL

SEQ ID NO: 11 - protein, 248 aa, synthetic construct
EVQLVESGGGLVQPGGSLRLSCAASGYTFSSYGMSWVRQAPGKGLEWVSYIYPYSGATYYADSVKGRFTISADTSKNTAYLQMNSLRAEDTAVYYCARHSGYYRISSAMDVWGQGTLVTVSAGGSSRSSSSGGGGSGGGGDIQMTQSPSSLSASVGDRVTITCRASQNIKRFLAWYQQKPGKAPKLLIYGASTRESGVPSRFSGSGSGTDFTLTISSLQPEDFATYYCQQYYRSPHTFGQGTKVEIKR

SEQ ID NO: 12 - protein, 244 aa, synthetic construct
EVQLVESGGGLVQPGGSLRLSCAASGYAFTDYWMTWVRQAPGKGLEWVAEISPNSGGTNFNEKFKGRFTISVDNAKNSLYLQMNSLRAEDTAVYYCARGEIRYNWFAYWGQGTLVTVSSGGSSRSSSSGGGGSGGGGDIQMTQSPSSLSASVGDRVTITCKASQNVGNNIAWYQQKPGKAPKLLIYYASNRYTGVPSRFSGSGYGTDFTLTISSLQPEDFATYYCQRLYNSPFTFGGGTKVEIK

SEQ ID NO: 13 - protein, 8 aa, synthetic construct
VGCGVLLS

SEQ ID NO: 14 - protein, 7 aa, synthetic construct
GCGVLLS

SEQ ID NO: 15 - protein, 22 aa, synthetic construct
HLMYVAAAAFVLLFFVGCGVLL

SEQ ID NO: 16 - protein, 56 aa, synthetic construct
IPYKIEAVKSEPVEPPLPSQLHLMYVAAAAFVLLFFVGCGVLLSRKRRRQLCIQKL

SEQ ID NO: 17 - protein, 6 aa, synthetic construct
  VARIANT 2: note = Leucine or Glutamine
PXGMTS

SEQ ID NO: 18 - protein, 5 aa, synthetic construct
  VARIANT 2: note = Leucine or Glutamine
PXGMT

SEQ ID NO: 19 - protein, 38 aa, synthetic construct
PVEPPLPSQLHLMYVAAAAFVLLFFVGCGVLLSRKRRR

SEQ ID NO: 20 - protein, 45 aa, synthetic construct
GSNSGGTLEGGGSGENLYFQGGGGGRTGGGSEQKLISEEDLGGGS

SEQ ID NO: 21 - protein, 35 aa, synthetic construct
LEGGGSGENLYFQSGRTGGGSEQKLISEEDLGGGS

SEQ ID NO: 22 - protein, 16 aa, synthetic construct
GGGSGENLYFQGGGGS

SEQ ID NO: 23 - protein, 17 aa, synthetic construct
GGGSGDYKDDDDKGGGS

SEQ ID NO: 24 - protein, 14 aa, synthetic construct
GGGSGIEGRGGGGS

SEQ ID NO: 25 - protein, 30 aa, synthetic construct
LEGGGSGPLGLAGGRTADYKDDDDKGGGSG

SEQ ID NO: 26 - protein, 30 aa, synthetic construct
LEGGGSGLALGPGGRTADYKDDDDKGGGSG

SEQ ID NO: 27 - protein, 40 aa, synthetic construct
LEGGSGGTVRPAHLRDSGGGSGGTRTADYKDDDDKGGGSG

SEQ ID NO: 28 - protein, 38 aa, synthetic construct
LEGGGSGYIHPFHLVIHNESGGRTADYKDDDDKGGGSG

SEQ ID NO: 29 - protein, 924 aa, synthetic construct
MALPVTALLLPLALLLHAARPYPYDVPDYATGMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGGGSTGDGGGGEVQLVESGGGLVQPGGSLRLSCAASGFTFSSYWIHWVRQAPGKGLEWVARINPPNRSNQYADSVKGRFTISADTSKNTAYLQMNSLRAEDTAVYYCARGSGFRWVMDYWGQGTLVTVSSGGSSRSSSSGGGGSGGGGDIQMTQSPSSLSASVGDRVTITCRASQDVSTAVAWYQQKPGKAPKLLIYSASFLYSGVPSRFSGSGSGTDFTLTISSLQPEDFATYYCQQFYTTPSTFGQGTKVEIKGSNSGGTLEGGGSGENLYFQGGGGGRTGGGSEQKLISEEDLGGGSILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIDYLEIDNRQCVQSSSQCFQSATDVAAFLGALASLGSLNIPYKIEAVKSEPVEGPV

SEQ ID NO: 30 - protein, 887 aa, synthetic construct
MALPVTALLLPLALLLHAARPYPYDVPDYATGMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGGGSTGDGGGGEVQLVESGGGLVQPGGSLRLSCAASGFTFSSYWIHWVRQAPGKGLEWVARINPPNRSNQYADSVKGRFTISADTSKNTAYLQMNSLRAEDTAVYYCARGSGFRWVMDYWGQGTLVTVSSGGSSRSSSSGGGGSGGGGDIQMTQSPSSLSASVGDRVTITCRASQDVSTAVAWYQQKPGKAPKLLIYSASFLYSGVPSRFSGSGSGTDFTLTISSLQPEDFATYYCQQFYTTPSTFGQGTKVEIKGSNSGGTLEGGGSGENLYFQGGGGGRTGGGSEQKLISEEDLGGGSILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGMDIRGSIDYLEIDNRQCVQSSSQCFQSATDVAAFLGALASLGSLNIPYKIEAVKSEPVEGPV

SEQ ID NO: 31 - protein, 924 aa, synthetic construct
MALPVTALLLPLALLLHAARPYPYDVPDYATGMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGGGSTGDGGGGEVQLVESGGGLVQPGGSLRLSCAASGFTFSSYWIHWVRQAPGKGLEWVARINPPNRSNQYADSVKGRFTISADTSKNTAYLQMNSLRAEDTAVYYCARGSGFRWVMDYWGQGTLVTVSSGGSSRSSSSGGGGSGGGGDIQMTQSPSSLSASVGDRVTITCRASQDVSTAVAWYQQKPGKAPKLLIYSASFLYSGVPSRFSGSGSGTDFTLTISSLQPEDFATYYCQQFYTTPSTFGQGTKVEIKGSNSGGTLEGGGSGENLYFQGGGGGRTGGGSEQKLISEEDLGGGSILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEGPV

SEQ ID NO: 32 - protein, 954 aa, synthetic construct
MALPVTALLLPLALLLHAARPYPYDVPDYATGMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGGGSTGDGGGGQVQLVQSGAEVKKPGASVKISCKVSGYTLRGYWIEWVRQAPGKGLEWIGQILPGTGRTNYNEKFKGRVTMTADTSTDTAYMELSSLRSEDTAVYYCARFDGNYGYYAMDYWGQGTTVTVSSGGSSRSSSSGGGGSGGGGQAVVTQEPSLTVSPGGTVTLTCRSSTGAVTTSNYANWFQQKPGQAPRTLIGGTNNRAPGVPARFSGSLLGGKAALTLSGAQPEDEAEYYCALWYSNHWVFGGGTKLTVLGSNSGGTLEGGGSGENLYFQGGGGGRTGGGSEQKLISEEDLGGGSILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPGSLVPRGSSHHHHHHGPV

SEQ ID NO: 33 - protein, 899 aa, synthetic construct
MALPVTALLLPLALLLHAARPYPYDVPDYATGMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGGGSTGDGGGGEVQLVESGGGLVQPGGSLRLSCAASGYTFSSYGMSWVRQAPGKGLEWVSYIYPYSGATYYADSVKGRFTISADTSKNTAYLQMNSLRAEDTAVYYCARHSGYYRISSAMDVWGQGTLVTVSAGGSSRSSSSGGGGSGGGGDIQMTQSPSSLSASVGDRVTITCRASQNIKRFLAWYQQKPGKAPKLLIYGASTRESGVPSRFSGSGSGTDFTLTISSLQPEDFATYYCQQYYRSPHTFGQGTKVEIKRGSNSGGTLEGGGSGENLYFQGGGGGRTGGGSEQKLISEEDLGGGSLYTAPTSTPPATCQSQYCADKARDGICDEACNSHACQWDGGDCSLTMEDPWANCTSTLRCWEYINNQCDEQCNTAECLFDNFECQRNSKTCKYDKYCADHFKDNHCDQGCNSEECGWDGLDCASDQPENLAEGTLIIVVLLPPEQLLQDSRSFLRALGTLLHTNLRIKQDSQGALMVYPYFGEKSAAMKKQKMTRRSLPEEQEQEQEVIGSKIFLEIDNRQCVQDSDQCFKNTDAAAALLASHAIQGTLSYPLVSVFSEPVEGPV

SEQ ID NO: 34 - protein, 895 aa, synthetic construct
MALPVTALLLPLALLLHAARPYPYDVPDYATGMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGGGSTGDGGGGEVQLVESGGGLVQPGGSLRLSCAASGYAFTDYWMTWVRQAPGKGLEWVAEISPNSGGTNFNEKFKGRFTISVDNAKNSLYLQMNSLRAEDTAVYYCARGEIRYNWFAYWGQGTLVTVSSGGSSRSSSSGGGGSGGGGDIQMTQSPSSLSASVGDRVTITCKASQNVGNNIAWYQQKPGKAPKLLIYYASNRYTGVPSRFSGSGYGTDFTLTISSLQPEDFATYYCQRLYNSPFTFGGGTKVEIKGSNSGGTLEGGGSGENLYFQGGGGGRTGGGSEQKLISEEDLGGGSAPAAAPEVSEEPRCPRAACQAKRGDQRCDRECNSPGCGWDGGDCSLSVGDPWRQCEALQCWRLFNNSRCDPACSSPACLYDNFDCHAGGRERTCNPVYEKYCADHFADGRCDQGCNTEECGWDGLDCASEVPALLARGVLVLTVLLPPEELLRSSADFLQRLSAILRTSLRFRLDAHGQAMVFPYHRPSPGSEPRARRELAPEVIGSVVMLEIDNRLCLQSPENDHCFPDAQSAADYLGALSAVERLDFPYPLRDVRGEPVEGPV

SEQ ID NO: 35 - protein, 1420 aa, synthetic construct
MALPVTALLLPLALLLHAARPYPYDVPDYATGMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGGGSTGDGGGGEVQLVESGGGLVQPGGSLRLSCAASGFTFSSYWIHWVRQAPGKGLEWVARINPPNRSNQYADSVKGRFTISADTSKNTAYLQMNSLRAEDTAVYYCARGSGFRWVMDYWGQGTLVTVSSGGSSRSSSSGGGGSGGGGDIQMTQSPSSLSASVGDRVTITCRASQDVSTAVAWYQQKPGKAPKLLIYSASFLYSGVPSRFSGSGSGTDFTLTISSLQPEDFATYYCQQFYTTPSTFGQGTKVEIKGSNSGGTLEGGGSGENLYFQGGGGGRTGGGSEQKLISEEDLGGGSILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIDYLEIDNRQCVQSSSQCFQSATDVAAFLGALASLGSLNIPYKIEAVKSEPVEPPLPSQLHLMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGSMEGRGSLLTCGDVEENPGPGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 36 - protein, 1420 aa, synthetic construct
MALPVTALLLPLALLLHAARPYPYDVPDYATGMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGGGSTGDGGGGEVQLVESGGGLVQPGGSLRLSCAASGFTFSSYWIHWVRQAPGKGLEWVARINPPNRSNQYADSVKGRFTISADTSKNTAYLQMNSLRAEDTAVYYCARGSGFRWVMDYWGQGTLVTVSSGGSSRSSSSGGGGSGGGGDIQMTQSPSSLSASVGDRVTITCRASQDVSTAVAWYQQKPGKAPKLLIYSASFLYSGVPSRFSGSGSGTDFTLTISSLQPEDFATYYCQQFYTTPSTFGQGTKVEIKLEGGGSGENLYFQSGRTGGGSEQKLISEEDLGGGSILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIDYLEIDNRQCVQASSQCFQSATDVAAFLGALASLGSLNIPYKIEAVQSETVEPPPPAQLHFMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGSMEGRGSLLTCGDVEENPGPGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 37 - protein, 1465 aa, synthetic construct
MALPVTALLLPLALLLHAARPYPYDVPDYATGMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGGGSTGDGGGGEVQLVESGGGLVQPGGSLRLSCAASGFTFSSYWIHWVRQAPGKGLEWVARINPPNRSNQYADSVKGRFTISADTSKNTAYLQMNSLRAEDTAVYYCARGSGFRWVMDYWGQGTLVTVSSGGSSRSSSSGGGGSGGGGDIQMTQSPSSLSASVGDRVTITCRASQDVSTAVAWYQQKPGKAPKLLIYSASFLYSGVPSRFSGSGSGTDFTLTISSLQPEDFATYYCQQFYTTPSTFGQGTKVEIKGSNSGGTLEGGGSGENLYFQGGGGGRTGGGSEQKLISEEDLGGGSILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIDYLEIDNRQCVQSSSQCFQSATDVAAFLGALASLGSLNIPYKIEAVKSEPVEPPLPSQLHLMYVAAAAFVLLFFVGCGVLLSRKRRRQLCIQKLMSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPGSMEGRGSLLTCGDVEENPGPGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 38 - protein, 20 aa, synthetic construct
LEGGGSENLYFQSGRTGGGS

SEQ ID NO: 39 - protein, 7 aa, synthetic construct
ENLYFQS

SEQ ID NO: 40 - protein, 6 aa, synthetic construct
HHHHHH

SEQ ID NO: 41 - protein, 18 aa, synthetic construct
GGGSEQKLISEEDLGGGS

(19) United States
(12) Patent Application Publication
(10) Pub. No.: US 2025/0289885 A1
(43) Pub. Date: Sep. 18, 2025

(54) ENGINEERED PROTEASE ACTIVITY RESPONSIVE RECEPTORS AND USES THEREOF

(71) Applicant: TRUSTEES OF BOSTON UNIVERSITY, Boston, MA (US)

(72) Inventors: John Tuan NGO, Cambridge, MA (US); Jeremy Christian Francois TRAN, Brookline, MA (US)

(73) Assignee: TRUSTEES OF BOSTON UNIVERSITY, Boston, MA (US)

(21) Appl. No.: 19/080,346

(22) Filed: Mar. 14, 2025

Related U.S. Application Data
(60) Provisional application No. 63/565,681, filed on Mar. 15, 2024.

(51) Int. Cl. C07K 16/28; C07K 14/705

(52) U.S. Cl. CPC C07K 16/28; C07K 14/705; C07K 2317/92; C07K 2319/035; C07K 2319/50

(57) ABSTRACT

Described herein are compositions, methods, and systems for modulating Notch receptor activation. Aspects of the invention relate to synthetic proteins comprising at least a mutant Notch NRR (Negative Regulatory Region). Another aspect of the invention relates to a protease-dependent synthetic protein. Engineered cells comprising the synthetic protein are additionally described herein.

### CROSS-REFERENCE TO RELATED APPLICATION

This application claims benefit under 35 U.S.C. § 119(e) of the U.S. Provisional Application No. 63/565,681 filed Mar. 15, 2024, the contents of each of which are incorporated herein by reference in their entireties.

### GOVERNMENT SUPPORT

This invention was made with government support under GM128859 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Apr. 8, 2025, is named 701586-000129USPT_SL.xml and is 50,523 bytes in size.

### TECHNICAL FIELD

The present invention relates generally to compositions and methods for engineering regulatable natural and synthetic Notch signaling.

### BACKGROUND OF THE INVENTION

Drug-inducible strategies for regulating protein function and gene activity have been indispensable tools in biological research, yet methods for controlling diverse systems remain lacking. The Notch protein is a transmembrane receptor that acts a mechanical “switch,” translating mechanical cues into gene expression. This mechanosensing activity is achieved via Notch's force-sensitive Negative Regulatory Region (NRR), which contains three LNR domains. In the resting state, the LNR domains adopt an autoinhibitory conformation that sterically hinders proteolytic cleavage necessary for receptor activation. Upon the application of a pulling force, however, these LNR domains are displaced, and two concomitant proteolytic cleavages occur that release the Notch intracellular domain to transport to the nucleus and regulate gene expression.

Synthetic receptors disclosed herein fill a gap in the mammalian synthetic receptor toolkit in sensing extracellular proteolysis with customizable input-output relationships. This allows for an additional layer of sophistication in constructing synthetic signaling networks for higher specificity by integrated this receptor with other synthetic receptors. The generalized nature of the receptor enables user-defined proteolysis to control many processes due to its modular intracellular domain (ICD). Furthermore, data presented herein demonstrate that the soluble form of this autoinhibited domain can be used to develop protease-conditional Notch agonists. These molecules have potential therapeutic applications in diseases where hyperactive Notch signaling and aberrant proteolysis occur, such as cancer.

### SUMMARY OF THE INVENTION

As described herein, compositions, methods, and systems using antibody domains have been developed through which signaling from natural and synthetic Notch receptors can be regulated via cleavable linkers.

One aspect provided herein describes a synthetic nucleic acid encoding a mutant Notch NRR (Negative Regulatory Region), wherein the mutant Notch NRR comprises a mutation that induces ligand-independent activation of regulated intramembrane proteolysis resulting to Notch signaling, SynNotch signaling, or at least another signaling outcome.

In one embodiment of this aspect, or any aspect herein, the mutant Notch NRR comprises at least point mutation as compared to a wild-type Notch NRR.

In one embodiment of this aspect, or any aspect herein, the mutant Notch NRR comprises a V1667D mutation as compared to a wild-type Notch NRR, or the mutant Notch NRR comprises a mutation selected from Table 4.

In one embodiment of this aspect, or any aspect herein, the mutant Notch NRR sequence encodes a protein sequence of SEQ ID NO: 1 or 2.

One aspect provided herein describes a mutant Notch NRR polypeptide encoded any of the nucleic acids disclosed herein.

In one embodiment of this aspect, or any aspect herein, the mutant Notch NRR protein comprises a sequence of SEQ ID NO: 1 or 2.

One aspect provided herein describes a fusion protein comprising: a Notch NRR (Negative Regulatory Region)-binding antibody, and a mutant Notch NRR comprising a mutation that induces ligand-independent activation of regulated intramembrane proteolysis resulting to Notch signaling, SynNotch signaling, or at least another signaling outcome.

In one embodiment of this aspect, or any aspect herein, the Notch NRR-binding antibody inhibits the ligand-independent activation of Notch signaling by the mutant Notch NRR.

In one embodiment of this aspect, or any aspect herein, the Notch NRR-binding antibody is a Notch NRR-binding scFv, Notch NRR-binding nanobody, or Notch NRR-binding scFab.

In one embodiment of this aspect, or any aspect herein, the Notch NRR-binding scFv is an anti-NRR1 scFv.

In one embodiment of this aspect, or any aspect herein, the NRR1 comprises a of SEQ ID NO: 3 or 4.

In one embodiment of this aspect, or any aspect herein, the mutant Notch NRR comprises at least point mutation as compared to a wild-type Notch NRR.

In one embodiment of this aspect, or any aspect herein, the mutant Notch NRR comprises a V1667D mutation as compared to a wild-type Notch NRR.

In one embodiment of this aspect, or any aspect herein, the mutant Notch NRR protein comprises a sequence of SEQ ID NO: 1 or 2.

In one embodiment of this aspect, or any aspect herein, the Notch NRR-binding immunoglobulin element is further fused to a transmembrane domain.

In one embodiment of this aspect, or any aspect herein, the fusion protein further comprises, positioned in between i) and ii), at least one linker.

In one embodiment of this aspect, or any aspect herein, the Notch NRR-binding immunoglobulin element further comprises at lease one linker.

In one embodiment of this aspect, or any aspect herein, the Notch NRR-binding scFv further comprises at least one linker positioned between the heavy and light chains.

In one embodiment of this aspect, or any aspect herein, the at least one linker is a cleavable linker.

In one embodiment of this aspect, or any aspect herein, the cleavable linker is cleaved by a protease, an enzyme, chemical reagent, light, pH, or ultrasound.

In one embodiment of this aspect, or any aspect herein, the at least one linker is cleaved by a protease selected from the group consisting of: Matrix Metalloproteinases (MMPs), Urokinase, aka urokinase-type plasminogen activator (uPA), Tobacco Etch Virus (TEV) protease, Enterokinase (enteropeptidase), Prostate-Specific Antigen (PSA), Cathepsins, ADAM-family proteases, ADAM-TSs, Kallikreins, Legumain, Fibroblast activating protein-α (FAP), Renin, Human Rhinovirus (HRV) 3C protease, Bacterial sortases, Thrombin, and Type II Transmembrane Serine Proteases.

In one embodiment of this aspect, or any aspect herein, the transmembrane domain comprises the human Notch1 transmembrane domain.

In one embodiment of this aspect, or any aspect herein, the Notch NRR-binding scFv comprises, from N-terminal to C-terminal and in covalent linkage, a V_{H} domain, a linker domain, and a V_{L} domain.

In one embodiment of this aspect, or any aspect herein, the scFv is selected from any one of SEQ ID NOs: 7-12.

In one embodiment of this aspect, or any aspect herein, the fusion protein further comprising a signal sequence at its N-terminal.

One aspect provided herein describes a fusion protein comprising: a Notch NRR (Negative Regulatory Region)-binding scFv, at lease one linker, and a mutant Notch NRR, mutant Notch NRR comprises a mutation that induces ligand-independent activation of regulated intramembrane proteolysis resulting to Notch signaling, SynNotch signaling, or at least another signaling outcome.

One aspect provided herein describes a nucleic acid sequence encoding any of the fusion proteins disclosed herein.

In one embodiment of this aspect, or any aspect herein, the synthetic nucleic acid isolated.

One aspect provided herein describes a synthetic Notch receptor protein comprising, from N-terminal to C-terminal and in covalent linkage, (i) a scFv that binds to an at least one Notch NRR, (ii) at least one linker, (iii) a mutated Notch NRR, wherein the mutant Notch NRR comprises a mutation that induces ligand-independent activation of regulated intramembrane proteolysis resulting to Notch signaling, SynNotch signaling, or at least another signaling outcome, (iv) a transmembrane domain, and (v) an intracellular domain.

In one embodiment of this aspect, or any aspect herein, the mutant Notch NRR comprises a V1667D mutation as compared to a wild-type Notch NRR.

In one embodiment of this aspect, or any aspect herein, the mutant Notch NRR has a sequence of SEQ ID NO: 1 or 2.

In one embodiment of this aspect, or any aspect herein, the at least one linker is a cleavable linker.

In one embodiment of this aspect, or any aspect herein, the cleavable linker is cleaved by a protease, an enzyme, chemical reagent, light, pH or ultrasound.

In one embodiment of this aspect, or any aspect herein, the at least one linker is cleaved by a protease selected from the group consisting of: Matrix Metalloproteinases (MMPs), Urokinase, aka urokinase-type plasminogen activator (uPA), Tobacco Etch Virus (TEV) protease, Enterokinase (enteropeptidase), Prostate-Specific Antigen (PSA), Cathepsins, ADAM-family proteases, ADAM-TSs, Kallikreins, Legumain, Fibroblast activating protein-α (FAP), Renin, Human Rhinovirus (HRV) 3C protease, Bacterial sortases, Thrombin, and Type II Transmembrane Serine Proteases

In one embodiment of this aspect, or any aspect herein, the transmembrane domain comprises the human Notch1 transmembrane domain.

In one embodiment of this aspect, or any aspect herein, the scFv comprises, from N-terminal to C-terminal and in covalent linkage, a V_{H} domain, a linker domain, and a V_{L} domain.

In one embodiment of this aspect, or any aspect herein, the scFv is selected from any one of SEQ ID NOs: 7-12.

In one embodiment of this aspect, or any aspect herein, the scFV further comprising a signal sequence N-terminal to the scFv.

One aspect provided herein describes an isolated nucleic acid sequence encoding the synthetic protein disclosed herein.

One aspect provided herein describes an engineered cell comprising the isolated nucleic acid sequence disclosed herein.

In one embodiment of this aspect, or any aspect herein, the engineered cell is an engineered T cell, an engineered stem cell, an engineered innate immune cell, or an engineered natural killer cell.

One aspect provided herein describes an engineered cell comprising (i) a nucleic acid sequence encoding a Notch NRR-binding antibody, (ii) at least one linker, and (iii) a nucleic acid sequence encoding a synthetic Notch receptor protein comprising a mutated Notch NRR, wherein the mutant Notch NRR comprises a mutation that induces ligand-independent activation of regulated intramembrane proteolysis resulting to Notch signaling, SynNotch signaling, or at least another signaling outcome.

T In one embodiment of this aspect, or any aspect herein, the engineered cell is an engineered T cell, an engineered stem cell, an engineered innate immune cell, or an engineered natural killer cell.

In one embodiment of this aspect, or any aspect herein, the at least one linker is a cleavable linker.

One aspect provided herein describes a protease regulatable Notch signaling modulator, the modulator comprising an anti-NRR antibody fused to a mutant NRR, having at least one protease cleavable linker positioned between the anti-NRR immunoglobulin element and mutant NRR.

In one embodiment of this aspect, or any aspect herein, the mutant NRR is an NRR from Notch1, Notch2, or Notch3.

In one embodiment of this aspect, or any aspect herein, the anti-NRR antibody is an anti-NRR scFv.

In one embodiment of this aspect, or any aspect herein, the anti-NRR scFv binds to NRR1, NRR2, NRR3.

In one embodiment of this aspect, or any aspect herein, the at least one protease cleavable linker is recognized by TEV, an MMP, other cancer-associated proteolytic enzymes, a chemical reagent, light, acidic pH or ultrasound.

In one embodiment of this aspect, or any aspect herein, further comprising at least one AND-gate proteins.

In one embodiment of this aspect, or any aspect herein, the at least one AND-gate protein provides independent and/or distinct cleavage sites required to make the antigen binding site of the anti-NRR antibody available.

In one embodiment of this aspect, or any aspect herein, the mutant NRR domain comprises a S1 furin cleavage loop.

In one embodiment of this aspect, or any aspect herein, the mutant NRR domain lacks a S1 furin cleavage loop.

In one embodiment of this aspect, or any aspect herein, anti-NRR immunoglobulin element is a NRR-binding agonist capable of activating Notch receptors upon cleavage and separation from the fused mutant NRR.

In one embodiment of this aspect, or any aspect herein, the modulation is an increase in Notch signaling.

In one embodiment of this aspect, or any aspect herein, the modulation is a decrease in Notch signaling.

One aspect provided herein describes a protease regulatable Notch signaling modulator, the inhibitor comprising an anti-NRR antibody fused to a transmembrane domain, and a mutant NRR, having at least one protease cleavable linker positioned between the anti-NRR immunoglobulin element fused to the transmembrane domain and mutant NRR.

In one embodiment of this aspect, or any aspect herein, cleavage of the at least one protease cleavable linker result in dissociation of the mutant NRR and the anti-NRR immunoglobulin element fused to a transmembrane domain.

In one embodiment of this aspect, or any aspect herein, dissociation results in inhibition of Notch signaling via binding of the anti-NRR antibody fused to a transmembrane domain to a receptor.

In one embodiment of this aspect, or any aspect herein, modulation is an increase in Notch signaling.

In one embodiment of this aspect, or any aspect herein, modulation is a decrease in Notch signaling.

One aspect provided herein describes a gate protein-synthetic Notch receptor comprising: a signal sequence, a ligand binding domain (such as a tumor antigen), a NRR domain; a cleavable linker; a NRR-binding scFv; a Notch1 TMD; and an intracellular domain

In one embodiment of this aspect, or any aspect herein, the ICD is an ICD derived from Gal4, TetR, zinc fingers, Cas9, Cre recombinase, the Notch ICD (NICD), a dominant negative MAML (dnMAML), an inhibitor of transcriptional activity, and a protein with a nucleus-specific activity.

In one embodiment of this aspect, or any aspect herein, the modulator is further conjugated to a drug, forming an antibody-drug conjugate.

In one embodiment of this aspect, or any aspect herein, the modulator is used to deliver cellular cargo.

### Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology, and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

The term “antibody” broadly refers to any immunoglobulin (Ig) molecule and immunologically active portions of immunoglobulin molecules (i.e., molecules that contain an antigen binding site that immunospecifically bind an antigen) comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. Nonlimiting embodiments of which are discussed below, and include but are not limited to a variety of forms, including full length antibodies and antigen-binding portions thereof; including, for example, an immunoglobulin molecule, a monoclonal antibody, a chimeric antibody, a CDR-grafted antibody, a human antibody, a humanized antibody, a single chain antibody, a Fab, a F(ab′), a F(ab′)2, a Fv antibody, fragments produced by a Fab expression library, a disulfide linked Fv, a scFv, a single domain antibody (dAb), a diabody, a multispecific antibody, a dual specific antibody, an anti-idiotypic antibody, a bispecific antibody, a functionally active epitope-binding fragment thereof, bifunctional hybrid antibodies (e.g., Lanzavecchia et al., Eur. J. Immunol. 17, 105 (1987)) and single chains (e.g., Huston et al., Proc. Natl. Acad. Sci. U.S.A., 85, 5879-5883 (1988) and Bird et al., Science 242, 423-426 (1988), which are incorporated herein by reference) and/or antigen-binding fragments of any of the above (See, generally, Hood et al., Immunology, Benjamin, N.Y., 2ND ed. (1984), Harlow and Lane, Antibodies. A Laboratory Manual, Cold Spring Harbor Laboratory (1988) and Hunkapiller and Hood, Nature, 323, 15-16 (1986), which are incorporated herein by reference). Antibodies also refer to immunoglobulin molecules and immunologically active portions of immunoglobulin molecules, i.e., molecules that contain antigen or target binding sites or “antigen-binding fragments.” The antibody or immunoglobulin molecules described herein can be of any type (e.g., IgG, IgE, IgM, IgD, IgA and IgY), class (e.g., IgG1, IgG2, IgG3, IgG4, IgA1 and IgA2) or subclass of immunoglobulin molecule, as is understood by one of skill in the art. Furthermore, in humans, the light chain can be a kappa chain or a lambda chain.

In a full-length antibody, each heavy chain is comprised of a heavy chain variable domain (abbreviated herein as HCVR or VH) and a heavy chain constant region. The heavy chain constant region is comprised of three domains: CH1, CH2, and CH3. Each light chain is comprised of a light chain variable domain (abbreviated herein LCVR as VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. This structure is well-known to those skilled in the art. The chains are usually linked to one another via disulfide bonds.

The term “Fc region” is used to define the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, a CH2 domain, and a CH3 domain, and optionally comprises a CH4 domain. Replacements of amino acid residues in the Fc portion to alter antibody effector function are known in the art (U.S. Pat. Nos. 5,648,260 and 5,624,821). The Fc portion of an antibody mediates several important effector functions, for example, cytokine induction, ADCC, phagocytosis, complement dependent cytotoxicity (CDC), and half-life/clearance rate of antibody and antigen-antibody complexes. In some cases these effector functions are desirable for therapeutic antibody but in other cases might be unnecessary or even deleterious, depending on the therapeutic objectives. Certain human IgG isotypes, particularly IgG1 and IgG3, mediate ADCC and CDC via binding to Fc.gamma.Rs and complement Clq, respectively. Neonatal Fc receptors (FcRn) are the critical components determining the circulating half-life of antibodies

The term “antigen-binding portion” of an antibody refers to one or more fragments of an antibody that retain the ability to specifically bind to an antigen. Antigen-binding functions of an antibody can be performed by fragments of a full-length antibody. Such antibody fragment embodiments may also be incorporated in bispecific, dual specific, or multi-specific formats such as a dual variable domain (DVD-Ig) format; specifically binding to two or more different antigens (e.g., Notch receptor and a different antigen molecule). Examples of binding fragments encompassed within the term “antigen-binding portion” of an antibody include (i) a Fab fragment, a monovalent fragment consisting of the VL, VH, CL, and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature, 341: 544-546; PCT Publication No. WO 90/05144), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, for example, Bird et al. (1988) Science 242: 423-426; and Huston et al. (1988) Proc. Natl. Acad. Sci. USA 85: 5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, for example, Holliger et al. (1993) Proc. Natl. Acad. Sci. USA 90: 6444-6448; Poljak (1994) Structure 2: 1121-1123); Kontermann and Dubel eds., Antibody Engineering, Springer-Verlag, N.Y. (2001), p. 790 (ISBN 3-540-41354-5). In addition, single chain antibodies also include “linear antibodies” comprising a pair of tandem Fv segments (VH-CH1-VH-CH1) which, together with complementary light chain polypeptides, form a pair of antigen binding regions (Zapata et al. (1995) Protein Eng. 8(10): 1057-1062; and U.S. Pat. No. 5,641,870).

An immunoglobulin constant (C) domain refers to a heavy (CH) or light (CL) chain constant domain. Murine and human IgG heavy chain and light chain constant domain amino acid sequences are known in the art.

As used herein, the term “target” refers to a biological molecule (e.g., peptide, polypeptide, protein, lipid, carbohydrate) to which a polypeptide domain which has a binding site can selectively bind. The target can be, for example, an intracellular target (e.g., an intracellular protein target) or a cell surface target (e.g., a membrane protein, a receptor protein). Preferably, a target is a cell surface target, such as a cell surface protein.

As described herein, an “antigen” is a molecule that is bound by a binding site on a polypeptide agent, such as a binding protein, an antibody or antibody fragment, or antigen-binding fragment thereof. Typically, antigens are bound by antibody ligands and are capable of raising an antibody response in vivo. An antigen can be a polypeptide, protein, nucleic acid or other molecule. In the case of conventional antibodies and fragments thereof, the antibody binding site as defined by the variable loops (L1, L2, L3 and H1, H2, H3) is capable of binding to the antigen. The term “antigenic determinant” refers to an epitope on the antigen recognized by an antigen-binding molecule, and more particularly, by the antigen-binding site of said molecule.

The term “epitope” includes any polypeptide determinant capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. An epitope is a region of an antigen that is bound by a binding protein. An epitope may be determined by obtaining an X-ray crystal structure of an antibody:antigen complex and determining which residues on the antigen are within a specified distance of residues on the antibody of interest, wherein the specified distance is, 5 Å or less, e.g., 5 Å, 4 Å, 3 Å, 2 Å, 1 Å or any distance in between. In some embodiments, an “epitope” can be formed on a polypeptide both from contiguous amino acids, or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An “epitope” includes the unit of structure conventionally bound by an immunoglobulin VH/VL pair. Epitopes define the minimum binding site for an antibody, and thus represent the target of specificity of an antibody. In the case of a single domain antibody, an epitope represents the unit of structure bound by a variable domain in isolation. The terms “antigenic determinant” and “epitope” can also be used interchangeably herein. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. In some embodiments, an epitope comprises of 8 or more contiguous or non-contiguous amino acid residues in the sequence in which at least 50%, 70% or 85% of the residues are within the specified distance of the antibody or binding protein in the X-ray crystal structure.

The terms “specificity” or “specific for” refers to the number of different types of antigens or antigenic determinants to which a binding protein, antibody or antibody fragment, or antigen-binding portion thereof thereof as described herein can bind. The specificity of a binding protein, antibody or antibody fragment, or antigen-binding portion thereof thereof can be determined based on affinity and/or avidity. The affinity, represented by the equilibrium constant for the dissociation (KD) of an antigen with an antigen-binding protein, is a measure of the binding strength between an antigenic determinant and an antigen-binding site on the antigen-binding protein, such as a binding protein, antibody or antibody fragment, or antigen-binding portion thereof thereof: the lesser the value of the KD, the stronger the binding strength between an antigenic determinant and the antigen-binding molecule. Alternatively, the affinity can also be expressed as the affinity constant (KA), which is 1/KD). As will be clear to the skilled person, affinity can be determined in a manner known per se, depending on the specific antigen of interest. Accordingly, a binding protein, antibody or antibody fragment, or antigen-binding portion thereof thereof as defined herein is said to be “specific for” a first target or antigen compared to a second target or antigen when it binds to the first antigen with an affinity (as described above, and suitably expressed, for example as a KD value) that is at least 10 times, such as at least 100 times, and preferably at least 1000 times, and up to 10000 times or more better than the affinity with which said amino acid sequence or polypeptide binds to another target or polypeptide.

Accordingly, as used herein, “selectively binds” or “specifically binds” or “specific binding” in reference to the interaction of an antibody, or antibody fragment thereof, or a binding protein described herein, means that the interaction is dependent upon the presence of a particular structure (e.g., an antigenic determinant or epitope or target) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody. In certain embodiments, a binding protein or antibody or antigen-binding fragment thereof that specifically binds to an antigen binds to that antigen with a K_{D} greater than 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, 10⁻¹⁴ M. In other embodiments, a binding protein or antibody or antigen binding fragment thereof that specifically binds to an antigen binds to that antigen with a K_{D} between 10⁻⁶ and 10⁻⁷M, 10⁻⁶ and 10⁻⁸M, 10⁻⁶ and 10⁻⁹ M, 10⁻⁶ and 10⁻¹⁰ M, 10⁻⁶ and 10⁻¹¹ M, 10⁻⁶ and 10⁻¹² M, 10⁻⁶ and 10⁻¹³ M, 10⁻⁶ and 10⁻¹⁴ M, 10⁻⁹ and 10⁻¹⁰ M, 10⁻⁹ and 10⁻¹¹ M, 10⁻⁹ and 10⁻¹² M, 10⁻⁹ and 10⁻¹³ M, 10⁻⁹ and 10⁻¹⁴ M. In some embodiments, a binding protein or antibody or antigen-binding fragment thereof binds to an epitope, with a K_{D} 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the polypeptide agent and the concentration of polypeptide agent. The person of ordinary skill in the art can determine appropriate conditions under which the polypeptide agents described herein selectively bind the targets using any suitable methods, such as titration of a polypeptide agent in a suitable cell binding assay. In certain embodiments, a binding protein or antibody or antigen-binding fragment thereof is said to “specifically bind” an antigen when it preferentially recognizes its target antigen in a complex mixture of proteins and/or macromolecules. Binding proteins, antibodies or antigen-binding fragments that bind to the same or similar epitopes will likely cross-compete (one prevents the binding or modulating effect of the other). Cross-competition, however, can occur even without epitope overlap, e.g., if epitopes are adjacent in three-dimensional space and/or due to steric hindrance.

The term “antibody fragment,” or “antigen-binding fragment” as used herein, refer to a protein fragment that comprises only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., Nature 341, 544-546 (1989)) which consists of a VH domain; (vii) isolated CDR regions; (viii) F(ab′)2 fragments, a bivalent fragment including two Fab′ fragments linked by a disulphide bridge at the hinge region; (ix) single chain antibody molecules (e.g., single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (VH) connected to a light chain variable domain (VL) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (VH-CH1-VH-CH1) which, together with complementary light chain polypeptides, form a pair of antigen binding regions (Zapata et al. Protein Eng. 8(10):1057-1062 (1995); and U.S. Pat. No. 5,641,870).

An “Fv” fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) has the ability to recognize and bind antigen, although usually at a lower affinity than the entire binding site.

The “Fab” fragment contains a variable and constant domain of the light chain and a variable domain and the first constant domain (CH1) of the heavy chain. F(ab′) 2 antibody fragments comprise a pair of Fab fragments which are generally covalently linked near their carboxy termini by hinge cysteines between them. Other chemical couplings of antibody fragments are also known in the art.

“Single-chain Fv” or “scFv” antibody fragments comprise the VH and VL domains of antibody, wherein these domains are present in a single polypeptide chain. Generally the Fv polypeptide further comprises a polypeptide linker between the VH and VL domains, which enables the scFv to form the desired structure for antigen binding. For a review of scFv, see Pluckthun in The Pharmacology of Monoclonal Antibodies, Vol 113, Rosenburg and Moore eds. Springer-Verlag, New York, pp. 269-315 (1994).

The term “monoclonal antibody” or “mAb” as used herein refers to an antibody obtained from a population of substantially homogeneous antibodies, i.e., the individual antibodies comprising the population are identical except for possible naturally occurring mutations that can be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The modifier “monoclonal” is not to be construed as requiring production of the antibody by any particular method. For example, the monoclonal antibodies to be used in accordance with the invention can be made by the hybridoma method first described by Kohler et al., Nature 256:495 (1975), or can be made by recombinant DNA methods (see, e.g., U.S. Pat. No. 4,816,567). The “monoclonal antibodies” can also be isolated from phage antibody libraries using the techniques described in Clackson et al., Nature 352:624-628 (1991) or Marks et al., J. Mol. Biol. 222:581-597 (1991), for example. A monoclonal antibody can be of any species, including, but not limited to, mouse, rat, goat, rabbit, and human monoclonal antibodies. Various methods for making monoclonal antibodies specific for an antigen, such as Notch, as described herein, are available in the art. For example, the monoclonal antibodies can be made using the hybridoma method first described by Kohler et al., Nature, 256:495 (1975), or by recombinant DNA methods (U.S. Pat. No. 4,816,567). “Monoclonal antibodies” can also be isolated from or produced using phage antibody libraries using the techniques originally described in Clackson et al., Nature 352:624-628 (1991), Marks et al., J. Mol. Biol. 222:581-597 (1991), McCafferty et al., Nature, 348:552-554 (1990), Marks et al., Bio/Technology, 10:779-783 (1992)), Waterhouse et al., Nuc. Acids. Res., 21:2265-2266 (1993), and techniques known to those of ordinary skill in the art.

The term “human antibody” includes antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody” does not include antibodies in which CDR sequences derived from the germline of another mammalian species, such as a mouse, have been grafted onto human framework sequences.

The term “recombinant human antibody” includes all human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes, or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “chimeric antibody” refers to antibodies that comprise heavy and light chain variable domain sequences from one species and constant region sequences from another species, such as antibodies having murine heavy and light chain variable domains linked to human constant regions.

The term “CDR-grafted antibody” refers to antibodies that comprise heavy and light chain variable domain sequences from one species but in which the sequences of one or more of the CDR regions of VH and/or VL are replaced with CDR sequences of another species, such as antibodies having murine heavy and light chain variable domains in which one or more of the murine CDRs (e.g., CDR3) has been replaced with human CDR sequences.

The term “CDR” refers to the complementarity determining region within antibody variable sequences. There are three CDRs in each of the variable domains of the heavy chain and the light chain, which are designated CDR1, CDR2 and CDR3, for each of the variable domains. The term “CDR set” as used herein refers to a group of three CDRs that occur in a single variable domain capable of binding the antigen. The exact boundaries of these CDRs have been defined differently according to different systems. The system described by Kabat (Kabat et al., Sequences of Proteins of Immunological Interest, National Institutes of Health, Bethesda, Md. (1987) and (1991)) not only provides an unambiguous residue numbering system applicable to any variable domain of an antibody, but also provides precise residue boundaries defining the three CDRs. These CDRs may be referred to as Kabat CDRs. Chothia and coworkers (Chothia et al. (1987) J. Mol. Biol. 196: 901-917; and Chothia et al. (1989) Nature 342: 877-883) found that certain sub-portions within Kabat CDRs adopt nearly identical peptide backbone conformations, despite having great diversity at the level of amino acid sequence. These sub-portions were designated as L1, L2, and L3 or H1, H2, and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan et al. ((1995) FASEB J. 9:133-139) and MacCallum et al. ((1996) J. Mol. Biol. 262(5):732-745). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although exemplary embodiments use Kabat or Chothia defined CDRs.

As used herein, the term “canonical” residue refers to a residue in a CDR or framework that defines a particular canonical CDR structure as defined by Chothia et al. ((1987) J. Mol. Biol. 196: 901-917); and Chothia et al. ((1992) J. Mol. Biol. 227: 799-817), both are incorporated herein by reference). According to Chothia et al., critical portions of the CDRs of many antibodies have nearly identical peptide backbone confirmations despite great diversity at the level of amino acid sequence. Each canonical structure specifies primarily a set of peptide backbone torsion angles for a contiguous segment of amino acid residues forming a loop.

As used herein, “antibody variable domain” refers to the portions of the light and heavy chains of antibody molecules that include amino acid sequences of Complementarity Determining Regions (CDRs; i.e., CDR1, CDR2, and CDR3), and Framework Regions (FRs). Each heavy chain is composed of a variable region of the heavy chain (VH refers to the variable domain of the heavy chain) and a constant region of said heavy chain. The heavy chain constant region consists of three domains CH1, CH2 and CH3. Each light chain is composed of a variable region of said light chain (VL refers to the variable domain of the light chain) and a constant region of the light chain. The light chain constant region consists of a CL domain. The VH and VL regions can be further divided into hypervariable regions referred to as complementarity-determining regions (CDRs) and interspersed with conserved regions referred to as framework regions (FR). Each VH and VL region thus consists of three CDRs and four FRs that are arranged from the N terminus to the C terminus in the following order: FR1, CDR1, FR2, CDR2, FR3, CDR3, FR4. This structure is well known to those skilled in the art. According to the methods used herein, the amino acid positions assigned to CDRs and FRs can be defined according to Kabat (Sequences of Proteins of Immunological Interest (National Institutes of Health, Bethesda, Md., 1987 and 1991)). Amino acid numbering of antibodies or antigen binding fragments is also according to that of Kabat.

The term “multivalent binding protein” denotes a binding protein comprising two or more antigen binding sites. A multivalent binding protein may be engineered to have three or more antigen binding sites, and is generally not a naturally occurring antibody. The term “multispecific binding protein” refers to a binding protein capable of binding two or more related or unrelated targets.

Similarly, unless indicated otherwise, the expression “multivalent antibody” is used throughout this specification to denote an antibody comprising three or more antigen binding sites. For example, the multivalent antibody is engineered to have the three or more antigen binding sites and is generally not a native sequence IgM or IgA antibody.

In some embodiments, the binding protein is a single chain dual variable domain immunoglobulin protein. The terms “single chain dual variable domain immunoglobulin protein” or “scDVD-Ig protein” or scFvDVD-Ig protein” refer to the antigen binding fragment of a DVD molecule that is analogous to an antibody single chain Fv fragment. scDVD-Ig proteins are described in U.S. Ser. Nos. 61/746,659; 14/141,498 (US application 2014/0243228); and 14/141,500 (US application 2014/0221621), which are incorporated herein by reference in their entireties. In an embodiment, the variable domains of a scDVD-Ig protein are antibody variable domains. In an embodiment, the variable domains are non-immunoglobulin variable domains (e.g., receptor).

In some embodiments, the binding protein is a DVD-Fab. The terms “DVD-Fab” or fDVD-Ig protein” refer to the antigen binding fragment of a DVD-Ig molecule that is analogous to an antibody Fab fragment. fDVD-Ig proteins are described in U.S. Ser. Nos. 61/746,663; 14/141,498 (US Application 2014/0243228); and 14/141,501 (US application US 2014/0235476), incorporated herein by reference in their entireties.

In some embodiments, the binding protein is a receptor DVD-Ig protein. The terms “receptor DVD-Ig protein” constructs, or “rDVD-Ig protein” refer to DVD-Ig constructs comprising at least one receptor-like binding domain. rDVD-Ig proteins are described in U.S. Ser. Nos. 61/746,616; and Ser. No. 14/141,499 (US application 2014/0219913), which are incorporated herein by reference in their entireties.

The term “receptor domain” (RD), or receptor binding domain refers to the portion of a cell surface receptor, cytoplasmic receptor, nuclear receptor, or soluble receptor that functions to bind one or more receptor ligands or signaling molecules (e.g., toxins, hormones, neurotransmitters, cytokines, growth factors, or cell recognition molecules).

The terms multi-specific and multivalent IgG-like molecules or “pDVD-Ig” proteins are capable of binding two or more proteins (e.g., antigens). pDVD-Ig proteins are described in U.S. Ser. No. 14/141,502 (US Application 2014/0213771), incorporated herein by reference in its entirety. In certain embodiments, pDVD-Ig proteins are disclosed which are generated by specifically modifying and adapting several concepts. These concepts include but are not limited to: (1) forming Fc heterodimer using CH3 “knobs-into-holes” design, (2) reducing light chain missing pairing by using CH1/CL cross-over, and (3) pairing two separate half IgG molecules at protein production stage using “reduction then oxidation” approach.

In certain embodiments, a binding protein disclosed herein is a “half-DVD-Ig” comprised of one DVD-Ig heavy chain and one DVD-Ig light chain. The half-DVD-Ig protein preferably does not promote cross-linking observed with naturally occurring antibodies which can result in antigen clustering and undesirable activities. See U.S. Patent Publication No. 2012/0201746 which is incorporated by reference herein in its entirety. In some embodiments, the binding protein is a pDVD-Ig protein. In one embodiment, a pDVD-Ig construct may be created by combining two halves of different DVD-Ig molecules, or a half DVD-Ig protein and half IgG molecule.

In some embodiments, the binding protein is an mDVD-Ig protein. As used herein “monobody DVD-Ig protein” or “mDVD-Ig protein” refers to a class of binding molecules wherein one binding arm has been rendered non-functional. mDVD-Ig proteins are described in U.S. Ser. No. 14/141,503 (US Application 2014/0221622) incorporated herein by reference in its entirety.

The Fc regions of the two polypeptide chains that have a formula of VDH-(X1)n-C-(X2)n may each contain a mutation, wherein the mutations on the two Fc regions enhance heterodimerization of the two polypeptide chains. In one aspect, knobs-into-holes mutations may be introduced into these Fc regions to achieve heterodimerization of the Fc regions. See Atwell et al. (1997) J. Mol. Biol. 270:26-35.

In some embodiments, the binding protein is a cross-over DVD-Ig protein. As used herein “cross-over DVD-Ig” protein or “coDVD-Ig” protein refers to a DVD-Ig protein wherein the cross-over of variable domains is used to resolve the issue of affinity loss in the inner antigen-binding domains of some DVD-Ig molecules. coDVD-Ig proteins are described in U.S. Ser. No. 14/141,504, incorporated herein by reference in its entirety.

The term “bispecific antibody”, as used herein, refers to full-length antibodies that are generated by quadroma technology (see Milstein et al. (1983) Nature 305: 537-540), by chemical conjugation of two different monoclonal antibodies (see Staerz et al. (1985) Nature 314: 628-631), or by knob-into-hole or similar approaches which introduces mutations in the Fc region (see Holliger et al. (1993) Proc. Natl. Acad. Sci. USA 90(14): 6444-6448), resulting in multiple different immunoglobulin species of which only one is the functional bispecific antibody. By molecular function, a bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen binding arms (in both specificity and CDR sequences), and is monovalent for each antigen it binds.

The term “dual-specific antibody”, as used herein, refers to full-length antibodies that can bind two different antigens (or epitopes) in each of its two binding arms (a pair of HC/LC) (see PCT Publication No. WO 02/02773). Accordingly a dual-specific binding protein has two identical antigen binding arms, with identical specificity and identical CDR sequences, and is bivalent for each antigen to which it binds.

A “functional antigen binding site” of a binding protein is one that is capable of binding a target antigen. The antigen binding affinity of the antigen binding site is not necessarily as strong as the parent antibody from which the antigen binding site is derived, but the ability to bind antigen must be measurable using any one of a variety of methods known for evaluating antibody binding to an antigen. Moreover, the antigen binding affinity of each of the antigen binding sites of a multivalent antibody herein need not be quantitatively the same.

As used herein, the terms “donor” and “donor antibody” refer to an antibody providing one or more CDRs. In an exemplary embodiment, the donor antibody is an antibody from a species different from the antibody from which the framework regions are obtained or derived. In the context of a humanized antibody, the term “donor antibody” refers to a non-human antibody providing one or more CDRs.

As used herein, the terms “acceptor” and “acceptor antibody” refer to the antibody providing or nucleic acid sequence encoding at least 80%, at least 85%, at least 90%, at least 95%, at least 98%, or 100% of the amino acid sequences of one or more of the framework regions. In some embodiments, the term “acceptor” refers to the antibody amino acid providing or nucleic acid sequence encoding the constant region(s). In yet another embodiment, the term “acceptor” refers to the antibody amino acid providing or nucleic acid sequence encoding one or more of the framework regions and the constant region(s). In a specific embodiment, the term “acceptor” refers to a human antibody amino acid or nucleic acid sequence that provides or encodes at least 80%, preferably, at least 85%, at least 90%, at least 95%, at least 98%, or 100% of the amino acid sequences of one or more of the framework regions. In accordance with this embodiment, an acceptor may contain at least 1, at least 2, at least 3, least 4, at least 5, or at least 10 amino acid residues that does (do) not occur at one or more specific positions of a human antibody. An acceptor framework region and/or acceptor constant region(s) may be, e.g., derived or obtained from a germline antibody gene, a mature antibody gene, a functional antibody (e.g., antibodies well known in the art, antibodies in development, or antibodies commercially available).

As used herein, the term “germline antibody gene” or “gene fragment” refers to an immunoglobulin sequence encoded by non-lymphoid cells that have not undergone the maturation process that leads to genetic rearrangement and mutation for expression of a particular immunoglobulin. (See, e.g., Shapiro et al. (2002) Crit. Rev. Immunol. 22(3): 183-200; Marchalonis et al. (2001) Adv. Exp. Med. Biol. 484:13-30). One of the advantages provided by various embodiments of the present disclosure stems from the recognition that germline antibody genes are more likely than mature antibody genes to conserve essential amino acid sequence structures characteristic of individuals in the species, hence less likely to be recognized as from a foreign source when used therapeutically in that species.

An “isolated antibody” is intended to refer to an antibody that is substantially free of other antibodies having different antigenic specificities. Moreover, an isolated antibody may be substantially free of other cellular material and/or chemicals.

The term “diabodies” refers to small antibody fragments with two antigen-binding sites, which fragments comprise a heavy chain variable domain (VH) connected to a light chain variable domain (VL) in the same polypeptide chain (VH and VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The term “linear antibodies” refers to the antibodies described in Zapata et al., Protein Eng., 8(10):1057-1062 (1995). Briefly, these antibodies comprise a pair of tandem Fd segments (VH-CH1-VH-CH1) which, together with complementary light chain polypeptides, form a pair of antigen binding regions. Linear antibodies can be bispecific or monospecific.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

The term “humanized antibody” refers to antibodies that comprise heavy and light chain variable domain sequences from a non-human species (e.g., a mouse) but in which at least a portion of the VH and/or VL sequence has been altered to be more “human-like”, i.e., more similar to human germline variable sequences. Accordingly, “humanized” antibodies are a form of a chimeric antibody, that are engineered or designed to comprise minimal sequence derived from non-human immunoglobulin. For the most part, humanized antibodies are human immunoglobulins (recipient or acceptor antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies can comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). As used herein, a “composite human antibody” or “deimmunized antibody” are specific types of engineered or humanized antibodies designed to reduce or eliminate T cell epitopes from the variable domains.

One type of humanized antibody is a CDR-grafted antibody, in which human CDR sequences are introduced into non-human VH and VL sequences to replace the corresponding nonhuman CDR sequences. Also “humanized antibody” is an antibody or a variant, derivative, analog or fragment thereof which immunospecifically binds to an antigen of interest and which comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having an amino acid sequence at least 80%, at least 85%, at least 90%, at least 95%, at least 98% or at least 99% identical to the amino acid sequence of a non-human antibody CDR. A humanized antibody comprises substantially all of at least one, and typically two, variable domains (Fab, Fab′, F(ab′).sub.2, FabC, Fv) in which all or substantially all of the CDR regions correspond to those of a non-human immunoglobulin (i.e., donor antibody) and all or substantially all of the framework regions are those of a human immunoglobulin consensus sequence. In an embodiment, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain. A humanized antibody may be selected from any class of immunoglobulins, including IgM, IgG, IgD, IgA and IgE, and any isotype including without limitation IgG1, IgG2, IgG3, and IgG4. The humanized antibody may comprise sequences from more than one class or isotype, and particular constant domains may be selected to optimize desired effector functions using techniques well known in the art.

With respect to constructing DVD-Ig or other binding protein molecules, a “linker” is used to denote a single amino acid or a polypeptide (“linker polypeptide”) comprising two or more amino acid residues joined by peptide bonds and used to link one or more antigen binding portions. Such linker polypeptides are well known in the art (see, e.g., Holliger et al. (1993) Proc. Natl. Acad. Sci. USA 90: 6444-6448; Poljak (1994) Structure 2: 1121-1123).

A “human antibody,” “non-engineered human antibody,” or “fully human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art. In one embodiment, the human antibody is selected from a phage library, where that phage library expresses human antibodies (Vaughan et al. Nature Biotechnology 14:309-314 (1996): Sheets et al. Proc. Natl. Acad. Sci. 95:6157-6162 (1998)); Hoogenboom and Winter, J. Mol. Biol., 227:381 (1991); Marks et al., J. Mol. Biol., 222:581 (1991)). Human antibodies can also be made by introducing human immunoglobulin loci into transgenic animals, e.g., mice in which the endogenous mouse immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368:812-13 (1994); Fishwild et al., Nature Biotechnology 14: 845-51 (1996); Neuberger, Nature Biotechnology 14: 826 (1996); Lonberg and Huszar, Intern. Rev. Immunol. 13:65-93 (1995). Alternatively, the human antibody can be prepared via immortalization of human B lymphocytes producing an antibody directed against a target antigen (such B lymphocytes can be recovered from an individual or can have been immunized in vitro). See, e.g., Cole et al., Monoclonal Antibodies and Cancer Therapy, Alan R. Liss, p. 77 (1985); Boerner et al., J. Immunol., 147 (1):86-95 (1991); and U.S. Pat. No. 5,750,373.

An “affinity matured” antibody is one with one or more alterations in one or more CDRs thereof which result an improvement in the affinity of the antibody for antigen, compared to a parent antibody which does not possess those alteration(s). Exemplary affinity matured antibodies will have nanomolar or even picomolar affinities for the target antigen. A variety of proceedures for producing affinity matured antibodies are known in the art. For example, Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al., J. Mol. Biol. 226:889-896 (1992). Selective mutation at selective mutagenesis positions and at contact or hypermutation positions with an activity enhancing amino acid residue is described in U.S. Pat. No. 6,914,128.

A “functional antigen binding site” of an antibody is one which is capable of binding a target antigen. The antigen binding affinity of the antigen binding site is not necessarily as strong as the parent antibody from which the antigen binding site is derived, but the ability to bind antigen must be measurable using any one of a variety of methods known for evaluating antibody binding to an antigen. Moreover, the antigen binding affinity of each of the antigen binding sites of a multivalent antibody herein need not be quantitatively the same. For multimeric antibodies, the number of functional antigen binding sites can be evaluated using ultracentrifugation analysis as described in Example 2 of U.S. Patent Application Publication No. 20050186208. According to this method of analysis, different ratios of target antigen to multimeric antibody are combined and the average molecular weight of the complexes is calculated assuming differing numbers of functional binding sites. These theoretical values are compared to the actual experimental values obtained in order to evaluate the number of functional binding sites.

As used herein, a “blocking” or “neutralizing” binding protein, antibody, antibody fragment, antigen-binding fragment or an antibody “antagonist” is one which inhibits or reduces the biological activity of the antigen it specifically binds to the antigen. In certain embodiments, blocking or neutralizing antibodies or antagonist antibodies completely inhibit the biological activity of the antigen. The neutralizing binding protein, antibody, antigen-binding fragment thereof can bind a target, such as Notch, and reduce a biological activity by at least about 20%, 40%, 60%, 80%, 85%, or more. Inhibition of a Notch biological activity by a neutralizing binding protein, antibody or antigen-binding fragment thereof can be assessed by measuring one or more indicators of Notch biological activity well known in the art.

An antibody having a “biological characteristic” or “functional characteristic” of a designated antibody is one which possesses one or more of the biological properties of that antibody which distinguish it from other antibodies that bind to the same antigen, including, for example, binding to a particular epitope, an EC50 value, IC50 value or KD values, as defined elsewhere herein.

In order to screen for antibodies which bind to an epitope on an antigen bound by an antibody of interest, a routine cross-blocking assay such as that described in Antibodies, A Laboratory Manual, Cold Spring Harbor Laboratory, Ed Harlow and David Lane (1988), can be performed.

As used herein, “antibody mutant” or “antibody variant” refers to an amino acid sequence variant of the species-dependent antibody wherein one or more of the amino acid residues of the species-dependent antibody have been modified. Such mutants necessarily have less than 100% sequence identity or similarity with the species-dependent antibody. In one embodiment, the antibody mutant will have an amino acid sequence having at least 75% amino acid sequence identity or similarity with the amino acid sequence of either the heavy or light chain variable domain of the species-dependent antibody, more preferably at least 80%, more preferably at least 85%, more preferably at least 90%, and most preferably at least 95%. Identity or similarity with respect to this sequence is defined herein as the percentage of amino acid residues in the candidate sequence that are identical (i.e., same residue) or similar (i.e., amino acid residue from the same group based on common side-chain properties, see below) with the species-dependent antibody residues, after aligning the sequences and introducing gaps, if necessary, to achieve the maximum percent sequence identity. None of N-terminal, C-terminal, or internal extensions, deletions, or insertions into the antibody sequence outside of the variable domain shall be construed as affecting sequence identity or similarity.

As used herein, a “targeting sequence” refers to a polypeptide sequence sufficient to direct the localization of, e.g., a polypetide, to a specific subcellular localization. By way of example, a “targeting sequence” can direct the polypeptide to the, e.g., a transmembrane domain, or to the nucleus, e.g., a nuclear localization sequence. A targeting sequence can be added to a biological molecule (e.g., peptide, polypeptide, protein, lipid, carbohydrate) to direct the polypeptides localization. A targeting sequence can result in the irreversible or reversible localization of a polypeptide.

An “isolated” antibody is one that has been identified and separated and/or recovered from a component of its natural environment. Contaminant components of its natural environment are materials that would interfere with diagnostic or therapeutic uses for the antibody, and can include enzymes, hormones, and other proteinaceous or non-proteinaceous solutes. In certain embodiments, the antibody will be purified (1) to greater than 95% by weight of antibody as determined by, for example, the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, silver stain. Isolated antibody includes the antibody in situ within recombinant cells since at least one component of the antibody's natural environment will not be present. Ordinarily, however, isolated antibody will be prepared by at least one purification step.

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). See also Jonsson U. et al., (1993) Ann. Biol. Clin., 51:19-26; Jonsson U. et al., (1991) BioTechniques, 11:620-627 (1991); Johnsson U. et al., (1995) J. Mol. Recognit., 8:125-131; and Johnsson U. et al., (1991) Anal. Biochem., 198:268-277.

The term “binding protein conjugate” or “antibody conjugate” refers to a binding protein or antibody or antigen-binding fragment thereof as described herein chemically linked to a second chemical moiety, such as a therapeutic or cytotoxic agent. The term “agent” is used herein to denote a chemical compound, a mixture of chemical compounds, a biological macromolecule, or an extract made from biological materials. Preferably the therapeutic or cytotoxic agents include, but are not limited to, anti-cancer therapies as discussed herein, as well as pertussis toxin, taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. When employed in the context of an immunoassay, a binding protein conjugate or antibody conjugate may be a detectably labeled antibody, which is used as the detection antibody.

The term “cytotoxic agent” as used herein refers to a substance that inhibits or prevents the function of cells and/or causes destruction of cells. The term is intended to include radioactive isotopes (e.g. At211, I131, I125, Y90, Re186, Re188, Sm153, Bi212, P32 and radioactive isotopes of Lu), chemotherapeutic agents, and toxins such as small molecule toxins or enzymatically active toxins of bacterial, fungal, plant or animal origin, including fragments and/or variants thereof.

The terms “crystal” and “crystallized” as used herein, refer to a binding protein, antibody or antigen-binding protein, or antigen binding portion thereof, that exists in the form of a crystal. Crystals are one form of the solid state of matter that is distinct from other forms such as the amorphous solid state or the liquid crystalline state. Crystals are composed of regular, repeating, three-dimensional arrays of atoms, ions, molecules (e.g., proteins such as DVD-Igs), or molecular assemblies (e.g., antigen/binding protein complexes).

By “fragment” is meant a portion of a polypeptide, such as a binding protein, antibody or antibody fragment, or antigen-binding portion thereof thereof, or nucleic acid molecule that contains, preferably, at least 10%, 20%, 30%, 40%, 50%, 60%, 70%, 80%, 90%, 95%, or more of the entire length of the reference nucleic acid molecule or polypeptide. A fragment can contain 10, 20, 30, 40, 50, 60, 70, 80, 90, or 100, 200, 300, 400, 500, 600, or more nucleotides or 10, 20, 30, 40, 50, 60, 70, 80, 90, 100, 120, 140, 160, 180, 190, 200 amino acids or more.

By “reduce” or “inhibit” is meant the ability to cause an overall decrease preferably of 10% or greater, 15% or greater 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 98% or greater, 99% or greater, or complete or 100% in a parameter, activity, or condition being measured.

The terms “cell lines,” “host cells,” and “host cells lines” refer to cells that can be genetically engineered to express a nucleic acid sequence encoding any of the synthetic proteins or components thereof described herein. Cell lines are typically derived from a lineage arising from a primary culture that can be maintained in culture for an unlimited time. Genetically engineering the cell line involves transfecting, transforming or transducing the cells with a recombinant polynucleotide molecule, and/or otherwise altering (e.g., by homologous recombination and gene activation or fusion of a recombinant cell with a non-recombinant cell) so as to cause the host cell to express a synthetic protein of interest.

The term “mammalian host cell” is used to refer to a mammalian cell which is capable of being transfected with a nucleic acid sequence and then of expressing a selected recombinant protein of interest. The term includes the progeny of the parent cell, whether or not the progeny is identical in morphology or in genetic make-up to the original parent, so long as the selected gene is present. Suitable mammalian cells for use in the present invention include, but are not limited to Chinese hamster ovary (CHO) cells, baby hamster kidney (BHK) cells, human HeLa cells, monkey COS-1 cell, human embryonic kidney 293 cells, mouse myeloma NSO and human HKB cells (U.S. Pat. No. 6,136,599). The other cell lines are readily available from the ATCC.

The term “recombinant” when used with reference to a cell indicates that the cell replicates a heterologous nucleic acid, or expresses a peptide or protein encoded by a heterologous nucleic acid, such as a nucleic acid sequence encoding a synthetic Notch protein, i.e., a nucleic acid sequence encoding any such recombinant protein of interest. Recombinant cells can contain genes that are not found within the native (non-recombinant) form of the cell. Recombinant cells can also contain genes found in the native form of the cell wherein the genes are modified and reintroduced into the cell by artificial means. The term also encompasses cells that contain a nucleic acid endogenous to the cell that has been modified without removing the nucleic acid from the cell; such modifications include those obtained by gene replacement, site-specific mutation, using techniques such as Crispr, and related techniques. A “recombinant protein” is one which has been produced by a recombinant cell.

As used herein, the terms “recombinant cell,” “recombinant cell line,” or “modified cell line” refers to a cell line either transiently or stably transformed with one or more nucleic acid constructs, as described herein. Polynucleotides, genetic material, recombinant DNA molecules, expression vectors, and such, used in the compositions and methods described herein can be isolated using standard cloning methods such as those described by Sambrook et al. (Molecular Cloning: A Laboratory Manual, Second Edition, Cold Spring Harbor, N.Y., 1989; which is incorporated herein by reference). Alternatively, the polynucleotides coding for a recombinant protein product used in the compositions and methods described herein can be synthesized using standard techniques that are well known in the art, such as by synthesis on an automated DNA synthesizer.

Peptides, polypeptides and proteins that are produced by recombinant animal cell lines using the cell culture compositions and methods described herein can be referred to as “recombinant protein of interest,” “recombinant peptide,” “recombinant polypeptide,” and “recombinant protein.” The expressed protein(s) can be produced intracellularly or secreted into the culture medium from which it can be recovered and/or collected. Accordingly, the term “recombinant protein of interest” refers to a protein or fragment thereof expressed from an exogenous nucleic acid sequence introduced into a host cell.

As used herein, the term “transfection” is used to refer to the uptake of an exogenous nucleic acid by a cell, and a cell has been “transfected” when the exogenous nucleic acid has been introduced inside the cell membrane. A number of transfection techniques are well known in the art and are disclosed herein.

The term “transformation” as used herein refers to a change in a cell's genetic characteristics, and a cell has been transformed when it has been modified to contain a new DNA. For example, a cell is transformed where it is genetically modified from its native state. Following transfection, the transforming nucleic acid can recombine with that of the cell by physically integrating into a chromosome of the cell, can be maintained transiently as an episomal element without being replicated, or can replicate independently as a plasmid. A cell is considered to have been stably transformed when the transforming nucleic acid is replicated with the division of the cell.

As used herein an “expression vector” refers to a DNA molecule, or a clone of such a molecule, which has been modified through human intervention to contain segments of DNA combined and juxtaposed in a manner that would not otherwise exist in nature. In addition, an expression vector can comprise additional DNA segments, such as promoters, transcription terminators, enhancers, and other elements. One or more selectable markers can also be included. DNA constructs useful for expressing cloned DNA segments in a variety of prokaryotic and eukaryotic host cells can be prepared from readily available components or purchased from commercial suppliers.

By “cell culture” or “culture” is meant the growth and propagation of cells outside of a multicellular organism or tissue. Suitable culture conditions for mammalian cells are known in the art. See e.g. Animal cell culture: A Practical Approach, D. Rickwood, ed., Oxford University Press, New York (1992). Mammalian cells can be cultured in suspension or while attached to a solid substrate. Fluidized bed bioreactors, hollow fiber bioreactors, roller bottles, shake flasks, or stirred tank bioreactors, with or without microcarriers, can be used.

As used herein, “cell culture medium” is a media suitable for growth of animal cells, such as mammalian cells, in in vitro cell culture. Cell culture media formulations are well known in the art. Typically, cell culture media are comprised of buffers, salts, carbohydrates, amino acids, vitamins and trace essential elements. “Serum-free” applies to a cell culture medium that does not contain animal sera, such as fetal bovine serum. Various tissue culture media, including defined culture media, are commercially available.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 presents a structure schematic of the Notch Regulatory Region (NRR). The NRR structure with rainbow spectrum coloring. Subregions of the structure are listed: LNR-A, LNR-B, LNR-C, heterodimerization domain (HD). The Ala-Val side-chain residues containing the S2 scissile bond are shown in magenta. Spheres are the Ca⁺² ions coordinated by the LNR domains. Structure is of mouse Notch1 NRR. PBD: 7ABV.

FIGS. 2A-2C present information regarding receptors containing protease linker insertions within the NRR are either poorly surface expression or exhibit high basal activity. U2OS reporter cell line (UAS:dsRed2) were transduced with viral particles encoding a HA-Halo-NRR-GAL4VP64 receptor with a TEVs substrate ‘LEGGGSENLYFQSGRTGGGS’ (SEQ ID NO: 38) inserted in indicated positions within the NRR, co-expressing tagBFP with a ‘T2A’ self-cleaving peptide. Viral particles encoding ‘T2A-tagBFP’ sequence was used as a no-receptor control. SynNotch denotes control receptor with no TEVs insertion. TEVs linker was inserted between (1) LNR-B and LNR-C (2) LNR-C and HD-N (3) between the cysteine ‘knuckle’ and α3 helix of HD-C and (4) between α3 and β5 of HD-C. (FIG. 2A) Arrows point to the TEVs insertion sites within the NRR structure. PDB:7ABV Cells were analyzed by flow for (FIG. 2B) surface expression (Halo-JF635i) and (FIG. 2C) dsRed2 reporter activities 48 days post transduction. Traces represent normalized densities of BFP+ population with median value shown (black solid lines). Thresholds for BFP and JF635i calculated from non-transduced cells (dashed lines).

FIG. 3 presents a schematic of proteolytic regulation of proteins. Schematic illustrating the principle of protease-driven maturation and activation of proteins in natural systems. Concept is extended to protease-activated small molecule drugs and biologics. Figure adapted from Bleuez et al. 2022, which is incorporated herein by its entirety.

FIG. 4 presents a schematic of the structural model of anti-NRR1 bound to NOTCH1 NRR.

FIG. 5 presents a schematic of the proposed design of a protease activated receptor inspired by the Notch activation mechanism. An inhibitory mask (*) fused to a hyperactive notch variant (arrow) with a cleavable linker (\hat{ }) is expected to remain quiescent at the cell-surface. In the presence of an extracellular protease (***), the cleavable linker is expected to be hydrolyzed and induce dissociation of the inhibitory mask. Once the mask dissociates, the hyperactive Notch is cleaved by ADAM at S2 without requiring force, since NRR is not autoinhibited. S3 cleavage occurs resulting in ICD release from the membrane and subsequence gene regulation.

FIGS. 6A and 6B present schematics of protease-activation screen in U2OS reporter cell line using TEV protease. (FIG. 6A) Schematic showing receptor design—an N-terminal mask domain based on the anti-NRR1 scFv previously used in sNRR and TEV substrate (TEVs) ‘ENLYFQS’ (SEQ ID NO: 39) fused to an NRR with a destabilizing T-ALL mutation, and GAL4 based transcription factor ICD. U2OS with an integrated UAS:dsRed2 reporter gene will be transduced with pHR lentiviral encoding receptors to evaluate the designs. (FIG. 6B) Structure of the mouse NRR1: LNRs, coordinated Ca⁺² ions, HD-N, HD-C and the destabilizing mutations found in human NOTCH1 T-ALL cancers. Secondary structural elements with T-ALL mutations tested in our receptor designs are labelled using the same designations as Gordon et al 2007²⁰ PBD:7ABV

FIGS. 7A and 7B present data showing receptor design activation and surface expression. U2OS reporter cell line (UAS:dsRed2) were transduced with viral particles encoding receptors with TEVs substrate co-expressing BFP with a ‘T2A’ self-cleaving peptide. ‘-scFv control’ denotes no masking domain control (ie, HA-Halo-NRR-GAL4VP64). The ‘A2’, ‘A3’, and ‘A’ denote the anti-NRR1 scFv variant with varying affinities against NRR reported in Table 1. ‘BFP’ denotes cells transduced with a ‘T2A-BFP’ construct. The ‘WT’ denotes the non-mutated NRR control receptor (ie., HA-Halo-antiNRR-TEVs-NRR-GAL4VP64). NRR mutation denoted otherwise. 48 hours after transduction, media was replaced with optiMEM or optiMEM+TEVp (NEB, 1:100 dilution). 24 hours after media exchange, cells were analyzed by flow cytometry. Traces represent normalized densities of BFP+ population (>5,000 cells analyzed). (FIG. 7A) Reporter activation and (FIG. 7B) surface expression shown with median value (solid black line). Thresholds for dsRed2 and JF635i fluorescence values calculated from non-transduced control cells shown (dashed line).

FIG. 8 presents data showing ‘A-2’ anti-NRR1 scFv based receptor designs TEVp-dependent activation. Subset of data presented in FIG. 7 showing TEVp-mediated activation of the receptor designs using the ‘A-2’ variant of the anti-NRR1 scFv with the various T-ALL destabilizing NRR mutations. The NRR variants are shown in order of in vitro NRR dissociation reported by Malecki et al. 2006. ‘no receptor’ denotes cells transduced with viral particles encoding ‘T2A-BFP’.

FIG. 9 presents data showing ‘A-2’ anti-NRR1 scFv based receptor designs are activated by plated ligand. U2OS reporter cell line (UAS:dsRed2) were transduced with viral particles encoding receptors with TEVs substrate co-expressing BFP with a ‘T2A’ self-cleaving peptide. ‘WT’ denotes the non-mutated NRR receptor control. 48 hours after transduction, cells were replated on indicated plated ligand condition using versene passaging non-enzymatic reagent. ‘biotin-halo’ denotes plate-bound biotin-halo-ligand via plate-adsorbed neutravidin. Halo-ligand is expected to covalently react to HaloTag in receptor ECDs and result in plated ligand-mediated signaling. Cells were analyzed by flow cytometry 48 hours after ligand stimulation. Traces represent normalized densities of BFP+ population (>5,000 cells analyzed). Reporter activation shown with median value (solid black line). Thresholds for dsRed2 fluorescence calculated from non-transduced control cells shown (dashed line). FN; fibronectin control.

FIG. 10 presents data showing ‘A-2’ anti-NRR1 scFv based receptor designs are all processed by furin. HEK293-FT cells were transiently transfected with pcDNA3 based plasmids encoding the indicated receptors and immunoblotted with an anti-GAL4 antibody. syN: SynNotch, WT: non-mutated NRR control receptor fused to the anti-NRR1 scFv. L1575P, L1594P, L1601P, V1677D, L1679P, 11681N, and A1702P indicate those particular NRR mutants fused to the anti-NRR1 scFv.

FIGS. 11A and 11B present data showing ‘A-2’ anti-NRR1 scFv dissociates upon cleavage. HEK293-FT cells were transfected with pcDNA3-based plasmids encoding the indicated receptor with TEVs substrate linker and N-terminal HA tag. (FIG. 11A) cell lysates and conditioned anti-HA immunoblots after 3 hours of TEVp incubation, overwise indicated. FL; full-length receptor, NTF; N-terminal fragment, Ab; ‘A-2’ variant of the anti-NRR1 scFv. (FIG. 11B) Inventors further evaluated the efficiency of TEVs cleavage by comparing an anti-HA blot (which stains all copies of the receptor, including those in the secretory pathway) to a streptavidin blot probing for surface-bound receptor copies by labelling receptors with a cell-impermeant halo-ligand-biotin conjugate. This showed all the NTF surface copies are efficiently cleaved. Immunoblot of cell lysates and conditioned media of HEK293-FT expressing the ‘V1677D’ design by transient transfection. Cells were stained with cell-impermeant halo-biotin prior to TEVp addition. Cell lysates and conditioned media were collected for immunoblot analysis after 3 hours. Colored triangle markers denote FL (top arrow of top blot), NTF (middle arrow of top blot; top arrow of bottom blot) and cleaved NTF (bottom arrows of both blots) species.

FIG. 12 presents data showing that P12 and JME17 NRR insertions are not inhibited by the ‘A-2’ anti-NRR1 scFv mask domain. U2OS reporter cell line (UAS:dsRed2) were transduced with viral particles encoding receptors with TEVs substrate co-expressing BFP with a ‘T2A’ self-cleaving peptide as shown, and analyzed similar to FIG. 7. ‘P12’ is an in frame duplication of the ADAM10 S2 cleavage site within the NRR. ‘JME17’ is an in frame extension of the Notch extracellular juxtamembrane region. Unlike the other T-ALL mutation shown in FIG. 37, they are not expected to be inhibited by the anti-NRR1 scFv mask. ‘WT’ and ‘V1677D’ shown replicated from FIG. 7 for comparison.

FIGS. 13A-13C present data showing TEVs-ProNotch quantified activation. U2OS reporter cell line (UAS:dsRed2) were transduced with viral particles encoding receptors with TEVs substrate ProNotch (TEVs-ProNotch) co-expressing BFP with a ‘T2A’ self-cleaving peptide. Media was replaced 48 post-transduction and replaced with optiMEM+/−TEVp (NEB, 1:100). Cells were analyzed for dsRed2 expression 24 hours later. (FIG. 13A) Traces represent normalized densities of BFP+ population of 3 replicate transductions (>5,000 cells analyzed per replicate). (FIG. 13B) Median reporter intensities (dsRed2) and (FIG. 13C) percent dsRed+ from (FIG. 13A). Displayed values were analyzed using t-test between the two conditions. **** indicated p<0.0001.

FIG. 14 presents data showing TEVs-proNotch activation by vendor TEVp (NEB) compared to recombinantly expressed and purified TEVp. U2OS reporter cell line (UAS:dsRed2) were transduced with viral particles encoding TEVs-ProNotch with ‘T2A-BFP’ co-expression marker. 48 hours after transduction media was replaced with indicated condition. ‘control’ indicated optiMEM control. Cells were analyzed for dsRed2 expression 24 hours later. Traces represent normalized densities of BFP+ population (>5,000 cells per condition). Median dsRed2 values of BFP+ population indicated by black vertical line. Thresholds for BFP and dsRed2 (dashed line) calculated from non-transduced control cells.

FIG. 15 presents data showing human NOTCH1 NRR based ProNotch activation by TEVp. U2OS reporter cell line (UAS:dsRed2) were transduced with viral particles encoding receptors with TEVs substrate ProNotch (TEVs-ProNotch) with either human or mouse NRR-V1677D co-expressing BFP with a ‘T2A’ self-cleaving peptide. Media was replaced 48 post-transduction and replaced with optiMEM+/−TEVp (NEB, 1:100). Cells were analyzed for dsRed2 expression 24 hours later. Traces represent normalized densities of BFP+ population (>5,000 cells analyzed). Thresholds for BFP and dsRed2 (dashed line) calculated from non-transduced control cells.

FIG. 16 presents data showing Notch processing inhibition decreases TEVp-mediated signaling. U2OS reporter cell line (UAS:dsRed2) were transduced with viral particles encoding TEVs-ProNotch with ‘T2A-BFP’ co-expression marker. 48 hours after transduction media was replaced with indicated condition. ‘control’ indicated optiMEM control without TEVp (NEB, 1:100). Cells were analyzed for dsRed2 expression 24 hours later. Traces represent normalized densities of BFP+ population (>5,000 cells per condition). Median dsRed2 values of BFP+ population indicated by black vertical line. Thresholds for BFP and dsRed2 (dashed line) calculated from non-transduced control cells.

FIG. 17 presents data showing that the anti-NRR1 scFv mask keeps destabilizing NRR autoinhibited. U2OS reporter cell line (UAS:dsRed2) were transduced with viral particles encoding indicated receptor with ‘T2A-BFP’ co-expression marker. 48 hours after transduction media was replaced with indicated condition. ‘control’ indicated optiMEM control without TEVp (NEB, 1:100). Cells were analyzed for dsRed2 expression 24 hours later. Traces represent normalized densities of BFP+ population (>5,000 cells per condition). Median dsRed2 values of BFP+ population indicated by black vertical line. Thresholds for BFP and dsRed2 (dashed line) calculated from non-transduced control cells. EDTA; 0.5 mM

FIG. 18 presents data showing non-enzymatic dissociative agents required to prevent spontaneous activation of ProNotch receptor expressing cells. U2OS reporter cell line (UAS:dsRed2) were transduced with viral particles encoding HA-Halo-SynNotch, TEVs-ProNotch, and non-mutated NRR control (sNRR) with ‘T2A-BFP’ co-expression marker. 48 hours later one cell population was passaged with the indicated dissociative agent, and a control population was either rinsed with media or trypsin for 5 minutes. Levels of ligand-independent activation of dsRed2 reporter show. Inset shows BFP intensities. Scale bar=200 μm

FIG. 19 presents data showing TEVp-mediated activation in U2OS and HEK293-FT stably expressing TEVs-ProNotch populations. A U2OS reporter clone (UAS:dsRed2) and HEK293-FT reporter clone (UAS:H2B-mCherry) were transduced with TEVs-ProNotch with an EF1α promoter with a co-expressing BFP via a ‘T2A’ self-cleaving peptide. Receptor expressing pools were derived after hygromycin selection. Cells were plated using a non-enzymatic dissociative agent (versene) and media was exchanged the following day with or without TEVp (1:100 dilution in optiMEM). Images were taken 24 hours after media exchange. Scale bar=200 μm

FIG. 20 presents schematic showing soluble TEVp induced myogenic conversion of C3H10T1/2 fibroblasts via ProNotch signaling. Schematic depicting TEVs-ProNotch containing a TetR-VP48 ICD. The receptor is expressed in C3H10T1/2 fibroblasts containing a TRE-regulated target gene encoding p65-MyoD-T2A-DsRed2. Depiction of the signaling-mediated conversion of C3H10T1/2 fibroblasts in response to TEVp-induced TEVs-ProNotch activation. Expression of p65-MyoD-T2A-DsRed2 results in the formation of red fluorescent syncytia exhibiting myogenic phenotypes.

FIG. 21 presents data showing conditional myogenic conversion by soluble TEVp-mediated TEVs-ProNotch-tTA signaling. C3H10T1/2 fibroblasts were transduced with lentiviral particles encoding TEVs-proNotch-tTA-T2A-BFP. After 48 hours post transfection, media was replaced with indicated condition, optiMEM+/−TEVp (NEB, 1:100). Cells were imaged 24 hours after media exchange. DsRed2 intensities shown in magenta, inset shows receptor BFP co-expression marker in grayscale. Similar treatment of C3H10T1/2 cells containing a TRE:DsRed2 reporter gene (in place of TRE:p65-MyoD-T2A-DsRed2) resulted in confined DsRed2 expression without discernible syncytia formation. Scale bar=200 μm.

FIG. 22 presents schematic showing proNotch receptor can be specified for different proteases by exchanging protease substrate. Schematic depicted a ProNotch receptor with a user-defined protease substrate linker shown in orange.

FIGS. 23A-23C present data showing soluble Factor Xa protease activates the FactorXa-ProNotch receptor. U2OS reporter cell line (UAS:dsRed2) were transduced with viral particles encoding receptors with Factor Xa substrate ProNotch (Factor Xa-ProNotch) co-expressing BFP with a ‘T2A’ self-cleaving peptide. Media was replaced 48 post-transduction and replaced with media+/− Factor Xa at the indicated concentration (NEB). Cells were analyzed for dsRed2 expression 24 hours later. (FIG. 23A) Traces represent normalized densities of BFP+ population of 3 replicate transductions (>5,000 cells analyzed per replicate) with median dsRed intensity shown (black vertical line). (FIG. 23B) Median reporter intensities (dsRed2) and (FIG. 23C) percent dsRed+ from (FIG. 23A). Displayed values were analyzed using one-way ANOVA. p<0.0001 for all unlabeled comparisons. labelled n.s.; p>0.05, **; p<0.005, otherwise.

FIG. 24 presents data showing that a protease substrate can be substituted to specify ProNotch activating protease. U2OS reporter cell line (UAS:dsRed2) were transduced with viral particles encoding indicated receptor with ‘T2A-BFP’ co-expression marker. 48 hours after transduction media was replaced with indicated condition. ‘ProNotch-control’ indicates receptor with myc-tag linker. ‘-’ denotes optiMEM control without any protease. Cells were analyzed for dsRed2 expression 24 hours later. Traces represent normalized densities of BFP+ population (>5,000 cells per condition). Median dsRed2 values of BFP+ population indicated by black vertical line. Thresholds for BFP and dsRed2 (dashed line) calculated from non-transduced control cells. TEVp; 1:100, enterokinase; 10 μg/mL, Factor Xa; 10 μg/mL.

FIG. 25 presents data showing U2OS endogenous activation of MMP2/9-ProNotch. U2OS reporter cell line (UAS:dsRed2) were transduced with viral particles encoding indicated receptor with ‘T2A-BFP’ co-expression marker. 24 hours after transduction media was replaced with indicated condition. Cells were analyzed for dsRed2 expression 24 hours after media exchange. Traces represent normalized densities of BFP+ population (>5,000 cells per condition). Median dsRed2 values of BFP+ population indicated by black vertical line. Thresholds for BFP and dsRed2 (dashed line) calculated from non-transduced control cells.

FIGS. 26A and 26B present data showing MMP-2 specifically activates HEK-MMP2/9-ProNotch. (FIG. 26A) Clonal HEK293-FT MMP2/9-ProNotch and scramble control-ProNotch cell lines with ‘T2A-BFP’ co-expression marker are surface expressed. Live cells were stained with Halo-JF635i. Inset shows overlay of BFP and brightfield intensities. Scale bar=200 μm. (FIG. 26B) Cells were plated, and after 24 hours media was replaced with indicated concentration of pre-activated MMP-2. Cells were analyzed by flow after overnight incubation. Traces represent normalized densities of BFP+ population of 3 replicate MMP-2 treatments (>5,000 cells analyzed per replicate). Median mCherry intensities shown (vertical line). Thresholds for BFP and mCherry (dashed line) calculated from non-transduced control cells.

FIGS. 27A-27F present data showing HEK-MMP2/9-ProNotch activated in coculture by HT1080 cells expressing gelatinases. (FIG. 27A) The clonal HEK293-FT (UAS:H2B-mCherry) MMP2/9-ProNotch and scramble control-ProNotch cell lines were treated with or without 100 nM PMA. (FIG. 27B) The ProNotch cells were co-plated in a 2:1 ratio with HT1080 cells in a 96-well (30,000 HEK receiver and 15,000 HT1080). The next morning, media was exchanged with the indicated condition, and cells were analyzed 48 hours later by flow cytometry. Traces represent normalized densities of the mCherry intensities of the BFP+ population, with median value shown (vertical black line) of three independent drug treatments/cocultures (n=3, >5,000 analyzed per replicate). The percent mCherry+ of BFP+ population is shown in red. The (FIG. 27C) percent mCherry+ and (FIG. 27D) median mCherry intensities of the HEK-MMP2/9-ProNotch cells (BFP+) in coculture with HT1080 as done in (FIG. 27B) with 0 or 6.25 nM PMA. t-test was performed showing statistical signaling between conditions. **; p<0.001, ****; p<0.0001 (FIG. 27E) Comparing the signaling of HEK-MMP2/9-ProNotch cells cocultured with HT1080+100 nM PMA with various MMP inhibitors. Mean percent mCherry+ of receiver population shown of 3 independent coculture drug treatments. Displayed values were analyzed using one-way ANOVA. All unlabeled comparisons have p<0.0001. n.s. p>0.05. GM6001; 125 nM, PD-166793; 1 μM, TIMP2; 5 μg/mL, TIMP1; μg/mL. (FIG. 27F) HEK293-MMP2/9-ProNotch cells were treated with the indicated condition. Mean percent mCherry+ of receptor cells (BFP+) shown of 3 independent protease treatments. Displayed values were analyzed using one-way ANOVA. Labelled n.s.; p>0.05, ***; p<0.001,****; p<0.0001. enterokinase; 10 μg/mL, GM6001; 1 μM

FIG. 28 presents a chart showing exemplary cancers with aberrant Notch activity and protease overexpression.

FIGS. 29A and 29B present data showing anti-NRR1 scFv-NRR fusion can be used as a soluble probody for Notch signaling inhibition. (FIG. 29A) Schematic showing the NRR (arrow) as the inhibitory mask for the anti-NRR1 scFv (*) Notch1 signaling antagonist. Proteolytic processing is expected to dissociate the mask from the antibody fragment, which can then inhibit Notch1 signaling. (FIG. 29B) HEK293-FT cells were transiently transfected with pcDNA3-based plasmids encoding various pro-antibody domain constructs. Conditioned media was collect and cleaved with TEVp, enterokinase and pre-activated MMP2, and analyzed by western blot. Cleavage is shown to be specific to the intended substrate linker. (FIG. 29C) A CHO-K1 cell line expressing a chimeric Notch receptor (hN1 ECD-Gal4 ICD) with a UAS:H2B-Citrine reporter gene were plated on the indicated condition. At the time of plating, cells were treated either uncleaved or pre-cleaved pro-antibody fragments with TEVp. Cells were imaged 24 hours after plating.

FIGS. 30A and 30B present data showing anti-NRR1-based probodies can bind Notch1 receptors following deletion of the S1 loop (ΔS1) containing the NRR's furin cleavage site. The probodies used represent the anti-NRR1 scFv fused to with a soluble mutant NRR (V1667D) from Notch1, linked together via a TEV protease substrate sequence. The probodies were also fused with a HaloTag domain, which remains attached to the anti-NRR scFv following TEV cleavage. CHO-K1 cells expressing a Notch1-Gal4 chimeric receptor were treated with intact (uncleaved) and TEV protease-cleaved versions of the indicated probodies. The HaloTag domains were conjugated with a JFX650 dye to permit visualization of their binding to cell surface Notch1-Ga4 receptors. (FIG. 30A) Fluorescently labeled probody containing the S1 cleavage loop (Probody^{V1667D}-JF650) binds Notch1-Gal4 only in its TEV protease-cleaved state. (FIG. 30B) Fluorescently labeled probody containing the S1 cleavage loop deletion (Probody^{V1667D}(ΔS1)-JF650) similarly binds Notch1-Gal4 in a manner that is dependent on TEV protease cleavage. Insets represent emissions from a constitutively expressed H2B-mTurq2 expressed by the analyzed cells.

FIGS. 31A-31C present schematics and data showing activation of Notch1-Gal4 by immobilized DLL4 ligand is inhibited by TEV protease-mediated probody activation. (FIG. 31A) Cartoon of reporter CHO-K1 cells expressing a Notch1-Gal4 chimeric receptor and containing a Gal4-dependent fluorescent reporter gene (UAS-H2B-Citrine). (FIG. 31B) Cells are activated by grown on culture surfaces containing immobilized DLL4 ligand. Binding of the immobilize ligand to the Notch1-Gal4 receptor induces receptor processing events that lead to the liberation of the Gal4 intracellular domain (ICD) such that it can translocate to the nucleus to induce transcription and expression of the H2B-Citrine reporter. DLL4-mediated receptor activation can be blocked by treatment with a gamma secretase inhibitor (DAPT, to prevent ICD release); inhibition can also be achieved via the binding of a protease-activated probody to the Notch1 receptor Negative Regulatory Region (NRR). (FIG. 31C) Fluorescence emissions from the H2B-Citrine reporter overlaid with transmitted light images from the Notch1-Gal4 reporter cells. Cells were grown in microwells containing immobilized DLL4 under the indicated conditions and signaling levels were analyzed two days later. Images represent cells grown with DLL4 alone (left), with DLL4 and DAPT (second from left), with DLL4 and unactivated Probody^{V1667D} (third from left), or DLL4 and TEV protease-cleaved (activated) Probody^{V1667D} (right). Probodies represent the anti-NRR1 scFv fused with soluble mutant NRR (V1667D) from Notch1,as linked together via a TEV protease substrate sequence. When left unactivated (uncleaved), the probody cannot bind/inhibit Notch 1-Gal4 and thus DLL4 induced reporter gene action. When cleaved by TEV protease, the activated probody can bind the Notch1-Gal4 receptor's NRR and inhibits its activation by the DLL4 ligand (thus reducing signaling levels).

FIG. 32 presents data showing anti-NRR1-based probodies can bind Notch1 receptors following deletion of the S1 loop (ΔS1) containing the NRR's furin cleavage site. The probodies used represent the anti-NRR1 scFv fused to with a soluble mutant NRR (V1667D) from Notch1, linked together via a TEV protease substrate sequence. The probodies were also fused with a HaloTag domain, which remains attached to the anti-NRR scFv following TEV cleavage. CHO-K1 cells expressing a Notch1-Gal4 chimeric receptor were treated with intact (uncleaved) and TEV protease-cleaved versions of the indicated probodies. The HaloTag domains were conjugated with a JFX650 dye to permit visualization of their binding to cell surface Notch1-Ga4 receptors. (Left) Fluorescently labeled probody containing the S1 cleavage loop (Probody^{V1667D}-JF650) binds Notch1-Gal4 only in its TEV protease-cleaved state. (Right) Fluorescently labeled probody containing the S1 cleavage loop deletion (Probody^{V1667D}(ΔS1)-JF650) similarly binds Notch1-Gal4 in a manner that is dependent on TEV protease cleavage. Insets represent emissions from a constitutively expressed H2B-mTurq2 expressed by the analyzed cells.

FIGS. 33A-33C present schematics and data showing activation of a probody/masked antibody with specificity against human Notch1 (hNotch) NRR. (FIG. 33A) Schematic depicting the TEV protease (TEVp)-mediated activation and binding of the anti-hNotch1 probody. The probody sequence used contains the V1677D mutant NRR as a “mask” and is linked to an anti-hNotch1 scFv via a linker containing a TEVp substrate sequence. Upon cleavage by TEVp, the NRR mask dissociates from the scFv and is thus able to bind its corresponding antigen. A fluorophore is linked to the probody sequence to permit visualization. (FIGS. 33B and 33C) Tests of scFv binding to hNotch1. Clonal CHO-K1 cells expressing a stably integrated hNotch1 receptor were incubated with the body alone (FIG. 33B) or with the pro body treated with TEVp (FIG. 33C). Following a 40-minute incubation, probody-containing media was removed, and cells were washed with fresh media prior to fixation and DNA staining with Hoechst-JF526 (inset). Cells were imaged to detect cell-surface bound (activated) probody and Hoechst-JF526. Binding to hNotch1 was observed only with the TEVp-cleaved probody sequence. The antibody sequence used in the body can inhibit signaling by wild-type and various disease-associated hNotch1 mutations (Wu et al. Nature 464: 1052-1057 (2010)).

FIG. 34 presents data showing treatment with protease-activated probody reduces levels of cleaved Notch1 intracellular domain (NICD) in the human-derived T-cell acute lymphoblastic leukemia cell line HBP-ALL. HPB-ALL express a double mutant Notch1 receptor that is constitutively activated and by cells. These mutations are confined to the Notch1 NRR (p.Leu1574Pro) and NICD (p.Asp2442fs*39). As a result of these mutation, a cleaved NICD units are constitutively generated by HPB-ALL cells in a ligand-independent manner. HPB-ALL cells were treated with or without probodies based on the anti-NRR1 scFv fused to a soluble mutant NRR (V1667D) from Notch1, as linked via a TEV protease substrate sequence. After 40 hours of treatment, cells lysates were processed for western blotting using an antibody recognizing the cleaved Notch1 ICD (anti-Cleaved Notch1, New England Biolabs). Detection of anti-Cleaved Notch1 bands (top) showed that cleaved NICD levels were reduced in cells treated with TEV protease-activated probody, but not those treated with unactivated (intact) probody. Drug-untreated cells, or cells treated with the gamma secretase inhibitor DAPT, were processed and analyzed as controls. The membrane was stripped and re-blocked prior to loading control detection with an anti-GAPDH antibody (bottom).

FIGS. 35A and 35B present data showing Cleavage-Dependent Binding of an Anti-Notch2 and Anti-Notch3 Probodies to Receptors Expressed on Cell Surfaces. TEV protease-cleavable probodies based on (FIG. 35A) Anti-Notch2 and the Notch2 NRR (NRR2) or (FIG. 35B) Anti-Notch3 and the Notch3 NRR (NRR3) were tested against HEK293 cells expressing recombinant NRR2- and NRR3-containing receptors. Probodies were labeled with a JFX650 fluorescent dye. Cells were treated with uncleaved or TEV protease-cleaved probody solutions prior to the fixation and imaging of cells. Cells were co-transfected DNAs encoding tagBFP2 in combination with (FIG. 35A) an NRR2-containing receptor, or (FIG. 35B) an NRR3-containing receptor. Cells were stained with the indicated probodies 1 day after transfection. The anti-Notch2 probody is based on anti-NRR2 fused to NRR2, as connected via a TEV cleavable linker; the anti-Notch3 probody is based on anti-NRR3 fused to NRR3 via a TEV cleavable linker. Both probodies contained HaloTag domains fused to their scfv-based antibody domains.

FIGS. 36A and 36B present data showing Cleavage-Dependent Binding of a Brontictuzumab-Based Probody to Notch1 Receptors. A probody containing a brontictuzumab-based scFv was fused to a TEV cleavable linker and a soluble form of the human Notch1 NRR. Probodies containing either (FIG. 36A) the V1667D NRR mutant NRR, or (FIG. 36B) wild-type NRR were tested. The probodies also contained a HaloTag for labeling with a JFX650 dye. Binding of the probody to Notch1 receptors was tested using uncleaved and TEV protease-cleaved probody. The evaluated cells were based on CHO-K1 celle overexpressing a human Notch1-derived receptor; cells also expressed H2B-mTurq as a fluorescent marker (inset). Brontictuzumab is an antibody that binds the NRR from Notch1 (NRR1). Brontictuzumab (aka OMP-52M51) is originally from Oncomed Pharmaceuticals. This antibody is distinct from the Genentech anti-NRR1 used in our receptor and other Notch1 probody designs.

FIG. 37 present data showing Probody with a mask based on the wild-type Notch1-NRR binds Notch1 Receptors in a Cleavage-Dependent Manner. A probody containing the anti-NRR1 scFv was fused to a TEV protease cleavable linker and the wild-type Notch1 NRR. The probodies also contained a HaloTag for labeling with a JFX650 dye. Binding of the probody to Notch1 receptors was tested using uncleaved and TEV protease-cleaved probody. The evaluated cells were based on CHO-K1 celle overexpressing a human Notch1-derived receptor; cells also expressed H2B-mTurq as a fluorescent marker (inset).

FIG. 38A-38C present data showing MMP2/9-Pronotch and MMP14-ProNotch are cleaved and activated by enzymes secreted by cancer cells. (FIG. 38A) Schematic depicting the ProNotch design. A protease substrate is used to link the NRR with an anti-NRR scFv. When cleaved by a corresponding protease, the scFv dissociates from the receptor, and the mutated NRR then undergoes ligand-independent activation, leading to the activation and transcription of a target gene. (FIG. 38B-38C) Fluorescence images of HEK293 reporter cells (UAS-H2B-mCherry) expressing ProNotchT2A-tagBFP2 as grown with HT1080 fibrosarcoma cells. The HEK293 reporter cells express the indicated receptors; HT1080 cells express MMP-2, -9, and -14 (cells were treated with 100 nM PMA). HEK293 cells do not express MMP-14. Fluorescence emissions were collected after 48 hours of growth in coculture. Emissions from tagBFP2 indicate receptor-expressing cells; emissions from H2B-mCherry indicate signaling-mediated activation of the UAS-H2B-mCherry reporter gene. Scale bar 500 um.

FIG. 39 present data showing Renin-cleavable ProNotch can be activated by renin protease. Fluorescence images of U2OS reporter cells (UAS-DsRed-Express2) expressing a renin-cleavable ProNotch receptor containing a Gal4VP64 intracellular domain (renin-Pronotch-Gal4VP64). Cells were transduced with lentiviral constructs encoding renin-ProNotch-Gal4VP64. Two following transduction cells were exchanged into conditioned control media (left) or conditioned media containing active renin protease (right). Expression of the DsRed reporter was visualized by fluorescence microscopy two days later. Conditioned media solutions were collected from cultures containing control HEK293-FT cells or HEK293-FT cells transfected with DNA encoding a secreted renin protease. A constitutively activated secreted renin containing a furin cleavage site was utilized. Scale bars are 500 microns.

FIG. 40A-40B present data showing Pronotch receptors containing a brontictuzumab-based scFv are expressed and present on the cell surface of transfected cells. Cell surface detection of expression and presentation of ProNotch receptors containing a brontictuzumab-based NRR-binding scFv. The indicated receptor constructs were expressed in HEK293-FT cells by transient transfection. Surface-localized receptor copies were detected by staining live cultures with a cell-impermeant fluorescent HaloTag ligand (HaloLigand-JF635i). Excess dye was removed, and cells were washed before recording emissions from the covalently dye-labeled surface receptors by fluorescence microscopy. Insets represent emissions from a co-expressed T2A-tagBFP2. Scale bars are 200 microns.

### DETAILED DESCRIPTION

Disclosed herein, in part, is a modular transcriptional switch that activates in response to extracellular proteolysis by taking inspiration from prodrug design and the activation mechanism of cancerous forms of Notch. By incorporating an anti-NRR1 scFv in a SynNotch receptor containing a hyperactive Notch mutation with protease-cleavable substrate, an autoinhibited signaling domain that is sensitive to proteolysis and results in ligand-independent activation was constructed. Inventors have rewired the autoinhibitory function of the NRR to be sensitive to protease compared to natural Notch which requires ligand transendocytosis. Several of the designs disclosed herein were found to have low basal activation and high protease-induced activation. Interestingly, the activity of these receptors correlated with the mutated NRR's characterized in vitro destabilization. It was found that once the inhibitory mask is cleaved, the receptor undergoes similar proteolytic cleavage events as Notch, suggesting these receptors could potentially function in the several cell types SynNotch have been applied to including neurons and immune cells. Inventors took advantage of the receptor's conserved proteolytic release mechanism to allow the ICD to be exchanged in addition to the protease substrate which enabled customizable input-output signaling pathways. It is demonstrated herein these receptors function in a synthetic biology application by inducing myogenic differentiation in C3H10T1/2 multipotent fibroblasts. It was additionally found these receptors to be sensitive to endogenously expressed protease activity in coculture. Results disclosed herein indicated the synthetic receptor designs described herein can be used to sense and respond to proteolysis within the body, such as in diseased tissues.

Accordingly, one aspect provided herein is a synthetic nucleic acid encoding a mutant Notch NRR (Negative Regulatory Region), wherein the mutant Notch NRR comprises a mutation that induces ligand-independent activation of regulated intramembrane proteolysis resulting to Notch signaling, SynNotch signaling, or at least another signaling outcome.

Also provided herein is mutant Notch NRR polypeptide encodes by any of the synthetic nucleic acid encoding a mutant Notch NRR. In one embodiment, the mutant Notch NRR polypeptide comprises a sequence of SEQ ID NO: 1 or 2.

In one embodiment, the mutant Notch NRR polypeptide comprises a sequence having at least 50%, 60%, 70%, 80%, 90%, 91%, 92%, 93%, 94%, 95%, 96%, 97%, 98%, or 99% sequence identify to SEQ ID NO: 1 or 2.

Another aspect provided herein is a fusion protein comprising: i) a Notch NRR (Negative Regulatory Region)-binding antibody, and ii) a mutant Notch NRR comprising a mutation that induces ligand-independent activation of regulated intramembrane proteolysis resulting to Notch signaling, SynNotch signaling, or at least another signaling outcome.

In one embodiment, the Notch NRR-binding antibody is further fused to a transmembrane domain. For example, in one embodiment, the Notch NRR-binding antibody is a Notch NRR-binding scFv and the Notch NRR-binding scFv is fused to a transmembrane domain.

In one embodiment, wherein the fusion protein further comprises, positioned in between i) and ii), a linker.

In one embodiment, the Notch NRR-binding antibody further comprises a linker. For example, in one embodiment, the Notch NRR-binding antibody is a Notch NRR-binding scFv and the Notch NRR-binding scFv further comprises a linker, e.g., positioned between the heavy and light chain.

In one embodiment, the fusion protein further comprising a signal sequence at its N-terminal.

Another aspect provided herein is a fusion protein comprising: i) a Notch NRR (Negative Regulatory Region)-binding scFv, ii) a linker, and iii) a mutant Notch NRR, mutant Notch NRR comprises a mutation that induces ligand-independent activation of regulated intramembrane proteolysis resulting to Notch signaling, SynNotch signaling, or at least another signaling outcome.

Another aspect provided herein is a synthetic Notch receptor protein comprising, from N-terminal to C-terminal and in covalent linkage, (i) a scFv that binds to an at least one Notch NRR, (ii) a linker, (iii) a mutated Notch NRR, wherein the mutant Notch NRR comprises a mutation that induces ligand-independent activation of regulated intramembrane proteolysis resulting to Notch signaling, SynNotch signaling, or at least another signaling outcome, (iv) a transmembrane domain, and (v) an intracellular domain.

As used herein, a “fusion protein” refers to a non-naturally occurring protein or polypeptide having a desired function for use in the compositions and methods described herein. Such synthetic proteins can comprise one or more domains from or derived from a naturally occurring protein in combination with one or more domains from or derived from another naturally occurring protein to create a synthetic protein having desirable functions that are not found together naturally. Such domains include naturally occurring domains, as well as mutated or engineered domains derived from naturally occurring domains, or portions of a naturally occurring domain having a desired activity. For example, in some embodiments, a synthetic protein comprises one or more Notch Regulatory Region (NRR)-binding domains. Other examples of domains that can be used in the synthetic proteins described herein include transcriptional activation domains, transcriptional repressor domains, DNA-binding domains, such as zinc-finger-binding domains, protease domains, and the like. Other domains contemplated for use in the synthetic proteins described herein include extracellular domains, such as ligand-binding extracellular domains, transmembrane domains, and intracellular domains, such as intracellular signaling domains. In addition, the nucleic acid sequences encoding the synthetic proteins described herein can comprise additional sequence elements such as signal sequences and tag sequences.

In some embodiments of the synthetic proteins described herein, one or more additional “fusion” domains can be added to the synthetic protein to provide additional desired functionality. Well known examples of fusion domains include, but are not limited to, polyhistidine, Glu-Glu, glutathione S transferase (GST), thioredoxin, protein A, protein G, an immunoglobulin heavy chain constant region (Fc), maltose binding protein (MBP), or human serum albumin. A fusion domain can be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS6 (SEQ ID NO: 40)) fusion partners. As another example, a fusion domain can be selected so as to facilitate detection of the synthetic proteins. Examples of such detection domains include the various fluorescent proteins (e.g., GFP) as well as “epitope tags,” which are usually short peptide sequences for which a specific antibody is available. Well known epitope tags for which specific monoclonal antibodies are readily available include FLAG, influenza virus haemagglutinin (HA), and c-myc tags. Non-limiting tag sequences that can be used in the synthetic proteins described herein include SEQ ID NOs: 5-7 and 33. In some cases, the fusion domains have a protease cleavage site, such as for Factor Xa or Thrombin, which allows the relevant protease to partially digest the fusion proteins and thereby liberate the recombinant proteins therefrom. The liberated proteins can then be isolated from the fusion domain by subsequent chromatographic separation. Other types of fusion domains that can be selected include multimerizing (e.g., dimerizing, tetramerizing) domains and functional domain

Yet, another aspect provided herein is a protease regulatable Notch signaling inhibitor, the inhibitor comprising an anti-NRR antibody fused to a mutant NRR, having a protease cleavable linker positioned between the anti-NRR antibody and mutant NRR.

In one embodiment, the protease regulatable Notch signaling inhibitor further comprises at least one AND-gate protein. In one embodiment, the at least one AND-gate protein provides independent and/or distinct cleavage sites required to make the antigen binding site of the anti-NRR antibody available.

Yet, another aspect provided herein is a protease regulatable Notch signaling inhibitor, the inhibitor comprising an anti-NRR antibody fused to a transmembrane domain, and a mutant NRR, having a protease cleavable linker positioned between the anti-NRR antibody fused to the transmembrane domain and mutant NRR.

In one embodiment, cleavage of the protease cleavable linker result in dissociation of the mutant NRR and the anti-NRR antibody fused to a transmembrane domain. This dissociation results in inhibition of Notch signaling via binding of the anti-NRR antibody fused to a transmembrane domain to a receptor.

Finally, another aspect disclosed herein is a AND-gate protein-synthetic Notch receptor comprising: a signal sequence; a ligand binding domain (such as a tumor antigen); a NRR domain; a cleavable linker; a NRR-binding scFv; a Notch1 TMD; and an intracellular domain. In one embodiment, the AND-gate protein-synthetic Notch receptor comprising, from 5′ to 3′, a signal sequence; a ligand binding domain (such as a tumor antigen); a NRR domain; a cleavable linker; a NRR-binding scFv; a Notch1 TMD; and an intracellular domain.

“AND” gates as described herein include where two or more inputs are required for propagation of a signal. For example, in some instances, an AND gate allows signaling through two or more engineered receptors or portions thereof where two inputs, e.g., two ligands, are required for signaling through the two or more engineered receptors or portions thereof.

The gate protein-synthetic Notch receptor of claim 57, wherein the ICD is an ICD derived from Gal4, TetR, zinc fingers, Cas9, Cre recombinase, the Notch ICD (NICD), a dominant negative MAML (dnMAML), an inhibitor of transcriptional activity, and a protein with a nucleus-specific activity

In one embodiment, the ICD is an ICD derived from Gal4, TetR, zinc fingers, Cas9, Cre recombinase, the Notch ICD (NICD), a dominant negative MAML (dnMAML), an inhibitor of transcriptional activity, and a protein with a nucleus-specific activity.

In one embodiment, the protease regulatable Notch modulator has a sequence selected from SEQ ID NOs 29-34.

TEV-cleavable Anti-Notch1 probody, Genentech scFv (HaloTag-anti-NRR1-scFv-TEVcs-
msNRR1^{V1677D}):
(SEQ ID NO: 29)
MALPVTALLLPLALLLHAARPYPYDVPDYATGMAEIGTGFPFDPHYVEVLGERMHYVD
VGPRDGTPVLFLHGNPTSSYVWRNIIPHVAPTHRCIAPDLIGMGKSDKPDLGYFFDDHVRFMDA
FIEALGLEEVVLVIHDWGSALGFHWAKRNPERVKGIAFMEFIRPIPTWDEWPEFARETFQAFRTT
DVGRKLIIDQNVFIEGTLPMGVVRPLTEVEMDHYREPFLNPVDREPLWRFPNELPIAGEPANIVA
LVEEYMDWLHQSPVPKLLFWGTPGVLIPPAEAARLAKSLPNCKAVDIGPGLNLLQEDNPDLIGS
EIARWLSTLEISGGGGGSTGDGGGGEVQLVESGGGLVQPGGSLRLSCAASGFTFSSYWIHWVRQ
APGKGLEWVARINPPNRSNQYADSVKGRFTISADTSKNTAYLQMNSLRAEDTAVYYCARGSGF
RWVMDYWGQGTLVTVSSGGSSRSSSSGGGGSGGGGDIQMTQSPSSLSASVGDRVTITCRASQD
VSTAVAWYQQKPGKAPKLLIYSASFLYSGVPSRFSGSGSGTDFTLTISSLQPEDFATYYCQQFYT
TPSTFGQGTKVEIKGSNSGGTLEGGGSGENLYFQGGGGGRTGGGSEQKLISEEDLGGGSILDYSF
TGGAGRDIPPPQIEEACELPECQVDAGNKVCNLQCNNHACGWDGGDCSLNFNDPWKNCTQSLQ
CWKYFSDGHCDSQCNSAGCLFDGFDCQLTEGQCNPLYDQYCKDHFSDGHCDQGCNSAECEWD
GLDCAEHVPERLAAGTLVLVVLLPPDQLRNNSFHFLRELSHVLHTNVVFKRDAQGQQMIFPYY
GHEEELRKHPIKRSTVGWATSSLLPGTSGGRQRRELDPMDIRGSIDYLEIDNRQCVQSSSQCFQS
ATDVAAFLGALASLGSLNIPYKIEAVKSEPVEGPV*
TEV-cleavable Anti-Notch1 probody, Genentech scFv, S1 loop deletion (Halo Tag-anti-
NRR1-scFv-TEVcs-msNRR1^{V1677D}-deltaS1):
(SEQ ID NO: 30)
MALPVTALLLPLALLLHAARPYPYDVPDYATGMAEIGTGFPFDPHYVEVLGERMHYVD
VGPRDGTPVLFLHGNPTSSYVWRNIIPHVAPTHRCIAPDLIGMGKSDKPDLGYFFDDHVRFMDA
FIEALGLEEVVLVIHDWGSALGFHWAKRNPERVKGIAFMEFIRPIPTWDEWPEFARETFQAFRTT
DVGRKLIIDQNVFIEGTLPMGVVRPLTEVEMDHYREPFLNPVDREPLWRFPNELPIAGEPANIVA
LVEEYMDWLHQSPVPKLLFWGTPGVLIPPAEAARLAKSLPNCKAVDIGPGLNLLQEDNPDLIGS
EIARWLSTLEISGGGGGSTGDGGGGEVQLVESGGGLVQPGGSLRLSCAASGFTFSSYWIHWVRQ
APGKGLEWVARINPPNRSNQYADSVKGRFTISADTSKNTAYLQMNSLRAEDTAVYYCARGSGF
RWVMDYWGQGTLVTVSSGGSSRSSSSGGGGSGGGGDIQMTQSPSSLSASVGDRVTITCRASQD
VSTAVAWYQQKPGKAPKLLIYSASFLYSGVPSRFSGSGSGTDFTLTISSLQPEDFATYYCQQFYT
TPSTFGQGTKVEIKGSNSGGTLEGGGSGENLYFQGGGGGRTGGGSEQKLISEEDLGGGSILDYSF
TGGAGRDIPPPQIEEACELPECQVDAGNKVCNLQCNNHACGWDGGDCSLNFNDPWKNCTQSLQ
CWKYFSDGHCDSQCNSAGCLFDGFDCQLTEGQCNPLYDQYCKDHFSDGHCDQGCNSAECEWD
GLDCAEHVPERLAAGTLVLVVLLPPDQLRNNSFHFLRELSHVLHTNVVFKRDAQGQQMIFPYY
GMDIRGSIDYLEIDNRQCVQSSSQCFQSATDVAAFLGALASLGSLNIPYKIEAVKSEPVEGPV*
TEV-cleavable Anti-Notch1 probody, Genentech scFv, Wild-type NRR (HaloTag-anti-
NRR1-scFv-TEVcs-msNRR1^{WT}):
(SEQ ID NO: 31)
MALPVTALLLPLALLLHAARPYPYDVPDYATGMAEIGTGFPFDPHYVEVLGERMHYVD
VGPRDGTPVLFLHGNPTSSYVWRNIIPHVAPTHRCIAPDLIGMGKSDKPDLGYFFDDHVRFMDA
FIEALGLEEVVLVIHDWGSALGFHWAKRNPERVKGIAFMEFIRPIPTWDEWPEFARETFQAFRTT
DVGRKLIIDQNVFIEGTLPMGVVRPLTEVEMDHYREPFLNPVDREPLWRFPNELPIAGEPANIVA
LVEEYMDWLHQSPVPKLLFWGTPGVLIPPAEAARLAKSLPNCKAVDIGPGLNLLQEDNPDLIGS
EIARWLSTLEISGGGGGSTGDGGGGEVQLVESGGGLVQPGGSLRLSCAASGFTFSSYWIHWVRQ
APGKGLEWVARINPPNRSNQYADSVKGRFTISADTSKNTAYLQMNSLRAEDTAVYYCARGSGF
RWVMDYWGQGTLVTVSSGGSSRSSSSGGGGSGGGGDIQMTQSPSSLSASVGDRVTITCRASQD
VSTAVAWYQQKPGKAPKLLIYSASFLYSGVPSRFSGSGSGTDFTLTISSLQPEDFATYYCQQFYT
TPSTFGQGTKVEIKGSNSGGTLEGGGSGENLYFQGGGGGRTGGGSEQKLISEEDLGGGSILDYSF
TGGAGRDIPPPQIEEACELPECQVDAGNKVCNLQCNNHACGWDGGDCSLNFNDPWKNCTQSLQ
CWKYFSDGHCDSQCNSAGCLFDGFDCQLTEGQCNPLYDQYCKDHFSDGHCDQGCNSAECEWD
GLDCAEHVPERLAAGTLVLVVLLPPDQLRNNSFHFLRELSHVLHTNVVFKRDAQGQQMIFPYY
GHEEELRKHPIKRSTVGWATSSLLPGTSGGRQRRELDPMDIRGSIVYLEIDNRQCVQSSSQCFQS
ATDVAAFLGALASLGSLNIPYKIEAVKSEPVEGPV*
TEV-cleavable Anti-Notch1 probody, with brontictuzumab scFv, Wild-type NRR
(HaloTag-brontictuzumab-scFv-TEVcs-hNRR1^{WT}):
(SEQ ID NO: 32)
MALPVTALLLPLALLLHAARPYPYDVPDYATGMAEIGTGFPFDPHYVEVLGERMHYVD
VGPRDGTPVLFLHGNPTSSYVWRNIIPHVAPTHRCIAPDLIGMGKSDKPDLGYFFDDHVRFMDA
FIEALGLEEVVLVIHDWGSALGFHWAKRNPERVKGIAFMEFIRPIPTWDEWPEFARETFQAFRTT
DVGRKLIIDQNVFIEGTLPMGVVRPLTEVEMDHYREPFLNPVDREPLWRFPNELPIAGEPANIVA
LVEEYMDWLHQSPVPKLLFWGTPGVLIPPAEAARLAKSLPNCKAVDIGPGLNLLQEDNPDLIGS
EIARWLSTLEISGGGGGSTGDGGGGQVQLVQSGAEVKKPGASVKISCKVSGYTLRGYWIEWVR
QAPGKGLEWIGQILPGTGRTNYNEKFKGRVTMTADTSTDTAYMELSSLRSEDTAVYYCARFDG
NYGYYAMDYWGQGTTVTVSSGGSSRSSSSGGGGSGGGGQAVVTQEPSLTVSPGGTVTLTCRSS
TGAVTTSNYANWFQQKPGQAPRTLIGGTNNRAPGVPARFSGSLLGGKAALTLSGAQPEDEAEY
YCALWYSNHWVFGGGTKLTVLGSNSGGTLEGGGSGENLYFQGGGGGRTGGGSEQKLISEEDL
GGGSILDYSFGGGAGRDIPPPLIEEACELPECQEDAGNKVCSLQCNNHACGWDGGDCSLNFNDP
WKNCTQSLQCWKYFSDGHCDSQCNSAGCLFDGFDCQRAEGQCNPLYDQYCKDHFSDGHCDQ
GCNSAECEWDGLDCAEHVPERLAAGTLVVVVLMPPEQLRNSSFHFLRELSRVLHTNVVFKRDA
HGQQMIFPYYGREEELRKHPIKRAAEGWAAPDALLGQVKASLLPGGSEGGRRRRELDPMDVRG
SIVYLEIDNRQCVQASSQCFQSATDVAAFLGALASLGSLNIPYKIEAVQSETVEPGSLVPRGSSHH
HHHHGPV*
Anti-Notch2 probody (HaloTag-anti-NRR2-scFv-TEVcs-msNRR2^{WT});
(SEQ ID NO: 33)
MALPVTALLLPLALLLHAARPYPYDVPDYATGMAEIGTGFPFDPHYVEVLGERMHYVD
VGPRDGTPVLFLHGNPTSSYVWRNIIPHVAPTHRCIAPDLIGMGKSDKPDLGYFFDDHVRFMDA
FIEALGLEEVVLVIHDWGSALGFHWAKRNPERVKGIAFMEFIRPIPTWDEWPEFARETFQAFRTT
DVGRKLIIDQNVFIEGTLPMGVVRPLTEVEMDHYREPFLNPVDREPLWRFPNELPIAGEPANIVA
LVEEYMDWLHQSPVPKLLFWGTPGVLIPPAEAARLAKSLPNCKAVDIGPGLNLLQEDNPDLIGS
EIARWLSTLEISGGGGGSTGDGGGGEVQLVESGGGLVQPGGSLRLSCAASGYTFSSYGMSWVR
QAPGKGLEWVSYIYPYSGATYYADSVKGRFTISADTSKNTAYLQMNSLRAEDTAVYYCARHSG
YYRISSAMDVWGQGTLVTVSAGGSSRSSSSGGGGSGGGGDIQMTQSPSSLSASVGDRVTITCRA
SQNIKRFLAWYQQKPGKAPKLLIYGASTRESGVPSRFSGSGSGTDFTLTISSLQPEDFATYYCQQ
YYRSPHTFGQGTKVEIKRGSNSGGTLEGGGSGENLYFQGGGGGRTGGGSEQKLISEEDLGGGSL
YTAPTSTPPATCQSQYCADKARDGICDEACNSHACQWDGGDCSLTMEDPWANCTSTLRCWEYI
NNQCDEQCNTAECLFDNFECQRNSKTCKYDKYCADHFKDNHCDQGCNSEECGWDGLDCASD
QPENLAEGTLIIVVLLPPEQLLQDSRSFLRALGTLLHTNLRIKQDSQGALMVYPYFGEKSAAMKK
QKMTRRSLPEEQEQEQEVIGSKIFLEIDNRQCVQDSDQCFKNTDAAAALLASHAIQGTLSYPLVS
VFSEPVEGPV*
Anti-Notch3 probody, with anti-NRR3(i) scfv (HaloTag-anti-NRR3(i)-scFv-TEVcs-
hNRR3^{WT}):
(SEQ ID NO: 34)
MALPVTALLLPLALLLHAARPYPYDVPDYATGMAEIGTGFPFDPHYVEVLGERMHYVD
VGPRDGTPVLFLHGNPTSSYVWRNIIPHVAPTHRCIAPDLIGMGKSDKPDLGYFFDDHVRFMDA
FIEALGLEEVVLVIHDWGSALGFHWAKRNPERVKGIAFMEFIRPIPTWDEWPEFARETFQAFRTT
DVGRKLIIDQNVFIEGTLPMGVVRPLTEVEMDHYREPFLNPVDREPLWRFPNELPIAGEPANIVA
LVEEYMDWLHQSPVPKLLFWGTPGVLIPPAEAARLAKSLPNCKAVDIGPGLNLLQEDNPDLIGS
EIARWLSTLEISGGGGGSTGDGGGGEVQLVESGGGLVQPGGSLRLSCAASGYAFTDYWMTWVR
QAPGKGLEWVAEISPNSGGTNFNEKFKGRFTISVDNAKNSLYLQMNSLRAEDTAVYYCARGEIR
YNWFAYWGQGTLVTVSSGGSSRSSSSGGGGSGGGGDIQMTQSPSSLSASVGDRVTITCKASQNV
GNNIAWYQQKPGKAPKLLIYYASNRYTGVPSRFSGSGYGTDFTLTISSLQPEDFATYYCQRLYNS
PFTFGGGTKVEIKGSNSGGTLEGGGSGENLYFQGGGGGRTGGGSEQKLISEEDLGGGSAPAAAP
EVSEEPRCPRAACQAKRGDQRCDRECNSPGCGWDGGDCSLSVGDPWRQCEALQCWRLFNNSR
CDPACSSPACLYDNFDCHAGGRERTCNPVYEKYCADHFADGRCDQGCNTEECGWDGLDCASE
VPALLARGVLVLTVLLPPEELLRSSADFLQRLSAILRTSLRFRLDAHGQAMVFPYHRPSPGSEPR
ARRELAPEVIGSVVMLEIDNRLCLQSPENDHCFPDAQSAADYLGALSAVERLDFPYPLRDVRGE
PVEGPV*

In one embodiment, the protease regulatable modulator inhibits Notch signaling upon binding of, e.g., the Notch receptor. In one embodiment, the protease regulatable modulator inhibits Notch signaling upon binding of, e.g., the Notch receptor, by at least 10%. In one embodiment, the protease regulatable modulator inhibits Notch signaling upon binding of, e.g., the Notch receptor, by 10% or greater, 15% or greater 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 98% or greater, 99% or greater, or complete or 100% as compared to an appropriate control, e.g., the level prior to binding of, e.g., the Notch receptor.

In one embodiment, the protease regulatable modulator activates Notch signaling upon binding of, e.g., the Notch receptor. In one embodiment, the protease regulatable modulator activates Notch signaling upon binding of, e.g., the Notch receptor, by at least 10%. In one embodiment, the protease regulatable modulator activates Notch signaling upon binding of, e.g., the Notch receptor, by 10% or greater, 15% or greater 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 98% or greater, 99% or greater, or 100%, 200%, 300%, 500%, 1000%, 1500%, 2000%, 3000%, or greater as compared to an appropriate control, e.g., the level prior to binding of, e.g., the Notch receptor.

In one embodiment, the modulator is further conjugated to a drug, forming an antibody-drug conjugate. Drug conjugates should be attached to or near the anti-NRR region of the protease regulatable Notch modulator. Exemplary drugs that can be conjugated include, but are not limited to, tubulin binders (e.g, Auristatins, including monomethyl auristatin E (MMAE), monomethyl auristatin F (MMAF), eribulin and mertansine (DM1); Topoisomerase inhibitors (e.g., SN-38 and Dx8951 (exatecan)); DNA binders (e.g., Calicheamicin, Pyrrolobenzodiazepines (PBDs), Duocarmycins, including CC-1065); and Amanatins (e.g., α-amanitin, β-amanitin, HDP-101).

In one embodiment, the modulator is used for delivery of cargo. Exemplary cargo include lipid nucleic acids, nanoparticles or virus-like particles, e.g., for uptake by Notch-expressing cells in a protease-cleavage-dependent manner. In one embodiment, delivery of the cargo to specific cell types, select tissues, or diseased areas.

In one embodiment, the protease regulatable binds a receptor having a sequence of SEQ ID NOs 35-38. One aspect of herein describes a receptor having a sequence of SEQ ID NOs 35-38.

(SEQ ID NO: 35)

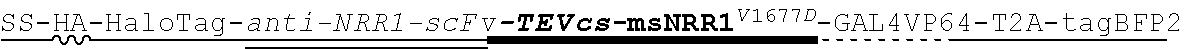

MALPVTALLLPLALLLHAARPYPYDVPDYATGMAEIGTGFPFDPHYVEVLGERMHYVD
VGPRDGTPVLFLHGNPTSSYVWRNIIPHVAPTHRCIAPDLIGMGKSDKPDLGYFFDDHVRFMDA
FIEALGLEEVVLVIHDWGSALGFHWAKRNPERVKGIAFMEFIRPIPTWDEWPEFARETFQAFRTT
DVGRKLIIDQNVFIEGTLPMGVVRPLTEVEMDHYREPFLNPVDREPLWRFPNELPIAGEPANIVA
LVEEYMDWLHQSPVPKLLFWGTPGVLIPPAEAARLAKSLPNCKA VDIGPGLNLLQEDNPDLIGS
EIARWLSTLEISGGGGGSTGDGGGGEVQLVESGGGLVQPGGSLRLSCAASGFTFSSYWIHWVRQAP
GKGLEWVARINPPNRSNOYADSVKGRFTISADTSKNTAYLOMNSLRAEDTAVYYCARGSGERWVMDY
WGQGTLVTVSSGGSSRSSSSGGGGSGGGGDIQMTQSPSSLSASVGDRVTITCRASQDVSTAVAWYQQK
PGKAPKLLIYSASFLYSGVPSRFSGSGSGTDFTLTISSLQPEDFATYYCQQFYTTPSTFGQGTKVEIKGS
NSGGTLEGGGSGENLYFQGGGGGRTGGGSEQKLISEEDLGGSILDYSFTGGAGRDIPPPQIEE
ACELPECQVDAGNKVCNLQCNNHACGWDGGDCSLNFNDPWKNCTQSLQCWKYFSDGHC
DSQCNSAGCLFDGFDCQLTEGQCNPLYDQYCKDHFSDGHCDQGCNSAECEWDGLDCAEH
VPERLAAGTLVLVVLLPPDQLRNNSFHFLRELSHVLHTNVVFKRDAQGQQMIFPYYGHEE
ELRKHPIKRSTVGWATSSLLPGTSGGRQRRELDPMDIRGSIDYLEIDNRQCVQSSSQCFQSA
TDVAAFLGALASLGSLNIPYKIEAVKSEPVEPPLPSQLHLMYVAAAAFVLLFFVGCGVLLSR

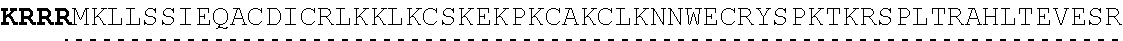

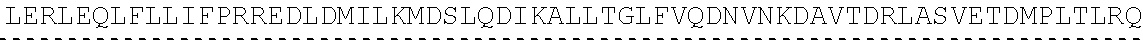

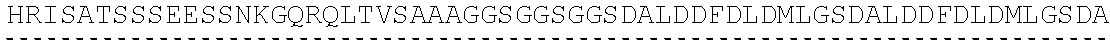

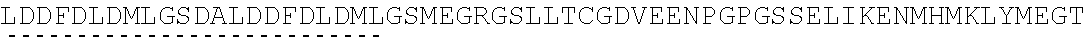

VDNHHFKCTSEGEGKPYEGTQTMRIKVVEGGPLPFAFDILATSFLYGSKTFINHTQGIPDFFKQSF
PEGFTWERVTTYEDGGVLTATQDTSLQDGCLIYNVKIRGVNFTSNGPVMQKKTLGWEAFTETL
YPADGGLEGRNDMALKLVGGSHLIANIKTTYRSKKPAKNLKMPGVYYVDYRLERIKEANNETY
VEQHEVAVARYCDLPSKLGHKLN
(SEQ ID NO: 36)

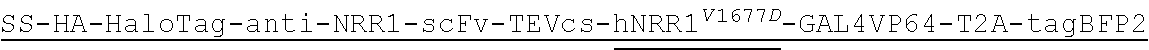

MALPVTALLLPLALLLHAARPYPYDVPDYATGMAEIGTGFPFDPHYVEVLGERMHYVDVGPRD
GTPVLFLHGNPTSSYVWRNIIPHVAPTHRCIAPDLIGMGKSDKPDLGYFFDDHVRFMDAFIEALG
LEEVVLVIHDWGSALGFHWAKRNPERVKGIAFMEFIRPIPTWDEWPEFARETFQAFRTTDVGRK
LIIDQNVFIEGTLPMGVVRPLTEVEMDHYREPFLNPVDREPLWRFPNELPIAGEPANIVALVEEY
MDWLHQSPVPKLLFWGTPGVLIPPAEAARLAKSLPNCKAVDIGPGLNLLQEDNPDLIGSEIARW
LSTLEISGGGGGSTGDGGGGEVOLVESGGGLVQPGGSLRLSCAASGFTFSSYWIHWVRQAPGKG
LEWVARINPPNRSNQYADSVKGRFTISADTSKNTAYLQMNSLRAEDTAVYYCARGSGFRWVM
DYWGQGTLVTVSSGGSSRSSSSGGGGSGGGGDIQMTQSPSSLSASVGDRVTITCRASQDVSTAV
AWYQQKPGKAPKLLIYSASFLYSGVPSRFSGSGSGTDFTLTISSLQPEDFATYYCQQFYTTPSTFG
QGTKVEIKLEGGGSGENLYFOSGRTGGGSEQKLISEEDLGGGSILDYSFGGGAGRDIPPPLIEEAC
ELPECQEDAGNKVCSLOCNNHACGWDGGDCSLNFNDPWKNCTOSLOCWKYESDGHCDSQCN
SAGCLEDGEDCORAEGOCNPLYDQYCKDHESDGHCDOGCNSAECEWDGLDCAEHVPERLAAG
TLVVVVLMPPEQLRNSSFHELRELSRVLHTNVVFKRDAHGQQMIFPYYGREEELRKHPIKRAAE
GWAAPDALLGQVKASLLPGGSEGGRRRRELDPMDVRGSIDYLEIDNROCVQASSQCFQSATDV
AAFLGALASLGSLNIPYKIEAVQSETVEPPPPAQLHEMYVAAAAFVLLFFVGCGVLLSRKRRRM
KLLSSIEQACDICRLKKLKCSKEKPKCAKCLKNNWECRYSPKTKRSPLTRAHLTEVESRLERLEQ
LFLLIFPREDLDMILKMDSLQDIKALLTGLFVQDNVNKDAVTDRLASVETDMPLTLRQHRISATS
SSEESSNKGQRQLTVSAAAGGSGGSGGSDALDDFDLDMLGSDALDDFDLDMLGSDALDDFDLD
MLGSDALDDFDLDMLGSMEGRGSLLTCGDVEENPGPGSSELIKENMHMKLYMEGTVDNHHFK
CTSEGEGKPYEGTQTMRIKVVEGGPLPFAFDILATSFLYGSKTFINHTQGIPDFFKQSFPEGFTWE
RVTTYEDGGVLTATQDTSLQDGCLIYNVKIRGVNFTSNGPVMQKKTLGWEAFTETLYPADGGL
EGRNDMALKLVGGSHLIANIKTTYRSKKPAKNLKMPGVYYVDYRLERIKEANNETYVEQHEVA
VARYCDLPSKLGHKLN*
(SEQ ID NO: 37)

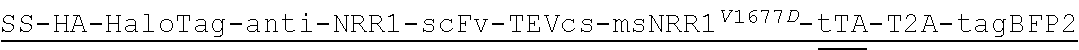

MALPVTALLLPLALLLHAARPYPYDVPDYATGMAEIGTGFPFDPHYVEVLGERMHYVD
VGPRDGTPVLFLHGNPTSSYVWRNIIPHVAPTHRCIAPDLIGMGKSDKPDLGYFFDDHVRFMDA
FIEALGLEEVVLVIHDWGSALGFHWAKRNPERVKGIAFMEFIRPIPTWDEWPEFARETFQAFRTT
DVGRKLIIDQNVFIEGTLPMGVVRPLTEVEMDHYREPFLNPVDREPLWRFPNELPIAGEPANIVA
LVEEYMDWLHQSPVPKLLFWGTPGVLIPPAEAARLAKSLPNCKAVDIGPGLNLLQEDNPDLIGS
EIARWLSTLEISGGGGGSTGDGGGGEVOLVESGGGLVQPGGSLRLSCAASGFTFSSYWIHWVRQ
APGKGLEWVARINPPNRSNQYADSVKGRFTISADTSKNTAYLQMNSLRAEDTAVYYCARGSGF
RWVMDYWGQGTLVTVSSGGSSRSSSSGGGGSGGGGDIQMTQSPSSLSASVGDRVTITCRASQD
VSTAVAWYQQKPGKAPKLLIYSASFLYSGVPSRFSGSGSGTDFTLTISSLQPEDFATYYCQQFYT
TPSTFGQGTKVEIKGSNSGGTLEGGGSGENLYFOGGGGGRTGGGSEQKLISEEDLGGGSILDYSF
TGGAGRDIPPPQIEEACELPECQVDAGNKVCNLQCNNHACGWDGGDCSLNFNDPWKNCTQSLQ
CWKYFSDGHCDSQCNSAGCLFDGFDCQLTEGQCNPLYDQYCKDHFSDGHCDQGCNSAECEWD
GLDCAEHVPERLAAGTLVLVVLLPPDQLRNNSFHFLRELSHVLHTNVVFKRDAQGQQMIFPYY
GHEEELRKHPIKRSTVGWATSSLLPGTSGGRQRRELDPMDIRGSIDYLEIDNRQCVQSSSQCFQS
ATDVAAFLGALASLGSLNIPYKIEAVKSEPVEPPLPSQLHLMYVAAAAFVLLFFVGCGVLLSRKR
RRQLCIQKLMSRLDKSKVINSALELLNEVGIEGLTTRKLAQKLGVEQPTLYWHVKNKRALLDAL
AIEMLDRHHTHFCPLEGESWQDFLRNNAKSFRCALLSHRDGAKVHLGTRPTEKQYETLENQLAF
LCQQGFSLENALYALSAVGHFTLGCVLEDQEHQVAKEERETPTTDSMPPLLRQAIELFDHOGAE
PAFLFGLELIICGLEKQLKCESGGPADALDDFDLDMLPADALDDFDLDMLPADALDDFDLDMLP
GSMEGRGSLLTCGDVEENPGPGSSELIKENMHMKLYMEGTVDNHHFKCTSEGEGKPYEGTQTM
RIKVVEGGPLPFAFDILATSFLYGSKTFINHTQGIPDFFKQSFPEGFTWERVTTYEDGGVLTATQD
TSLQDGCLIYNVKIRGVNFTSNGPVMQKKTLGWEAFTETLYPADGGLEGRNDMALKLVGGSHL
IANIKTTYRSKKPAKNLKMPGVYYVDYRLERIKEANNETYVEQHEVAVARYCDLPSKLGHKLN*

### Notch NRR (Negative Regulatory Region)—Binding Antibodies

Notch is typically activated by ligands expressed on adjacent cells, but inhibited when ligands are expressed on the same cell through a mechanism known as “cis-inhibition”. This cis-interaction serves to prevent cells from receiving signals from their neighbors, and also prevents spontaneous “ligand independent” background activation, reducing Notch background activity. As demonstrated herein, membrane-tethered anti-NRR scFvs can be used as genetically encoded Notch inhibitors. These scFvs are derived from antibodies that bind and stabilize the NRR region of Notch receptors, preventing their activation. As shown herein, these synthetic inhibitor proteins can be used to regulate both endogenous Notch and synthetic Notch (“SynNotch”) activity in a manner similar to ligand cis-inhibition.

As known by those of skill in the art, “single-chain Fv” or “scFv” antibody fragments comprise the VH and VL domains of an antibody as a single polypeptide chain. Generally, the Fv polypeptide further comprises a polypeptide linker between the VH and VL domains, which enables the scFv to form the desired structure for antigen binding. For a review of scFv, see Pluckthun in The Pharmacology of Monoclonal Antibodies, Vol 113, Rosenburg and Moore eds. Springer-Verlag, New York, pp. 269-315 (1994).

In one embodiment, binding of the anti-Notch NRR-binding antibody inhibits ligand-independent activation of regulated intramembrane proteolysis resulting to Notch signaling, SynNotch signaling, or at least another signaling outcome when bound to the mutant Notch NRR.

In one embodiment, the anti-Notch NRR-binding antibody is a NRR-binding agonist capable of activating Notch receptors upon cleavage and separation from the fused mutant NRR.

In one embodiment, the NRR-binding antibody is are a Notch NRR-binding scFv, Notch NRR-binding nanobody, or Notch NRR-binding scFab.

In one embodiment, the NRR-binding antibody is are a Notch NRR-binding scFv. In one embodiment, the Notch NRR-binding scFv comprises, from N-terminal to C-terminal and in covalent linkage, a V_{H} domain, a linker domain, and a V_{L} domain.

In one embodiment, the scFv is selected from any one of SEQ ID NOs: 7-12

In one embodiment, the Notch NRR-binding scFv binds to NRR1, NRR2, NRR3.

In one embodiment, the Notch NRR-binding scFv is an anti-NRR1 scFv. In one embodiment, the NRR1 comprises a sequence of SEQ ID NO: 3 or 4.

In one embodiment, the Notch NRR-binding scFv is an anti-NRR2 scFv. In one embodiment, the NRR2 comprises a sequence of SEQ ID NO: 5.

In one embodiment, the Notch NRR-binding scFv is an anti-NRR3 scFv. In one embodiment, the NRR3 comprises a sequence of SEQ ID NO: 6.

In one embodiment, the Notch NRR-binding scFv is an anti-NRR2 scFv. In one embodiment, the Notch NRR-binding scFv is an anti-NRR3 scFv.

Human NRR1-WT (TMD's bolded and italicized)

Human NRR1-WT (TMD's bolded and italicized)
(SEQ ID NO: 3)
ILDYSFGGGAGRDIPPPLIEEACELPECQEDAGNKVCSLQCNNHACGWD
GGDCSLNFNDPWKNCTQSLQCWKYFSDGHCDSQCNSAGCLFDGFDCQRA
EGQCNPLYDQYCKDHFSDGHCDQGCNSAECEWDGLDCAEHVPERLAAGT
LVVVVLMPPEQLRNSSFHFLRELSRVLHTNVVFKRDAHGQQMIFPYYGR
EEELRKHPIKRAAEGWAAPDALLGQVKASLLPGGSEGGRRRRELDPMDV
RGSIVYLEIDNRQCVQASSQCFQSATDVAAFLGALASLGSLNIPYKIEA
VQSETVEPPPPAQLHFMYVAAAAFVLLFFVGCGVLLSRKRRR
Mouse NRR1-WT (TMD's bolded and italicized)
(SEQ ID NO: 4)
ILDYSFTGGAGRDIPPPQIEEACELPECQVDAGNKVCNLQCNNHACGWD
GGDCSLNFNDPWKNCTQSLQCWKYFSDGHCDSQCNSAGCLFDGFDCQLT
EGQCNPLYDQYCKDHFSDGHCDQGCNSAECEWDGLDCAEHVPERLAAGT
LVLVVLLPPDQLRNNSFHFLRELSHVLHTNVVFKRDAQGQQMIFPYYGH
EEELRKHPIKRSTVGWATSSLLPGTSGGRQRRELDPMDIRGSIVYLEID
NRQCVQSSSQCFQSATDVAAFLGALASLGSLNIPYKIEAVKSEPVEPPL
PSQLHLMYVAAAAFVLLFFVGCGVLLSRKRRR
Mouse NRR2-WT (TMD's bolded and italicized)
(SEQ ID NO: 5)
LYTAPTSTPPATCQSQYCADKARDGICDEACNSHACQWDGGDCSLTMED
PWANCTSTLRCWEYINNQCDEQCNTAECLFDNFECQRNSKTCKYDKYCA
DHFKDNHCDQGCNSEECGWDGLDCASDQPENLAEGTLIIVVLLPPEQLL
QDSRSFLRALGTLLHTNLRIKQDSQGALMVYPYFGEKSAAMKKQKMTRR
SLPEEQEQEQEVIGSKIFLEIDNRQCVQDSDQCFKNTDAAAALLASHAI
QGTLSYPLVSVFSELESPRNAQLLYLLAVAVVIILFFILLGVIMAKRKR
K
Human NRR3-WT(TMD's bolded and italicized)
(SEQ ID NO: 6)
APAAAPEVSEEPRCPRAACQAKRGDQRCDRECNSPGCGWDGGDCSLSVG
DPWRQCEALQCWRLFNNSRCDPACSSPACLYDNFDCHAGGRERTCNPVY
EKYCADHFADGRCDQGCNTEECGWDGLDCASEVPALLARGVLVLTVLLP
PEELLRSSADFLQRLSAILRTSLRFRLDAHGQAMVFPYHRPSPGSEPRA
RRELAPEVIGSVVMLEIDNRLCLQSPENDHCFPDAQSAADYLGALSAVE
RLDFPYPLRDVRGEPLEPPEPSVPLLPLLVAGAVLLLVILVLGVMVARR
KRE
In one embodiment, the Notch NRR-binding scFv has
a sequence selected from SEQ ID NOs 7- 12.
A2 scFv (low k off; exhibited greatest fold-
activation upon cleavage;
sequence originally from Genentech; Wu et al
Nature 2010)
(SEQ ID NO: 7)
EVQLVESGGGLVQPGGSLRLSCAASGFTFSSYWIHWVRQAPGKGLEWVA
RINPPNRSNQYADSVKGRFTISADTSKNTAYLQMNSLRAEDTAVYYCAR
GSGFRWVMDYWGQGTLVTVSSGGSSRSSSSGGGGSGGGGDIQMTQSPSS
LSASVGDRVTITCRASQDVSTAVAWYQQKPGKAPKLLIYSASFLYSGVP
SRFSGSGSGTDFTLTISSLQPEDFATYYCQQFYTTPSTFGQGTKVEIK
A scFv (high k off; sequence originally from
Genentech; Wu et al Nature 2010)
(SEQ ID NO: 8)
EVQLVESGGGLVQPGGSLRLSCAASGFTFSSYWIHWVRQAPGKGLEWVA
RINPSNGSTNYADSVKGRFTISADTSKNTAYLQMNSLRAEDTAVYYCAR
GSGFRWVMDYWGQGTLVTVSSGGSSRSSSSGGGGSGGGGDIQMTQSPSS
LSASVGDRVTITCRASQDVSTAVAWYQQKPGKAPKLLIYSASFLYSGVP
SRFSGSGSGTDFTLTISSLQPEDFATYYCQQSYTTPPTFGQGTKVEIK
A3 scFv (medium k off; sequence originally from
Genentech; Wu et al Nature 2010)
(SEQ ID NO: 9)
EVQLVESGGGLVQPGGSLRLSCAASGFTFSSYWIHWVRQAPGKGLEWVA
RINPANGSTRYADSVKGRFTISADTSKNTAYLQMNSLRAEDTAVYYCAR
GSGFRWVMDYWGQGTLVTVSSGGSSRSSSSGGGGSGGGGDIQMTQSPSS
LSASVGDRVTITCRASQDVSTAVAWYQQKPGKAPKLLIYSASFLYSGVP
SRFSGSGSGTDFTLTISSLQPEDFATYYCQQSFSTPATFGQGTKVEIK
Brontictuzumab scFv (aka OMP-52M51; an antibody
against human NRR1 developed by Oncomed
Pharmaceuticals)
(SEQ ID NO: 10)
QVQLVQSGAEVKKPGASVKISCKVSGYTLRGYWIEWVRQAPGKGLEWIG
QILPGTGRTNYNEKFKGRVTMTADTSTDTAYMELSSLRSEDTAVYYCAR
FDGNYGYYAMDYWGQGTTVTVSSGGSSRSSSSGGGGSGGGGQAVVTQEP
SLTVSPGGTVTLTCRSSTGAVTTSNYANWFQQKPGQAPRTLIGGTNNRA
PGVPARFSGSLLGGKAALTLSGAQPEDEAEYYCALWYSNHWVFGGGTKL
TVL
Anti-NRR2 scFv (sequence originally from
Genentech; Wu et al Nature 2010)
(SEQ ID NO: 11)
EVQLVESGGGLVQPGGSLRLSCAASGYTFSSYGMSWVRQAPGKGLEWVS
YIYPYSGATYYADSVKGRFTISADTSKNTAYLQMNSLRAEDTAVYYCAR
HSGYYRISSAMDVWGQGTLVTVSAGGSSRSSSSGGGGSGGGGDIQMTQS
PSSLSASVGDRVTITCRASQNIKRFLAWYQQKPGKAPKLLIYGASTRES
GVPSRFSGSGSGTDFTLTISSLQPEDFATYYCQQYYRSPHTFGQGTKVE
IKR
Anti-NRR3 scFv
(SEQ ID NO: 12)
EVQLVESGGGLVQPGGSLRLSCAASGYAFTDYWMTWVRQAPGKGLEWVA
EISPNSGGTNFNEKFKGRFTISVDNAKNSLYLQMNSLRAEDTAVYYCAR
GEIRYNWFAYWGQGTLVTVSSGGSSRSSSSGGGGSGGGGDIQMTQSPSS
LSASVGDRVTITCKASQNVGNNIAWYQQKPGKAPKLLIYYASNRYTGVP
SRFSGSGYGTDFTLTISSLQPEDFATYYCQRLYNSPFTFGGGTKVEIK

In those embodiments where amino acid sequence modification(s) of an scFv, such as an scFv of any one of SEQ ID NO: 7-12, is performed to engineer mechanical sensitivity, amino acid sequence variants of the NRR-binding scFv are prepared by introducing appropriate nucleotide changes into the nucleic acid encoding the scFv, or by peptide synthesis. Such modifications include, for example, deletions from, and/or insertions into and/or substitutions of, residues within the amino acid sequences of the scFv or antibody from which it is derived. Any combination of deletion, insertion, and substitution is made to arrive at the final construct, provided that the final construct possesses the desired characteristics. The amino acid changes also can alter post-translational processes of the scFv, such as changing the number or position of glycosylation sites.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions for antibody-based sequences include an antibody with an N-terminal methionyl residue or the antibody fused to a cytotoxic polypeptide. Other insertional variants of an antibody molecule include the fusion to the N- or C-terminus of the antibody to an enzyme (e.g., for ADEPT) or a polypeptide which increases the serum half-life of the antibody.

Another type of variant is an amino acid substitution variant. These variants have at least one amino acid residue in the antibody molecule replaced by a different residue. The sites of greatest interest for substitutional mutagenesis typically are the hypervariable regions of the VH and/or VL domains of the scFv.

Substantial modifications in the biological properties of an scFv can be accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H).

Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class.

Any cysteine residue not involved in maintaining the proper conformation of antibodies or antibody fragments thereof can be substituted, generally with serine, to improve the oxidative stability of the molecule and prevent aberrant crosslinking. Conversely, cysteine bond(s) can be added to the antibody to improve its stability (particularly where the antibody is an antibody fragment such as an Fv fragment).

A particularly preferred type of substitutional variant involves substituting one or more hypervariable region residues of a parent antibody. Generally, the resulting variant(s) selected for further development will have improved biological properties relative to the parent antibody from which they are generated. A convenient way for generating such substitutional variants involves affinity maturation using phage display.

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine can also be used.

Addition of glycosylation sites to the antibodies or antibody fragments thereof described herein is accomplished by altering the amino acid sequence such that it contains one or more of the above-described tripeptide sequences (for N-linked glycosylation sites). The alteration can also be made by the addition of, or substitution by, one or more serine or threonine residues to the sequence of the original antibody (for O-linked glycosylation sites).

Nucleic acid molecules encoding amino acid sequence variants of the scFVs used herein are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

### Mutant NRR

A mutant Notch NRR comprises a mutation that induces ligand-independent activation of regulated intramembrane proteolysis resulting to Notch signaling, SynNotch signaling, or at least another signaling outcome.

Mutating the NRR domain and utilizing an scFv that has high affinity to the mutated NRR (but not the native NRR) in either the cis-clamp or auto-inhibitory receptor configurations allow for a more specific system with reduced off-target effects, such as, e.g., the scFv binding the NRR region on notch receptors of adjacent cells).

In those embodiments of the synthetic or recombinant proteins described herein where one or more of the protein domains is mutated or engineered or modified relative to the endogenous or naturally occurring protein, such as a mutated Notch Negative Regulatory Region (NRR), for such purposes as enhancing binding or efficacy, or stability, techniques known in the art for identifying mutated proteins or domains having one or more desired properties can be used. For example, modified or mutated domains or polypeptides can be produced, for instance, by amino acid substitution, deletion, or addition. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) does not have a major effect on the biological activity of the resulting molecule. Conservative replacements are those that take place within a family of amino acids that are related in their side chains.

Naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Preferred conservative substitutions for use in the synthetic proteins described herein are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu. Whether a change in the amino acid sequence of a synthetic protein results in a functional variant can be readily determined by assessing the desired activity of the variant synthetic protein or polypeptide relative to the non-mutated version of the synthetic protein.

In one embodiment, wherein the mutant Notch NRR comprises at least mutation as compared to a wild-type Notch NRR. Exemplary mutations include point mutations, insertions, deletions, and frame shift mutations.

In one embodiment, wherein the mutant Notch NRR comprises at least point mutation as compared to a wild-type Notch NRR.

In one embodiment, wherein the mutant Notch NRR comprises a V1667D mutation as compared to a wild-type Notch NRR.

In one embodiment, the mutant Notch NRR comprises a mutation disclosed herein in Table 4. Table 4 discloses T-ALL-derived destabilizing human Notch1 NRR mutations along with corresponding positions/mutations in mouse Notch1 NRR.

TABLE 4

| T-ALL NOTCH1 Mutation | Corresponding mutation in mouse Notch1 |
|---|---|
| L1575P | L1574P |
| L1594P | L1593P |
| L1601P | L1600P |
| V1677D | V1666D |
| L1679P | L1668P |
| I1681N | I1670N |
| A1702P | A1691P |

In one embodiment, the mutant Notch NRR has a sequence of SEQ ID NO: 1 or 2.

Human NRR1-V1677D (underlined) (TMD's bolded and
italicized)
(SEQ ID NO: 1)
ILDYSFGGGAGRDIPPPLIEEACELPECQEDAGNKVCSLQCNNHACGWD
GGDCSLNFNDPWKNCTQSLQCWKYFSDGHCDSQCNSAGCLFDGFDCQRA
EGQCNPLYDQYCKDHFSDGHCDQGCNSAECEWDGLDCAEHVPERLAAGT
LVVVVLMPPEQLRNSSFHFLRELSRVLHTNVVFKRDAHGQQMIFPYYGR
EEELRKHPIKRAAEGWAAPDALLGQVKASLLPGGSEGGRRRRELDPMDV
RGSIDYLEIDNRQCVQASSQCFQSATDVAAFLGALASLGSLNIPYKIEA
VQSETVEPPPPAQLHFMYVAAAAFVLLFFVGCGVLLSRKRRR
Mouse NRR1-V1677D (underlined) (TMD's bolded
and italicized)
(SEQ ID NO: 2)
ILDYSFTGGAGRDIPPPQIEEACELPECQVDAGNKVCNLQCNNHACGWD
GGDCSLNFNDPWKNCTQSLQCWKYFSDGHCDSQCNSAGCLFDGFDCQLT
EGQCNPLYDQYCKDHFSDGHCDQGCNSAECEWDGLDCAEHVPERLAAGT
LVLVVLLPPDQLRNNSFHFLRELSHVLHTNVVFKRDAQGQQMIFPYYGH
EEELRKHPIKRSTVGWATSSLLPGTSGGRQRRELDPMDIRGSIDYLEID
NRQCVQSSSQCFQSATDVAAFLGALASLGSLNIPYKIEAVKSEPVEPPL
PSQLHLMYVAAAAFVLLFFVGCGVLLSRKRRR

In one embodiment, the mutant Notch NRR polypeptide comprises a sequence having at least 50%, 60%, 70%, 80%, 90%, 91%, 92%, 93%, 94%, 95%, 96%, 97%, 98%, or 99% sequence identify to SEQ ID NO: 1 or 2.

In one embodiment, the mutant NRR domain comprises a S1 furin cleavage loop.

In one embodiment, the mutant NRR domain lacks a S1 furin cleavage loop.

### Linkers

In various embodiments, the linker is a cleavable linker. A cleavable linker means that the linker can be cleaved to release the two parts the linker is holding together. A cleavable linker can be susceptible to cleavage agents, such as, but not limited to, enzymes, pH, redox potential or the presence of degradative molecules. Examples of such agents: redox agents which are selected for particular substrates or which have no substrate specificity, including, e.g., oxidative or reductive enzymes or reductive agents such as mercaptans, present in cells, that can degrade a redox cleavable linking group by reduction; esterases; amidases; endosomes or agents that can create an acidic environment, e.g., those that result in a pH of 7.1 or lower (see, e.g., the world wide web at www.nature.com/articles/nrc3110) (enzymes that can hydrolyze or degrade an acid cleavable linking group by acting as a general acid, peptidases (which can be substrate specific) and proteases, and phosphatases.

In one embodiment, the cleavable linker is cleaved by protease. Exemplary proteases include, but are not limited to, Matrix Metalloproteinases (MMPs), Urokinase, aka urokinase-type plasminogen activator (uPA), Tobacco Etch Virus (TEV) protease, Enterokinase (enteropeptidase), Prostate-Specific Antigen (PSA), Cathepsins, ADAM-family proteases, ADAM-TSs, Kallikreins, Legumain, Fibroblast activating protein-α (FAP), Renin, Human Rhinovirus (HRV) 3C protease, Bacterial sortases, Thrombin, and Type II Transmembrane Serine Proteases.

Exemplary MMPs include, but are not limited to MMPs-1, -2, -8, -9, -10, -11, -12, -13, -15, -19, -23, -24, -27, -28, and any known MMP.

Exemplary Type II Transmembrane Serine Proteases include, but are not limited to, the human airway trypsin-like protease/differentially expressed in squamous cell carcinoma (HAT/DESC) subfamily, e.g., HAT, DESC1, TMPRSS11A, HAT-like 2, HAT-like 3, HAT-like 4, and HAT-like; the hepsin/TMPRSS subfamily: hepsin, TMPRSS2, TMPRSS3, TMPRSS4, TMPRSS5/spinesin, MSPL (mosaic serine protease large-form), and enteropeptidase; the matriptase subfamily: matriptase-2, matriptase-3, and the unique polyserase-1; and Corn.

As known to those of skill in the art, receptors tend to have three regions or domains: an extracellular domain for binding ligands such as proteins, peptides or small molecules, a transmembrane domain that traverses the cellular membrane, and an intracellular or cytoplasmic domain which frequently can participate in some sort of signal transduction event within a cells, such as phosphorylation. Accordingly, in some embodiments of the aspects described herein, the synthetic proteins can comprise various combinations of extracellular, transmembrane, and intracellular domains derived from naturally occurring domains or engineered versions of such domains.

Exemplary linkers include, but are not limited to, linkers have sequences selected from SEQ ID NO: 20-28.

Prototypical TEV protease substrate aka ‘TEVcs’ sequences (note: various constructs contain different linkers, with or without myc tag (bolded, italicized); minimal TEVcs are underlined.

(SEQ ID NO: 20)
GSNSGGTLEGGGSGENLYFQGGGGGRTGGGSEQKLISEEDLGGGS
(SEQ ID NO: 21)
LEGGGSGENLYFQSGRTGGGSEQKLISEEDLGGGS
(SEQ ID NO: 22
GGGSGENLYFQGGGGS
FLAG-tag (substrate for enterokinase/
enteropeptidase; minimal FLAG peptide is
underlined.
(SEQ ID NO: 23)
GGGSGDYKDDDDKGGGS
Factor-Xa substrate is underlined.
(SEQ ID NO: 24)
GGGSGIEGRGGGGS
MMP2/9 substrate is underlined (with FLAG, bolded
and italicized)
(SEQ ID NO: 25)
LEGGGSGPLGLAGGRTADYKDDDDKGGGSG
MMP2/9-scramble control underlined (control
substrate; not cleaved, bolded and italicized);
(SEQ ID NO: 26)
LEGGGSGLALGPGGRTADYKDDDDKGGGSG
MMP14 substrate underlined (control substrate;
not cleaved, bolded and italicized):
(SEQ ID NO: 27)
LEGGSGGTVRPAHLRDSGGGSGGTRTADYKDDDDKGGGSG
Renin substrate underlined (control substrate;
not cleaved, bolded and italicized):
(SEQ ID NO: 28)
LEGGGSGYIHPFHLVIHNESGGRTADYKDDDDKGGGSG

### Transmembrane Domain

The engineered receptors described herein comprise a transmembrane domain that fused to the NRR-binding antibody. The TM can, e.g., tether the engineered receptor construct in the cell membrane and can be cleaved by a membrane protease, such as γ-secretase. The transmembrane domain can comprise any receptor transmembrane domain (e.g., G-protein coupled receptor (GPCR) domain) that comprises a γ-secretase cleavage site (e.g., a Notch transmembrane domain). In some embodiments, the transmembrane domain is that of a monomeric receptor (e.g., not a tyrosine kinase domain). The engineered receptors described herein rely on processing by γ-secretase, which naturally cleaves and processes type 1 integral membrane proteins, such as Notch, ErbB4, E-cadherin, N-cadherin, ephrin-B2, and CD44. Thus, transmembrane domains derived from Notch, ErbB4, E-cadherin, N-cadherin, ephrin-B2 and Cd44 are specifically contemplated for use with the methods and compositions described herein.

In some embodiments, the transmembrane domain comprises a single γ-secretase cleavage site. In other embodiments, the transmembrane domain comprises 2, 3 or 4 γ-secretase cleavage sites. A γ-secretase cleavage site can comprise a Gly-Val dipeptide sequence, where the enzyme cleaves between the Gly and the Val. For example, in some cases, an S3 ligand-inducible proteolytic cleavage site has the amino acid sequence VGCGVLLS (SEQ ID NO: 13), where cleavage occurs between the “GV” sequence. In some cases, an S3 ligand-inducible proteolytic cleavage site comprises the amino acid sequence GCGVLLS (SEQ ID NO: 14).

In some embodiments, the engineered receptors described herein comprise a transmembrane domain from the Notch receptor and comprises an amino acid sequence that is at least 75%, at least 80%, at least 85%, at least 90%, at least 95%, at least 98%, at least 99%, or 100%, amino acid sequence identity to the following amino acid sequence: HLMYVAAAAFVLLFFVGCGVLL (SEQ ID NO: 15). In such embodiments, the engineered receptor comprises at least one γ-secretase cleavage domain (e.g., at least 2, at least 3 or at least 4 γ-secretase cleavage domains).

In other embodiments, In some cases, the engineered receptors described herein comprise a Notch receptor polypeptide comprising a transmembrane domain and having an amino acid sequence with at least 75%, at least 80%, at least 85%, at least 90%, at least 95%, at least 98%, at least 99%, or 100%, amino acid sequence identity to the following amino acid sequence:

(SEQ ID NO: 16)
IPYKIEAVKSEPVEPPLPSQLHLMYVAAAAFVLLFFVGCGVLLSRKRRR
QLCIQKL;

where the TM domain is underlined; wherein the Notch receptor polypeptide has a length of from 50 amino acids (aa) to 65 aa, e.g., 50, 51, 52, 53, 54, 55, 56, 57, 58, 59, 60, 61, 62, 63, 64, or 65 aa.

In some embodiments, the transmembrane domain or other domains of the engineered receptor polypeptide construct can comprise one or more non-gamma-secretase cleavage sites such as e.g., a metalloproteinase cleavage site, e.g., a cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). For example, the cleavage sequence of MMP-9 is Pro-X-X-Hy (wherein, X represents an arbitrary residue; Hy, a hydrophobic residue), e.g., Pro-X-X-Hy-(Ser/Thr), e.g., Pro-Leu/Gln-Gly-Met-Thr-Ser (SEQ ID NO: 17) or Pro-Leu/Gln-Gly-Met-Thr (SEQ ID NO: 18).

In one embodiment, the transmembrane domain comprises the sequence:

(SEQ ID NO: 19)
PVEPPLPSQLHLMYVAAAAFVLLFFVGCGVLLSRKRRR.

### Engineered Cells

In some cases, a nucleic acid comprising a nucleotide sequence encoding an engineered receptor polypeptide construct as described herein is a recombinant expression vector. In some embodiments, the recombinant expression vector is a viral construct, e.g., a recombinant adeno-associated virus (AAV) construct, a recombinant adenoviral construct, a recombinant lentiviral construct, a recombinant retroviral construct, etc. In some cases, a nucleic acid comprising a nucleotide sequence encoding an engineered receptor polypeptide construct as described herein is a recombinant lentivirus vector. In some cases, a nucleic acid comprising a nucleotide sequence encoding an engineered receptor polypeptide construct as described herein is a recombinant AAV vector.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., Hum Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, the vector is a lentivirus vector. Also suitable are transposon-mediated vectors, such as piggyback and sleeping beauty vectors.

As used herein an “expression vector” refers to a DNA molecule, or a clone of such a molecule, which has been modified through human intervention to contain segments of DNA combined and juxtaposed in a manner that would not otherwise exist in nature. DNA constructs can be engineered to other domains operably linked to nucleic acid segments encoding a desired synthetic or recombinant protein of interest. In addition, an expression vector can comprise additional DNA segments, such as promoters, transcription terminators, enhancers, and other elements. One or more selectable markers can also be included. DNA constructs useful for expressing cloned DNA segments in a variety of prokaryotic and eukaryotic host cells can be prepared from readily available components or purchased from commercial suppliers.

Expression vectors can also comprise DNA segments necessary to direct the secretion of a polypeptide or protein of interest. Such DNA segments can include at least one secretory signal sequence. Secretory signal sequences, also called leader sequences, prepro sequences and/or pre sequences, are amino acid sequences that act to direct the secretion of mature polypeptides or proteins from a cell. Such sequences are characterized by a core of hydrophobic amino acids and are typically (but not exclusively) found at the amino termini of newly synthesized proteins. Very often the secretory peptide is cleaved from the mature protein during secretion. Such secretory peptides contain processing sites that allow cleavage of the secretory peptide from the mature protein as it passes through the secretory pathway. A recombinant protein of interest can contain a secretory signal sequence in its original amino acid sequence, or can be engineered to become a secreted protein by inserting an engineered secretory signal sequence into its original amino acid sequence. The choice of suitable promoters, terminators and secretory signals is well within the level of ordinary skill in the art. Expression of cloned genes in cultured mammalian cells and in E. coli, for example, is discussed in detail in Sambrook et al. (Molecular Cloning: A Laboratory Manual, Fourth Edition, Cold Spring Harbor, N.Y., 2012; which is incorporated herein by reference in its entirety).

Also provided herein are host cells genetically modified with a nucleic acid encoding an engineered receptor polypeptide construct as described herein, i.e., host cells genetically modified with a nucleic acid comprising a nucleotide sequence encoding an engineered receptor polypeptide construct as described herein. Also provided herein are methods of modulating an activity of a cell that expresses an engineered receptor polypeptide construct as described herein. The method generally involves contacting the cell with a ligand that binds the at least one ligand binding site in the extracellular binding domain or placing the cell in an environment where it can bind to a cellular antigen or soluble ligand. Binding of the ligand to the ligand binding site induces cleavage of the engineered receptor polypeptide construct at the one or more γ-secretase cleavage sites, thereby releasing the intracellular domain. Release of the intracellular domain modulates an activity of the cell.

Also provided herein is an engineered cell comprising (i) a nucleic acid sequence encoding a Notch NRR-binding scFv, (ii) a linker, and (iii) a nucleic acid sequence encoding a synthetic Notch receptor protein comprising a mutated Notch NRR, wherein the mutant Notch NRR comprises a mutation that induces ligand-independent activation of regulated intramembrane proteolysis resulting to Notch signaling, SynNotch signaling, or at least another signaling outcome.

In some embodiments, a cell is transfected or transformed with a nucleic acid sequence encoding the synthetic protein of interest.

As used herein, the term “transfection” is used to refer to the uptake of an exogenous nucleic acid by a cell, and a cell has been “transfected” when the exogenous nucleic acid has been introduced inside the cell membrane. A number of transfection techniques are well known in the art and are disclosed herein. See, e.g., Graham et al., 1973, Virology 52:456; Sambrook et al., Molecular Cloning, A Laboratory Manual (Cold Spring Harbor Laboratories, 1989); Davis et al., Basic Methods in Molecular Biology (Elsevier, 1986); and Chu et al., 1981, Gene 13:197. Such techniques can be used to introduce one or more exogenous nucleic acids into suitable host cells.

Suitable techniques of transfection for use with the compositions and methods described herein include, but are not limited to calcium phosphate-mediated transfection, DEAE-dextran mediated transfection, and electroporation. Cationic lipid transfection using commercially available reagents including the Boehringer Mannheim Transfection Reagent (N.fwdarw.1-(2,3-Dioleoyloxy)propyl-N,N,N-trimethyl ammoniummethylsulfate, Boehringer Mannheim, Indianapolis, Ind.) or LIPOFECTIN or LIPOFECTAMIN or DMRIE reagent (GIBCO-BRL, Gaithersburg, Md.) can also be used.

The term “transformation” as used herein refers to a change in a cell's genetic characteristics, and a cell has been transformed when it has been modified to contain a new DNA. For example, a cell is transformed where it is genetically modified from its native state. Following transfection, the transforming nucleic acid can recombine with that of the cell by physically integrating into a chromosome of the cell, can be maintained transiently as an episomal element without being replicated, or can replicate independently as a plasmid. A cell is considered to have been stably transformed when the transforming nucleic acid is replicated with the division of the cell.

After transfection, the host cell can be maintained either transiently transformed or stably transformed with said nucleic acid or expression vector. Introduction of multiple nucleic acids or xpression vectors, and selection of cells containing the multiple nucleic acids or expression vectors can be done either simultaneously or, more preferably, sequentially. The technique of establishing a cell line stably transformed with a genetic material or expression vector is well known in the art (Current Protocols in Molecular Biology). In general, after transfection, the growth medium will select for cells containing the nucleic acid construct by, for example, drug selection or deficiency in an essential nutrient, which is complemented by a selectable marker on the nucleic acid construct or co-transfected with the nucleic acid construct. Cultured mammalian cells are generally cultured in commercially available serum-containing or serum-free medium. Selection of a medium appropriate for the particular host cell used is within the level of ordinary skill in the art.

Suitable selectable markers for drug selection used with the compositions and methods described herein include, but are not limited to, neomycin (G418), hygromycin, puromycin, zeocin, colchine, methotrexate, and methionine sulfoximine.

A cell to be engineered with synthetic proteins or combinations thereof described herein can be any cell or host cell. As defined herein, a “cell” or “cellular system” is the basic structural and functional unit of all known independently living organisms. It is the smallest unit of life that is classified as a living thing, and is often called the building block of life. Some organisms, such as most bacteria, are unicellular (consist of a single cell). Other organisms, such as humans, are multicellular. A “natural cell,” as defined herein, refers to any prokaryotic or eukaryotic cell found naturally. A “prokaryotic cell” can comprise a cell envelope and a cytoplasmic region that contains the cell genome (DNA) and ribosomes and various sorts of inclusions.

In some embodiments, the cell is a eukaryotic cell. A eukaryotic cell comprises membrane-bound compartments in which specific metabolic activities take place, such as a nucleus. In other embodiments, the cell or cellular system is an artificial or synthetic cell. As defined herein, an “artificial cell” or a “synthetic cell” is a minimal cell formed from artificial parts that can do many things a natural cell can do, such as transcribe and translate proteins and generate ATP.

In some embodiments, the cell is a mammalian cell, an amphibian cell, a reptile cell, an avian cell, or a plant cell.

In some embodiments, the cell is a mammalian cell. In some embodiments, the cell is a human cell. In some embodiments, the cell is a mouse cell. In some embodiments, the cell is rat cell. In some embodiments, the cell is non-human primate cell. In some embodiments, the cell is lagomorph cell. In some cases, the cell is an ungulate cell.

In some embodiments, the cell is an immune cell, e.g., a T cell, a B cell, a macrophage, a dendritic cell, a natural killer cell, a monocyte, etc. In some embodiments, the cell is a T cell. In some embodiments, the cell is a cytotoxic T cell (e.g., a CD8+ T cell). In some embodiments, the cell is a helper T cell (e.g., a CD4+ T cell). In some embodiments, the cell is a regulatory T cell (“Treg”). In some embodiments, the cell is a B cell. In some embodiments, the cell is a macrophage. In some embodiments, the cell is a dendritic cell. In some embodiments, the cell is a peripheral blood mononuclear cell. In some embodiments, the cell is a monocyte. In some embodiments, the cell is a natural killer (NK) cell. In some embodiments, the cell is a CD4+, FOXP3+ Treg cell. In some embodiments, the cell is a CD4+, FOXP3− Treg cell.

In some instances, the cell is obtained from an individual (e.g., autologous or allogeneic to a subject to be treated). For example, in some embodiments, the cell is a primary cell. As another example, the cell is a stem cell or progenitor cell obtained from an individual.

As one non-limiting example, in some embodiments, the cell is an immune cell obtained from an individual. As an example, the cell can be a T lymphocyte obtained from an individual. As another example, the cell is a cytotoxic cell (e.g., a cytotoxic T cell) obtained from an individual. As another example, the cell can be a helper T cell obtained from an individual. As another example, the cell can be a regulatory T cell obtained from an individual. As another example, the cell can be an NK cell obtained from an individual. As another example, the cell can be a macrophage obtained from an individual. As another example, the cell can be a dendritic cell obtained from an individual. As another example, the cell can be a B cell obtained from an individual. As another example, the cell can be a peripheral blood mononuclear cell obtained from an individual.

In some embodiments, the host cell is a somatic cell, e.g. a fibroblast, a hematopoietic cell, a neuron, a pancreatic cell, a muscle cell, a bone cell, a hepatocyte, a pancreatic cell, an epithelial cell, an endothelial cell, a cardiomyocyte, a T cell, a B cell, an osteocyte, and the like.

Provided herein are methods that can be used to modulate an activity of any eukaryotic cell. In some embodiments, the cell is in vivo. In some embodiments, the cell is ex vivo. In some embodiments, the cell is in vitro. Suitable cells include retinal cells (e.g., Müller cells, ganglion cells, amacrine cells, horizontal cells, bipolar cells, and photoreceptor cells including rods and cones, Müller glial cells, and retinal pigmented epithelium); neural cells (e.g., cells of the thalamus, sensory cortex, zona incerta (ZI), ventral tegmental area (VTA), prefrontal cortex (PFC), nucleus accumbens (NAc), amygdala (BLA), substantia nigra, ventral pallidum, globus pallidus, dorsal striatum, ventral striatum, subthalamic nucleus, hippocampus, dentate gyrus, cingulate gyrus, entorhinal cortex, olfactory cortex, primary motor cortex, or cerebellum); liver cells; kidney cells; immune cells; cardiac cells; skeletal muscle cells; smooth muscle cells; lung cells; and the like.

Exemplary cells include a stem cell (e.g. an embryonic stem (ES) cell, an induced pluripotent stem (iPS) cell; a germ cell (e.g., an oocyte, a sperm, an oogonia, a spermatogonia, etc.); a somatic cell, e.g. a fibroblast, an oligodendrocyte, a glial cell, a hematopoietic cell, a neuron, a muscle cell, a bone cell, a hepatocyte, a pancreatic cell, etc.

Additional exemplary cells include human embryonic stem cells, fetal cardiomyocytes, myofibroblasts, mesenchymal stem cells, autotransplanted expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

In some embodiments, the cell is an immune cell, a neuron, an epithelial cell, and endothelial cell, or a stem cell. In some embodiments, the immune cell is a T cell, a B cell, a monocyte, a natural killer cell, a dendritic cell, or a macrophage. In some embodiments, the immune cell is a cytotoxic T cell. In some embodiments, the immune cell is a helper T cell. In some embodiments, the immune cell is a regulatory T cell (Treg).

In some embodiments, the cell is a stem cell. In some embodiments, the cell is an induced pluripotent stem cell. In some embodiments, the cell is a mesenchymal stem cell. In some embodiments, the cell is a hematopoietic stem cell. In some embodiments, the cell is an adult stem cell.

Suitable cells include bronchioalveolar stem cells (BASCs), bulge epithelial stem cells (bESCs), corneal epithelial stem cells (CESCs), cardiac stem cells (CSCs), epidermal neural crest stem cells (eNCSCs), embryonic stem cells (ESCs), endothelial progenitor cells (EPCs), hepatic oval cells (HOCs), hematopoietic stem cells (HSCs), keratinocyte stem cells (KSCs), mesenchymal stem cells (MSCs), neuronal stem cells (NSCs), pancreatic stem cells (PSCs), retinal stem cells (RSCs), and skin-derived precursors (SKPs)

In some embodiments, the stem cell is a hematopoietic stem cell (HSC), and the transcription factor induces differentiation of the HSC to differentiate into a red blood cell, a platelet, a lymphocyte, a monocyte, a neutrophil, a basophil, or an eosinophil. In some embodiments, the stem cell is a mesenchymal stem cell (MSC), and the transcription factor induces differentiation of the MSC into a connective tissue cell such as a cell of the bone, cartilage, smooth muscle, tendon, ligament, stroma, marrow, dermis, or fat.

In some embodiments, the cell is genetically modified to express two different engineered receptor constructs as described herein or alternatively, an engineered receptor construct as described herein in combination with a second expression construct (e.g., a chimeric antigen receptor expression construct).

In some embodiments, the cell is genetically modified to express an engineered receptor polypeptide construct as described herein; and is further genetically modified to express a chimeric antigen receptor (CAR). For example, in some embodiments, the host cell is genetically modified with a nucleic acid comprising a nucleotide sequence encoding a CAR, and the intracellular domain of the chimeric polypeptide is a transcriptional activator. In some embodiments, the nucleotide sequence encoding the CAR is operably linked to a transcriptional control element that is activated by the intracellular domain of the chimeric polypeptide. Many CAR polypeptides have been described in the art, any of which is suitable for use herein.

In some embodiments, the CAR comprises an extracellular domain, a transmembrane region and an intracellular signaling domain; where the extracellular domain comprises a ligand or a receptor linked to an optional support region capable of tethering the extracellular domain to a cell surface, and the intracellular signaling domain comprises the signaling domain from the zeta chain of the human CD3 complex (CD3zeta) and one or more costimulatory signaling domains, such as those from CD28, 4-1BB and OX-40. The extracellular domain contains a recognition element (e.g., an antibody or other target-binding scaffold) that enables the CAR to bind a target. In some embodiments, a CAR comprises the antigen binding domains of an antibody (e.g., an scFv) linked to T-cell signaling domains. In some embodiments, when expressed on the surface of a T cell, the CAR can direct T cell activity to those cells expressing a receptor or ligand for which this recognition element is specific. As an example, a CAR that contains an extracellular domain that contains a recognition element specific for a tumor antigen can direct T cell activity to tumor cells that bear the tumor antigen. The intracellular region enables the cell (e.g., a T cell) to receive costimulatory signals. The costimulatory signaling domains can be selected from CD28, 4-1BB, OX-40 or any combination of these. Exemplary CARs comprise a human CD4 transmembrane region, a human IgG4 Fc and a receptor or ligand that is tumor-specific, such as an IL13 or IL3 molecule.

The extracellular domain is made up of a soluble receptor ligand (that is specific for a target tumor antigen or other tumor cell-surface molecule) linked to an optional support region capable of tethering the extracellular domain to a cell surface. In some embodiments, the CAR is a heterodimeric, conditionally active CAR, as described in WO 2014/127261. In some embodiments, the heterodimeric, conditionally active CAR is activated by: i) binding an antigen for which the CAR is specific; and ii) a dimerizing agent that induces dimerization of the two polypeptide chains of the heterodimeric, conditionally active CAR. The dimerizing agent can be a small molecule, or can be light.

Once a drug resistant cell population is established, individual clones may be selected and screened for high expressing clones. Methods of establishing cloned cell line are well known in the art, including, but not limited to, using a cloning cylinder, or by limiting dilution. Expression of the recombinant protein of interest from each clone can be measured by methods such as, but not limited to, immunoassay, enzymatic assay, or chromogenic assay. A cell line stably transformed with a first nucleic acid construct may be then used as host cell for transfection with a second or more nucleic acid constructs, and subjected to different drug selections.

By “cell culture” or “culture” is meant the growth and propagation of cells outside of a multicellular organism or tissue. Suitable culture conditions for mammalian cells are known in the art. See e.g. Animal cell culture: A Practical Approach, D. Rickwood, ed., Oxford University Press, New York (1992). Mammalian cells can be cultured in suspension or while attached to a solid substrate. Fluidized bed bioreactors, hollow fiber bioreactors, roller bottles, shake flasks, or stirred tank bioreactors, with or without microcarriers, can be used.

As used herein, “cell culture medium” is a media suitable for growth of animal cells, such as mammalian cells, in in vitro cell culture. Cell culture media formulations are well known in the art. Typically, cell culture media are comprised of buffers, salts, carbohydrates, amino acids, vitamins and trace essential elements. “Serum-free” applies to a cell culture medium that does not contain animal sera, such as fetal bovine serum. Various tissue culture media, including defined culture media, are commercially available, for example, any one or a combination of the following cell culture media can be used: RPMI-1640 Medium, RPMI-1641 Medium, Dulbecco's Modified Eagle's Medium (DMEM), Minimum Essential Medium Eagle, F-12K Medium, Ham's F12 Medium, Iscove's Modified Dulbecco's Medium, McCoy's 5 Å Medium, Leibovitz's L-15 Medium, and serum-free media such as EX-CELL™ 300 Series (JRH Biosciences, Lenexa, Kans.), among others. Serum-free versions of such culture media are also available. Cell culture media can be supplemented with additional or increased concentrations of components such as amino acids, salts, sugars, vitamins, hormones, growth factors, buffers, antibiotics, lipids, trace elements and the like, depending on the requirements of the cells to be cultured and/or the desired cell culture parameters.

As used herein, the term “comprising” means that other elements can also be present in addition to the defined elements presented. The use of “comprising” indicates inclusion rather than limitation. Accordingly, the terms “comprising” means “including principally, but not necessary solely”. Furthermore, variation of the word “comprising”, such as “comprise” and “comprises”, have correspondingly the same meanings. The term “consisting essentially of” means “including principally, but not necessary solely at least one”, and as such, is intended to mean a “selection of one or more, and in any combination”. Stated another way, the term “consisting essentially of” means that an element can be added, subtracted or substituted without materially affecting the novel characteristics of the invention. This applies equally to steps within a described method as well as compositions and components therein. In other embodiments, the inventions, compositions, methods, and respective components thereof, described herein are intended to be exclusive of any element not deemed an essential element to the component, composition or method (“consisting of”). For example, a composition that comprises elements A and B also encompasses a composition consisting of A, B and C.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, publications, and websites identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

Some embodiments of the technology described herein can be defined according to any of the following numbered paragraphs:
1. A synthetic nucleic acid encoding a mutant Notch NRR (Negative Regulatory Region), wherein the mutant Notch NRR comprises a mutation that induces ligand-independent activation of regulated intramembrane proteolysis resulting to Notch signaling, SynNotch signaling, or at least another signaling outcome.
2. The synthetic nucleic acid of paragraph 1, wherein the mutant Notch NRR comprises at least point mutation as compared to a wild-type Notch NRR.
3. The synthetic nucleic acid of any preceding paragraph, wherein the mutant Notch NRR comprises a V1667D mutation as compared to a wild-type Notch NRR, or wherein the mutant Notch NRR comprises a mutation selected from Table 4.
4. The synthetic nucleic acid of any preceding paragraph, wherein the mutant Notch NRR sequence encodes a protein sequence of SEQ ID NO: 1 or 2.
5. A mutant Notch NRR polypeptide encoded by any preceding paragraph.
6. The mutant Notch NRR polypeptide of any preceding paragraph, wherein the mutant Notch NRR protein comprises a sequence of SEQ ID NO: 1 or 2.
7. A fusion protein comprising:
   i) a Notch NRR (Negative Regulatory Region)-binding antibody, and
   ii) a mutant Notch NRR comprising a mutation that induces ligand-independent activation of regulated intramembrane proteolysis resulting to Notch signaling, SynNotch signaling, or at least another signaling outcome.
8. The fusion protein of any preceding paragraph, wherein the Notch NRR-binding antibody inhibits the ligand-independent activation of Notch signaling by the mutant Notch NRR.
9. The fusion protein of any preceding paragraph, wherein the Notch NRR-binding antibody is a Notch NRR-binding scFv, Notch NRR-binding nanobody, or Notch NRR-binding scFab.
10. The fusion protein of any preceding paragraph, wherein the Notch NRR-binding scFv is an anti-NRR1 scFv.
11. The fusion protein of any preceding paragraph, wherein the NRR1 comprises a of SEQ ID NO: 3 or 4.
12. The fusion protein of any preceding paragraph, wherein the mutant Notch NRR comprises at least point mutation as compared to a wild-type Notch NRR.
13. The fusion protein of any preceding paragraph, wherein the mutant Notch NRR comprises a V1667D mutation as compared to a wild-type Notch NRR.
14. The fusion protein of any preceding paragraph, wherein the mutant Notch NRR protein comprises a sequence of SEQ ID NO: 1 or 2.
15. The fusion protein of any preceding paragraph, wherein the Notch NRR-binding immunoglobulin element is further fused to a transmembrane domain.
16. The fusion protein of any preceding paragraph, wherein the fusion protein further comprises, positioned in between i) and ii), at least one linker.
17. The fusion protein of any preceding paragraph, wherein the Notch NRR-binding immunoglobulin element further comprises at least one linker.
18. The fusion protein of any preceding paragraph, wherein the Notch NRR-binding scFv further comprises at least one linker positioned between the heavy and light chains.
19. The fusion protein of any preceding paragraph, wherein the at least one linker is a cleavable linker.
20. The fusion protein of any preceding paragraph, wherein the cleavable linker is cleaved by a protease, an enzyme, chemical reagent, light, pH, or ultrasound.
21. The fusion protein of any preceding paragraph, wherein the at least one linker is cleaved by a protease selected from the group consisting of Matrix Metalloproteinases (MMPs), Urokinase, aka urokinase-type plasminogen activator (uPA), Tobacco Etch Virus (TEV) protease, Enterokinase (enteropeptidase), Prostate-Specific Antigen (PSA), Cathepsins, ADAM-family proteases, ADAM-TSs, Kallikreins, Legumain, Fibroblast activating protein-α (FAP), Renin, Human Rhinovirus (HRV) 3C protease, Bacterial sortases, Thrombin, and Type II Transmembrane Serine Proteases.
22. The fusion protein of any preceding paragraph, wherein the transmembrane domain comprises the human Notch1 transmembrane domain.
23. The fusion protein of any preceding paragraph, wherein the Notch NRR-binding scFv comprises, from N-terminal to C-terminal and in covalent linkage, a V_{H} domain, a linker domain, and a V_{L} domain.
24. The fusion protein of any preceding paragraph, wherein the scFv is selected from any one of SEQ ID NOs: 7-12.
25. The fusion protein of any preceding paragraph, wherein the fusion protein further comprising a signal sequence at its N-terminal.
26. A fusion protein comprising:
   i) a Notch NRR (Negative Regulatory Region)-binding scFv,
   ii) at lease one linker, and
   iii) a mutant Notch NRR, mutant Notch NRR comprises a mutation that induces ligand-independent activation of regulated intramembrane proteolysis resulting to Notch signaling, SynNotch signaling, or at least another signaling outcome.
27. An nucleic acid sequence encoding any of the fusion proteins of any preceding paragraph.
28. The nucleic acid of any preceding paragraph, wherein the synthetic nucleic acid isolated.
29. A synthetic Notch receptor protein comprising, from N-terminal to C-terminal and in covalent linkage, (i) a scFv that binds to an at least one Notch NRR, (ii) at least one linker, (iii) a mutated Notch NRR, wherein the mutant Notch NRR comprises a mutation that induces ligand-independent activation of regulated intramembrane proteolysis resulting to Notch signaling, SynNotch signaling, or at least another signaling outcome, (iv) a transmembrane domain, and (v) an intracellular domain.
30. The synthetic protein of any preceding paragraph, wherein the mutant Notch NRR comprises a V1667D mutation as compared to a wild-type Notch NRR.
31. The synthetic protein of any preceding paragraph, wherein the mutant Notch NRR has a sequence of SEQ ID NO: 1 or 2.
32. The synthetic protein of any preceding paragraph, wherein the at least one linker is a cleavable linker.
33. The synthetic protein of any preceding paragraph, wherein the cleavable linker is cleaved by a protease, an enzyme, chemical reagent, light, pH or ultrasound.
34. The synthetic protein of any preceding paragraph, wherein the at least one linker is cleaved by a protease selected from the group consisting of: Matrix Metalloproteinases (MMPs), Urokinase, aka urokinase-type plasminogen activator (uPA), Tobacco Etch Virus (TEV) protease, Enterokinase (enteropeptidase), Prostate-Specific Antigen (PSA), Cathepsins, ADAM-family proteases, ADAM-TSs, Kallikreins, Legumain, Fibroblast activating protein-α (FAP), Renin, Human Rhinovirus (HRV) 3C protease, Bacterial sortases, Thrombin, and Type II Transmembrane Serine Proteases
35. The synthetic protein of any preceding paragraph, wherein the transmembrane domain comprises the human Notch1 transmembrane domain.
36. The synthetic protein of any preceding paragraph, wherein the scFv comprises, from N-terminal to C-terminal and in covalent linkage, a V_{H} domain, a linker domain, and a V_{L} domain.
37. The synthetic protein of any preceding paragraph, wherein the scFv is selected from any one of SEQ ID NOs: 7-12.
38. The synthetic protein of any preceding paragraph, further comprising a signal sequence N-terminal to the scFv.
39. An isolated nucleic acid sequence encoding the synthetic protein of any preceding paragraph.
40. An engineered cell comprising the isolated nucleic acid sequence of any preceding paragraph.
41. The engineered cell of any preceding paragraph, wherein the engineered cell is an engineered T cell, an engineered stem cell, an engineered innate immune cell, or an engineered natural killer cell.
42. An engineered cell comprising (i) a nucleic acid sequence encoding a Notch NRR-binding antibody, (ii) at least one linker, and (iii) a nucleic acid sequence encoding a synthetic Notch receptor protein comprising a mutated Notch NRR, wherein the mutant Notch NRR comprises a mutation that induces ligand-independent activation of regulated intramembrane proteolysis resulting to Notch signaling, SynNotch signaling, or at least another signaling outcome.
43. The engineered cell of any preceding paragraph, wherein the engineered cell is an engineered T cell, an engineered stem cell, an engineered innate immune cell, or an engineered natural killer cell.
44. The engineered cell of any preceding paragraph, wherein the at least one linker is a cleavable linker.
45. A protease regulatable Notch signaling modulator, the modulator comprising an anti-NRR antibody fused to a mutant NRR, having at least one protease cleavable linker positioned between the anti-NRR immunoglobulin element and mutant NRR.
46. The protease regulatable Notch signaling modulator of any preceding paragraph, wherein the mutant NRR is an NRR from Notch1, Notch2, or Notch3.
47. The protease regulatable Notch signaling modulator of any preceding paragraph, wherein the anti-NRR antibody is an anti-NRR scFv.
48. The protease regulatable Notch signaling modulator of any preceding paragraph, wherein the anti-NRR scFv binds to NRR1, NRR2, NRR3.
49. The protease regulatable Notch signaling modulator of any preceding paragraph, wherein the at least one protease cleavable linker is recognized by TEV, an MMP, other cancer-associated proteolytic enzymes, a chemical reagent, light, acidic pH or ultrasound.
50. The protease regulatable Notch signaling modulator of any preceding paragraph, further comprising at least one AND-gate proteins.
51. The protease regulatable Notch signaling modulator of any preceding paragraph, wherein the at least one AND-gate protein provides independent and/or distinct cleavage sites required to make the antigen binding site of the anti-NRR antibody available.
52. The protease regulatable Notch signaling modulator of any preceding paragraph, wherein the mutant NRR domain comprises a S1 furin cleavage loop.
53. The protease regulatable Notch signaling modulator of any preceding paragraph, wherein the mutant NRR domain lacks a S1 furin cleavage loop.
54. The protease regulatable Notch signaling modulator of any preceding paragraph, wherein the anti-NRR immunoglobulin element is a NRR-binding agonist capable of activating Notch receptors upon cleavage and separation from the fused mutant NRR.
55. The protease regulatable Notch signaling modulator of any preceding paragraph, wherein modulation is an increase in Notch signaling. The protease regulatable Notch signaling modulator of any preceding paragraph, wherein modulation is a decrease in Notch signaling.
56. A protease regulatable Notch signaling modulator, the inhibitor comprising an anti-NRR antibody fused to a transmembrane domain, and a mutant NRR, having at least one protease cleavable linker positioned between the anti-NRR immunoglobulin element fused to the transmembrane domain and mutant NRR.
57. The protease regulatable Notch signaling modulator of any preceding paragraph, wherein cleavage of the at least one protease cleavable linker result in dissociation of the mutant NRR and the anti-NRR immunoglobulin element fused to a transmembrane domain.
58. The protease regulatable Notch signaling modulator of any preceding paragraph, wherein dissociation results in inhibition of Notch signaling via binding of the anti-NRR antibody fused to a transmembrane domain to a receptor.
59. The protease regulatable Notch signaling modulator of any preceding paragraph, wherein modulation is an increase in Notch signaling.
60. The protease regulatable Notch signaling modulator of any preceding paragraph, wherein modulation is a decrease in Notch signaling.
61. A gate protein-synthetic Notch receptor comprising:
   a. a signal sequence;
   b. a ligand binding domain (such as a tumor antigen);
   c. a NRR domain;
   d. a cleavable linker;
   e. a NRR-binding scFv;
   f. a Notch1 TMD;
   g. an intracellular domain
62. The gate protein-synthetic Notch receptor of any preceding paragraph, wherein the ICD is an ICD derived from Gal4, TetR, zinc fingers, Cas9, Cre recombinase, the Notch ICD (NICD), a dominant negative MAML (dnMAML), an inhibitor of transcriptional activity, and a protein with a nucleus-specific activity.
63. The protease regulatable Notch signaling modulator of any preceding paragraph, the modulator is further conjugated to a drug, forming an antibody-drug conjugate.
64. The protease regulatable Notch signaling modulator of any preceding paragraph, wherein the modulator is used to deliver cellular cargo.

### EXAMPLES

### Example 1

### Background

Proteolysis is a critical post-translational modification that can either activate or disable protein function. In addition to its essential intracellular functions, such as maintaining cell homeostasis by degrading misfolded proteins, secreted proteases also play key roles in regulating extracellular processes such as blood clotting, matrix degradation and zymogen activation. Proteolysis is also commonly harnessed in cell signaling, where inactive signaling components are pre-assembled, and their activation can lead to rapid signal amplification. Dysregulated proteolysis has been linked to a wide range of human diseases, and proteases expressed by pathogens play a role in their ability to infect and replicate within hosts. As a result, proteolytic enzymes are extensively investigated as targets in drug development.

The functional repertoire of proteolytic enzymes varies from nonselective digestion to highly regulated limited proteolysis. Due to their fundamental roles in a plethora of processes, proteases constitute one of the largest and most diverse groups of enzymes, with over 600 known human proteases, comprising approximately 2% of the protein-encoding genome¹⁴⁸. The desirable signaling features of proteolytic pathways have led to the development of several synthetic proteolysis-based systems to report and control cell function¹⁴⁹. Recently, these synthetic tools have been expanded to control protein secretion and function by developing protease systems in the secretory pathway¹⁵⁰⁻¹⁵³.

Because of their biological importance, protease activity is normally tightly regulated by the integration of complex signaling pathways. Protease dysregulation leads to many diseases including cardiovascular, inflammation, cancer, osteoporosis, and neurological disorders. Some of these proteases have been successfully targeted for treating these conditions—generally accomplished with small molecule drugs inhibitors that block a protease's active site. These include angiotensin-converting enzyme (ACE) inhibitors for cardiovascular diseases and NS3 inhibitors for treating Hepatitis C Virus.

Another therapeutic strategy that harnesses proteolysis are protease-activated prodrugs, where a probe or therapeutic moiety is attached to a protease-sensitive domain that makes the conjugate inactive until the target protease cleaves the prodrug, releasing the free drug or probe for imaging¹⁵⁴. This concept has already been successfully implemented to biologics, most notably antibodies. For example, antibody-drug and -peptide conjugates with protease-sensitive linkers have been developed to deliver therapeutic moieties to cancer cells. Proteolysis can also be used to activate antibody binding by fusing a cleavable domain that significantly attenuates binding to the target until activated by protease(s). This can be accomplished using a mimotope or non-binding masking moieties¹⁵⁵⁻¹⁵⁷. Furthermore, these protease-activated antibody binding domains have been extended to cell-based medicine applications, as was recently demonstrated in an anti-EGFR pro-antibody CAR receptor¹⁵⁸.

Although immense progress has been made in selectively inhibiting, measuring, or responding to the activity of specific proteases in vivo, there lacks a genetically encodable toolkit to design sense-and-respond behaviors in cells towards extracellular proteolysis. Ideally a fully customizable protease-activated receptor would achieve the following design criteria: programmable sensitivity to a target protease requiring minimal reengineering of the scaffold, conserved activation mechanism permitting it to function in various cell types, and modular ICD for programming designer gene circuits. Herein, the inventors provide data showing their development of modular extracellular protease-activated transcriptional switches. These genetically encodable sensors open new avenues to control pericellular proteolysis in living cells, and enabling proteases to serve as targets for cell-based therapies. Additionally presented herein are protease-activated Notch agonists for potential applications in selectively blocking pathogenic Notch activity in diseased tissues with aberrant proteolysis.

### Design and Generation of a Protease Activated Notch-Based Receptor

Here, inventor use the Notch1 receptor to build a platform to develop a genetically encodable receptor to sense extracellular proteolysis. As Notch is critical for cell-fate determination and survival in many contexts, its dysregulation, gain- or loss-of-function mutations are implicated in many developmental conditions and diseases. For example, T-cell acute lymphoblastic leukemia cancers often contain NOTCH1 mutations that activate in a ligand-independent manner. This has led to extensive biochemical, structural and biophysical characterizations of natural Notch and disease variants. Our molecular design is built upon this body of research.

Normal Notch requires mechanical force for activation. The Negative Regulatory Region (NRR) is the mechanosensitive domain of the receptor that contains the cryptic proteolytic site, known as S2, that is exposed and cleaved by ADAM when the receptor undergoes conformational changes under tension through ligand trans-endocytosis (data not shown). Synthetic Notch (SynNotch) receptors preserve the tension-dependent Notch activation mechanism by retaining the NRR domain, while permitting heterologous substitutions at the LBD and ICD to develop user-defined cell signaling pathways. SynNotch receptors have now been demonstrated in a variety of cell types for in vitro and in vivo applications, suggesting broad utility of the SynNotch platform^{31-35,37-39,159}. These receptors offer a starting point satisfying two of the inventors' design criteria; SynNotch receptors provide versatile cellular output by choosing an ICD of interest to regulate a desired genetic program, and many cell types contain the proteolytic machinery (ADAM and gamma secretase) required to activate Notch-based receptors. Inventors therefore asked whether they could satisfy the remaining criteria—programming specified proteolytic sensitivity—by creating novel NRR domains with distinct autoinhibition to allow for new proteolytic requirements for signaling.

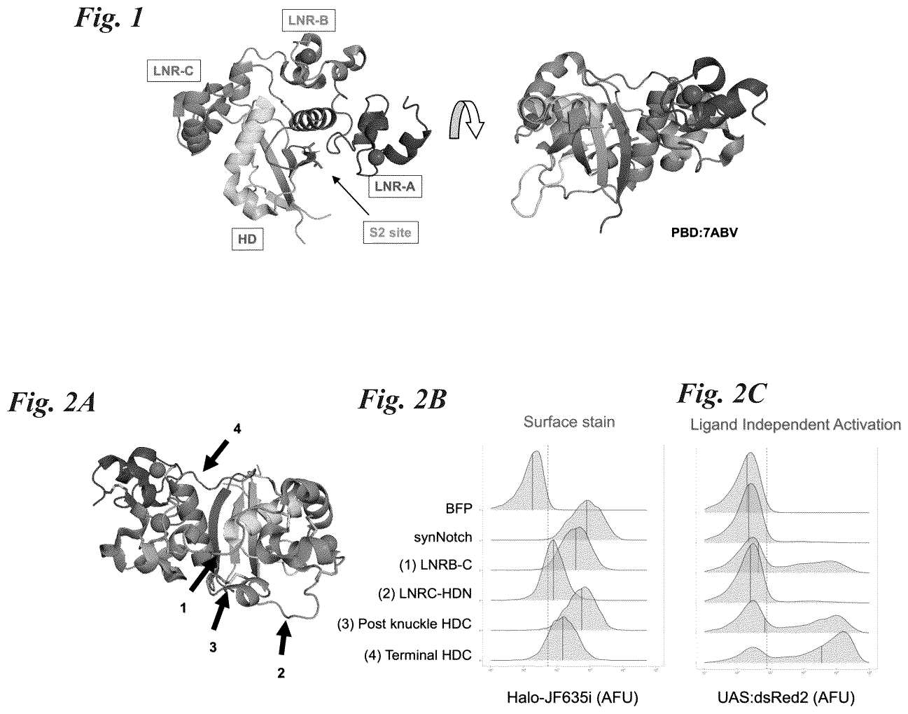

The NRR's autoinhibitory mechanism relies on extensive interdomain interactions from the LNR repeats (LNR-A, -B, and -C) wrapping around the heterodimerization domain (HD) to bury the S2 proteolytic site (FIG. 1)^{6,20}. By evaluating the effect of domain deletions, Gordon et al, 2007 demonstrated that the first two LNR domains (LNR-A and LNR-B) and the interconnecting LNR-AB linker provide the key interactions to protect the S2 site²⁰. The three LNRs and other regions in the periphery of the NRR have been shown to exhibit flexibility in the resting state, suggesting a somewhat dynamic structure for the wild-type domain¹⁶⁰. As an initial hypothesis, the inventors predicted that a synthetic cleavage event within the NRR structure between the LNR and HD domains could lead to a spontaneous dissociation event during natural conformational changes of the NRR that would expose the S2 cleavage site for ADAM proteolysis, resulting in receptor activation. The inventors aimed to identify a permissible site that did not interfere with receptor trafficking and autoinhibition, while allowing receptor activation in response to cleavage by a specific protease. Using a structure-guided approach, the inventors evaluated 4 insertion sites in linker segments between the domains of the NRR and linker segments between HD's secondary structural elements (FIG. 2A, see caption for insertion site details). The inventors inserted a tobacco etch virus substrate (TEVs) which is cleaved by the TEV protease (TEVp). TEV was chosen as the model protease to develop the system described herein since it is a highly specific protease/substrate pair that is commonly used in biotechnology. Additionally, it has recently been used in mammalian cell culture to cleave synthetic extracellular substrates¹⁶¹. Inventors expressed these receptors with a GAL4-VP64 ICD in a U2OS line with an integrated UAS:dsRed2 reporter gene to evaluate their reporter induction activities, and measured receptor surface expression with a cell impermeant Halo-ligand dye (Halo-JF635i). When expressed in these cells inventors observed 3 out of the 4 designs exhibited high basal activation, while the ‘LNRC-HDN’ linker receptor variant exhibited significantly reduced surface expression (FIG. 2). Other tests showed that these receptors did not activate with the addition of TEVp to the media (data not shown). Moving forward, an iterative or a pooled screen approach could be used to improve the LNRC-HDN design by varying the amino acid composition and length of the inserted peptide sequence, which are likely to influence the engineered NRR's folding, autoinhibition, and substrate accessibility. Other insertion sites not tested here could also be evaluated. Although it remains a possibility a protease-activated receptor fulfilling the design criteria could be achieved with these approaches, inventors anticipate that it would require screening several designs to develop a bespoke receptor for each desired protease substrate linker.

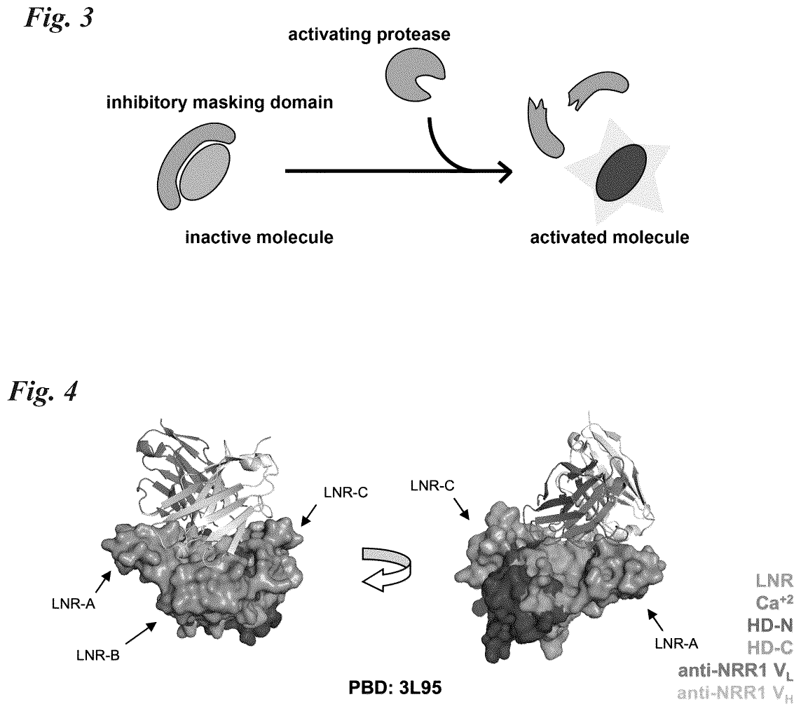

The goal of the experimental design as disclosed herein was to develop a modular design only requiring the substrate linker to be exchanged to direct signaling to a desired protease, similar to the modularity of the SynNotch design for a target antigen by exchanging the LBD. Inventors therefore redirected their efforts in an alternative strategy inspired by zymogen activation, which has spurred the development of protease activated drugs and biologics¹⁵⁴ (FIG. 3). A common strategy in this approach is to fuse an inhibiting domain, commonly referred to as a ‘masking domain’, to an effector domain via a protease sensitive linker. Proteolytic-induced dissociation leads to the effector domain regaining its activity. For example, an antigen mimotope (masking domain) can be used to inhibit an antibody binding its target (effector domain) until the construct is cleaved and mimotope dissociates¹⁶²⁻¹⁶⁵. An advantage of this approach is that the substrate linker can be designed to not contribute to the inhibition of the effector domain, and simply serve as a covalent tether to favor the interaction between the mimotope and effector by proximity. This has enabled researchers to develop constructs which can be sensitive to user-defined proteases by replacing the substrate linker.

### Design of a Protease-Activated Receptor Based on a Cleavable Masking Domain and Hyperactive Notch Mutant

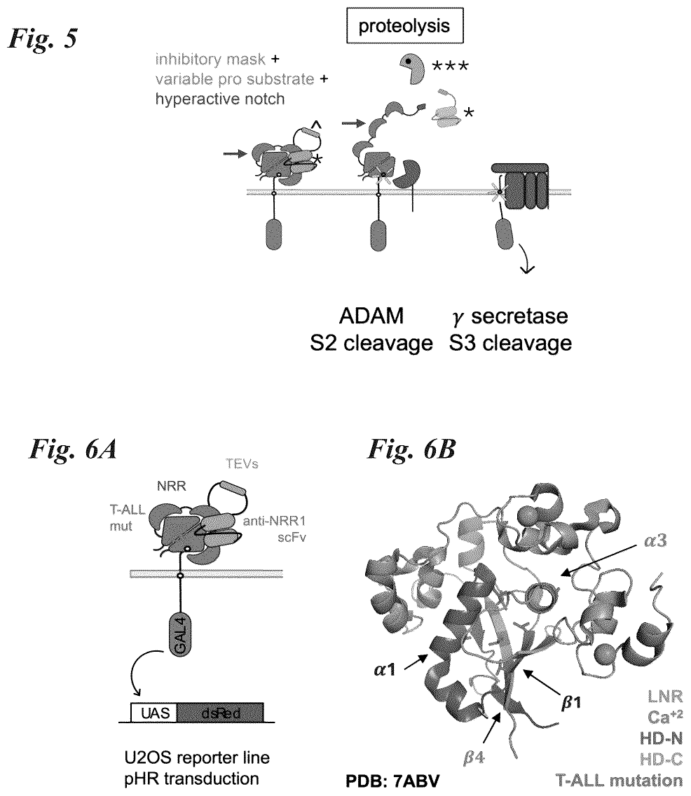

Applying this inhibitory masking domain approach, inventors then developed a modular protease-activated receptor by fusing an inhibitory domain with a protease sensitive linker to a hyperactive Notch (FIGS. 4 and 5). The hyperactive Notch would be required to activate without ligand to render the receptor dependent on proteolysis alone for activation, as opposed to natural Notch's tension requirement. Opportunely, Notch variants with these ligand-independent activities have been reported and shown to form the molecular basis of a class of cancerous gain-of-function NOTCH1 mutations found in over 60% of T-ALL cancers^{23,167}. NOTCH1 signaling plays a critical role at multiple stages of T-cell development. Constitutive forms of Notch show ectopic T-cell development and fail to produce B lymphocytes¹⁶⁸. The most frequent hotspot for mutations in NOTCH1 found in T-ALLs are found within the hydrophobic core of the heterodimerization domain. Most of these are typically single amino acid substitution where nonpolar residues are mutated to polar/charged residues or prolines that disrupt secondary structural elements. These mutations destabilize the NRR in vitro, and are thought to spontaneously undergo a conformational change that exposes the S2 cleavage site independently of ligand-mediated transactivation¹⁶⁹. S2 cleavage is still required in these cancerous forms of the NRR and remains the rate-limiting step for receptor activation.

Targeting these destabilized NOTCH1 NRR has been investigated as a strategy for treating these T-ALL cancers, leading to the development of NRR antagonizing antibodies¹⁷⁰⁻¹⁷³. So far, these antibodies bind mutant and native NRR, therefore inhibiting both ligand-dependent and -independent signaling. Anti-NRR antibodies that antagonize the Notch1 and Notch2 human and mouse paralogs individually are known in the art and were evaluated in both mouse models and human patients for clinical development¹⁷³. The anti-Notch1 NRR antibody (anti-NRR1) bound to NOTCH1 NRR co-crystal structure was reported and indicated that the inhibitory mechanism relies on stabilizing the quiescent NRR's autoinhibited conformation by clamping the LNR-A, LNR-B and HD domains together and interfering with the conformational changes required for receptor activation (FIG. 4)¹⁷³. This NRR autoinhibition stabilization formed the basis of the inventors' prior work in developing the sNRR receptors, in which an scFv of the anti-NRR1 was fused intramolecularly to a SynNotch to increase the tension threshold for receptor activation⁴⁴. Here, inventors investigated whether the anti-NRR1 scFv or a derived variant could serve as the inhibitory mask in the disclosed design. Inventors integrated an anti-NRR1 scFv to impart an inhibitory effect on a hyperactivate Notch variant using characterized T-ALL mutations, with a protease cleavable linker between the two domains. Once cleaved, the inhibitory domain is dissociated and permitted the destabilized NRR to be undergo the conformational changes to expose the S2 site for cleavage by ADAM and the ICD to be released from the plasma membrane through gamma secretase S3 cleavage (FIG. 5).

Characterization of Receptors Containing Anti-NRR1 scFv's and T-ALL Destabilizing NRR Mutations to a Develop a Protease-Activated Receptor Using TEV as a Model Protease.

A major challenge in developing protease-activated biologics is to develop an effective dynamic range—i.e., low activity in the absence and high activity upon proteolysis, respectively. In the inventors application, it was critical for the receptor to remain quiescent in the absence of exogenous protease but induce enough signaling to activate a downstream gene (FIG. 6A). Therefore, a key parameter in the performance of these protease-activated systems is the inhibitory domain's dissociation rate. Too high of a dissociation rate can result in activity in the absence of proteolysis, while a dissociation rate that is too low can have marginal or low activity even if proteolysis cleaves the two molecules' physical association. It was previously demonstrated that the anti-NRR1 scFv can impart mechanical stabilization of SynNotch receptors containing the L1575P or L1594P destabilizing T-ALL mutations, suggesting that the dissociation rate of the inhibitory scFv is low enough to inhibit spontaneous activation when the domains are intramolecularly linked. The ability of the scFv to inhibit these two T-ALL mutations is consistent with the inhibition observed previously with the full length antibody¹⁷³. However whether the scFv would dissociate if the interconnecting linker was cleaved remained an open question. The anti-NRR1 antibody is reported to have nanomolar (nM) affinity to the mouse NRR, which is much higher than the affinity typically found in protease-activated biologics. Taken together, inventors sought to determine whether the anti-NRR1 scFv:T-ALL NRR interaction would be too strong to permit significant dissociation and subsequence receptor activation. To address this possibility, the inventors evaluated variants in our receptor designs from the anti-NRR1 parental antibody which exhibited higher dissociation rates (‘A-3’ and ‘A’ compared to the highest affinity variant ‘A-2’; Table 1)¹⁷⁴.

The inventors next turned to select the destabilizing hyperactive NRR domains from T-ALL mutations. Wu et al. 2010 demonstrated the high affinity anti-NRR1 full-length antibody (‘A-2’) inhibited signaling of the wild-type Notch1 receptor, and 4 T-ALL NOTCH1 mutations: L1575P, L1594P, I1681N, and A1702T¹⁷³. As most T-ALL mutations are found within the hydrophobic core of the HD domain away from the anti-NRR1 binding interface, inventors hypothesize that the anti-NRR1 scFv mask domain variants could potentially bind and inhibit mutations beyond those reported by Wu et al, listed in Table 2 and shown in FIG. 6B¹⁶⁹. Although the affinity of the anti-NRR1 antibody for the mutated NRRs is unknown, inventors have previously seen that fusing them intramolecularly or on separate transmembrane proteins improved SynNotch surface expression, which could benefit our designs^{44,83}.

Interestingly, the effect of the mutations in destabilizing NRR polypeptides in vitro do not strictly correlate with their ligand-independent activities when expressed in cells¹⁶⁹. L1601P, V1677D, and L1679P for example were shown to spontaneously dissociate in native conditions but induce lower signaling compared to less destabilizing mutations that require denaturant for in vitro dissociation¹⁶⁹. This could potentially be attributed to differences in receptor trafficking that could prevent mutated receptors to colocalize with ADAM and gamma secretase for efficient ligand-independent activation. It is known that gamma secretase assembles at post-ER sites, and is therefore not active in the ER¹⁷⁵. Highly destabilizing mutations could misfold in the secretory pathway and be degraded by protein quality control pathways, resulting in lower signaling compared to more stable NRR mutants. Further supporting this notion are the strict quality control mechanisms in the endoplasmic reticulum (ER) to ensure proper folding of the LNR domains. The EroiL thiol oxidase in drosophila is required for proper folding of the LNRs, and its loss of function has been shown to suppress Notch gain of function phenotypes¹⁷⁶. In humans, Notch loss-of-function mutations have also been detected in LNR-A and LNR-B¹⁷⁷. This may be due to quality-control receptor degradation within the ER compartment, thus prevention their transactivation and downstream signaling. Inventors evaluated T-ALL activating mutations that spanned the in vitro NRR dissociation range in combination with the anti-NRR1 scFv variants in hopes of developing a receptor with favorable protease-induced activation characteristics.

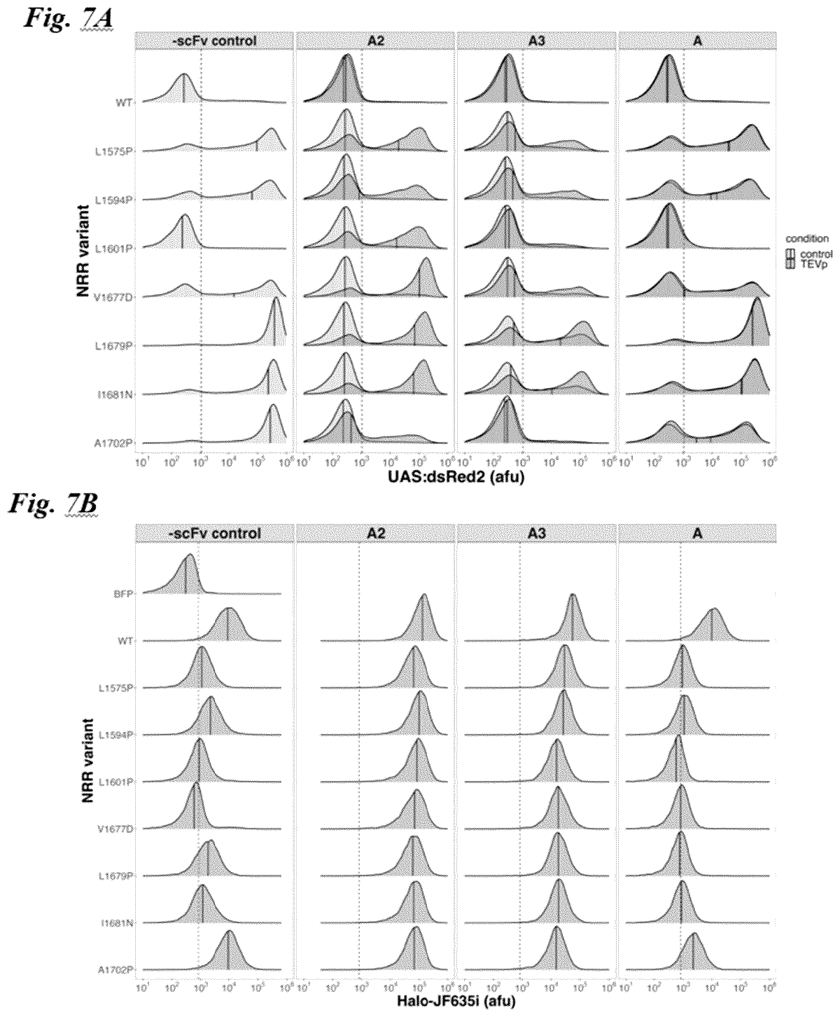

Inventors expressed these receptors in a reporter cell line (FIG. 6A) and evaluated their TEVp-induced activation by analyzing the dsRed2 reporter gene fluorescence and stained for cell surface expression with the cell-impermeant dye Halo-JF635i. Inventors also included receptors containing either no anti-NRR1 scFv or wildtype NRR (WT) as controls. Side-by-side analysis of these receptors showed several successful designs exhibiting low basal activation and high reporter gene activation, all containing the original low dissociation rate (k_{off}) ‘A-2’ variant of the anti-NRR1 scFv (in particular V1677D, L1679P, and 11681N; FIG. 7A). Interestingly, the best performing designs containing the V1677D, L1679P, and 11681N mutations affect hydrophobic residues directed towards the interior of the HD on the same face of the β4 strand (FIG. 6B). This suggests that these designs could share similar binding kinetics with the anti-NRR scFv and protease-induced dissociation conformational changes resulting in efficient proteolytic processing.

Inventors did not observe any protease-induced activation within the designs containing the highest k_{off} anti-NRR variant—‘A’—which is most likely due to its lower affinity leading to spontaneous activation, similar to the no-scFv mask control. Interestingly, the intermediate k_{off} variant ‘A-3’ exhibited lower activation with the addition of TEVp compared to the ‘A-2’ variants. The ‘A-3’ variants exhibit several-fold lower surface expression than the ‘A-2’ variants, suggesting that surface expression could be the key parameter for protease sensitivity differentiating the ‘A-2’ and ‘A-3’ based designs.

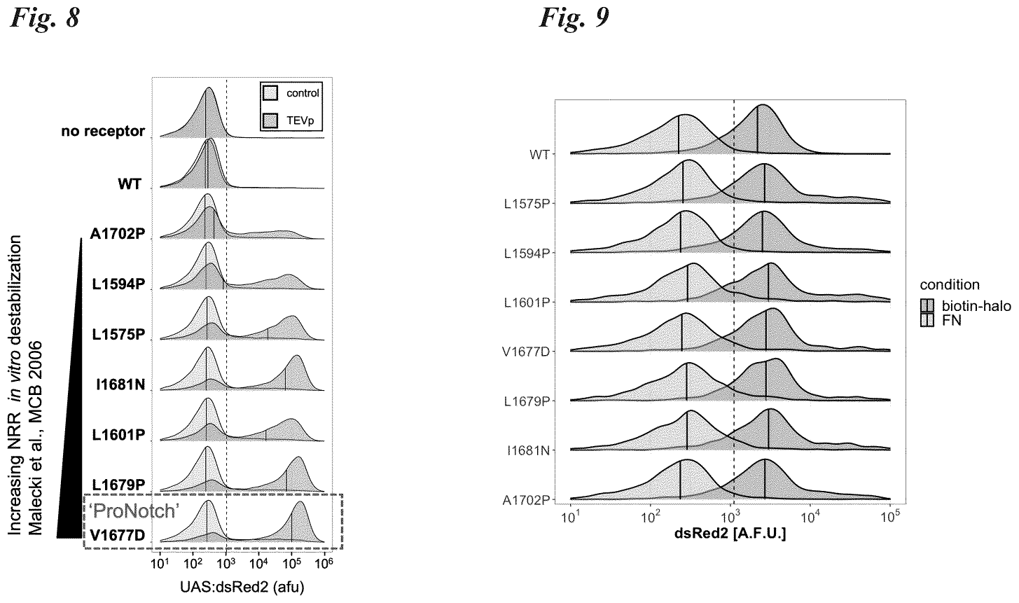

Interestingly, inventors observed all the ‘A-2’ T-ALL variants were surface expressed to a similar extent as the WT control, and appreciably autoinhibited in the absence of exogenous protease. Therefore, the difference in activation could not be explained by receptor accessibility to the extracellular space. Notably, the protease-induced activation of these receptors correlated with the mutation's NRR in vitro destabilization, with L1601P, V1677D and L1679P among the highest activating receptors that result in spontaneous dissociation of NRR polypeptides in vitro¹⁶⁹ (FIG. 8). Interestingly, the no-scFv control for the L1601P NRR does not induce ligand-independent activation, which suggested this mutation's effect on the mouse Notch1 NRR is different than in human NOTCH1 NRR. Further, the ‘A-2’ L1601P receptor is activated by TEVp, indicating that the subcellular localization of the destabilized NRR can influence its activities. The correlation between in vitro destabilization and TEVp-induced signaling however could be due to differences in dissociation kinetics of the anti-NRR scFv for the destabilized NRR's, accessibility of the TEVs substrate, or simply that some of the designs are unable to transduce signaling. Thus, Inventors characterized the ‘A-2’ based receptors further with the aim to better understand their activation behaviors.

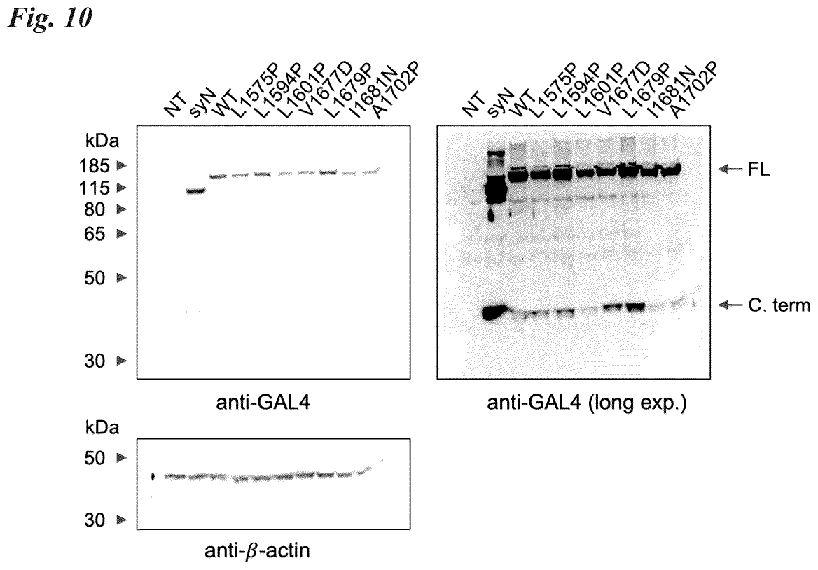

Similar to sNRR, these designs are expected to activate in response to plated ligand if the receptor ECDs undergo sufficient tension. Inventors therefore first evaluated the receptors' activity against plated Halo-ligand to assess their ability to transduce signaling through the natural mode of Notch receptor activation. Inventors observed comparable signaling between the receptors against the non-mutated NRR control, suggesting the designs are all signaling-competent (FIG. 9). Next, inventors investigated the possibility that their designs could have differed S1-processing which may influence signaling. It has been shown that furin cleavage is not obligatory for T-ALL-related NRR mutations to induce ligand-independent signaling. This suggests the conformational changes required for ligand-independent activation can proceed independent of S1 cleavage, and by extension, HD dissociation⁶. Nonetheless, it remained a possibility that S1-resistance could be a factor in the signaling behaviors of the novel receptors disclosed herein. An immunoblot probing for the C-terminal fragment of these receptors (αGAL4) however showed comparable S1 processing between the designs indicating this is unlikely to a significant factor (FIG. 10).

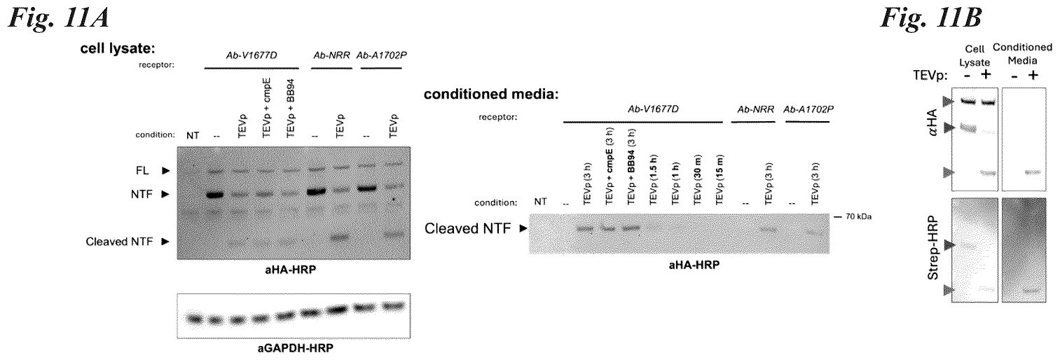

Inventors then evaluated whether these receptors have different TEVs cleavage efficiencies and anti-NRR1 scFv dissociation kinetics. They immunoblotted for the anti-NRR1 scFv mask in cell lysates and conditioned media in cells expressing the non-mutated WT, V1677D, and A1702P NRR receptors in the absence or addition of exogenous TEVp. Inventors than chose to analyze the V1677D and A1702P mutations since they were the designs with the highest and lowest protease-induced reporter activation, respectively. The intensity of the cell-surface receptor specie (the S1-cleaved N-terminal fragment specie; NTF) with the addition of TEVp suggests that the three receptors are cleaved with similar efficiencies (left blot, FIG. 11A). Interestingly, fragments were detected in the conditioned media for all the receptors (right blot, FIG. 11A). This indicates that the anti-NRR1 scFv dissociates from both native and mutated NRR's. Notably, the fraction of the anti-NRR1 scFv found in the conditioned media was observed to be greater for the V1677D receptor compared to the less destabilizing (and lower activating) A 1702P receptor and the non-mutated NRR control. This result indicates the dissociation of the anti-NRR1 scFv may be greater for the V1677D and the other highly destabilized NRR mutants, which aligns with the reporter gene activation observed in FIG. 8. Inventors were able to detect cleaved dissociated scFv fragments within 1 hour of TEVp incubation in this assay. While these results hint at a possible mechanism to explain the differences observed in the reporter gene activation result, further biophysical characterizations was performed to elucidate the manner in which these receptors are activated.

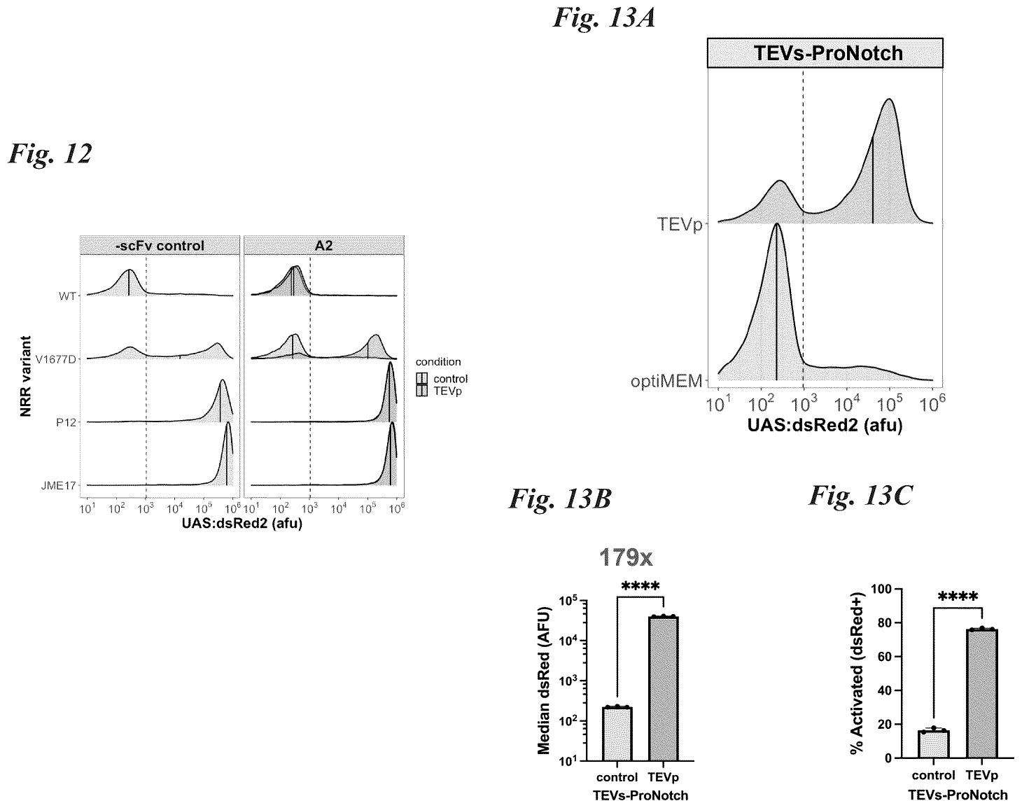

In addition to NRRs bearing point mutations, inventors also tested the ‘P12’ and ‘JME17’ mutations in their designs, which are unique Notch hyperactive mutations consisting of in-frame insertions (14 and 17 amino acids, respectively) that do not affect the stability of the NRR, yet result in strong ligand-independent activation^{169,178}. Both mutations are found at the C-terminal region of the HD domain, and are thought to enhance access to the S2 site to ADAM despite their negligible effect on NRR stability. Inventors evaluated these mutations in their design to test if the anti-NRR1 scFv could autoinhibit these mutated NRRs ligand-independent activities. Inventors found that the anti-NRR1 intramolecular fusion did not appreciably inhibit the spontaneous activation of either mutation (FIG. 12). These results indicate that the ‘P12’ and ‘JME17’ mutated NRRs may not be processed at the canonical S2 or similar site as the other T-ALL mutations evaluated.

For future development and analyzes, inventors chose the ‘A-2’ anti-NRR1 scFv receptor with the V1677D mutation due to its relatively low basal activation and highest protease-induced activation (FIG. 8). The reduced spontaneous activation of the V166D mutation when fused to the higher dissociating anti-NRR1 scFv's (‘A-3’ and ‘A’) compared to the L1679P- and I1681N-based designs further motivated this choice, with the idea that misfolded receptors are less likely to spontaneously activate. This could potential be of concern for when receptors are overexpressed or expressed stably in cells.

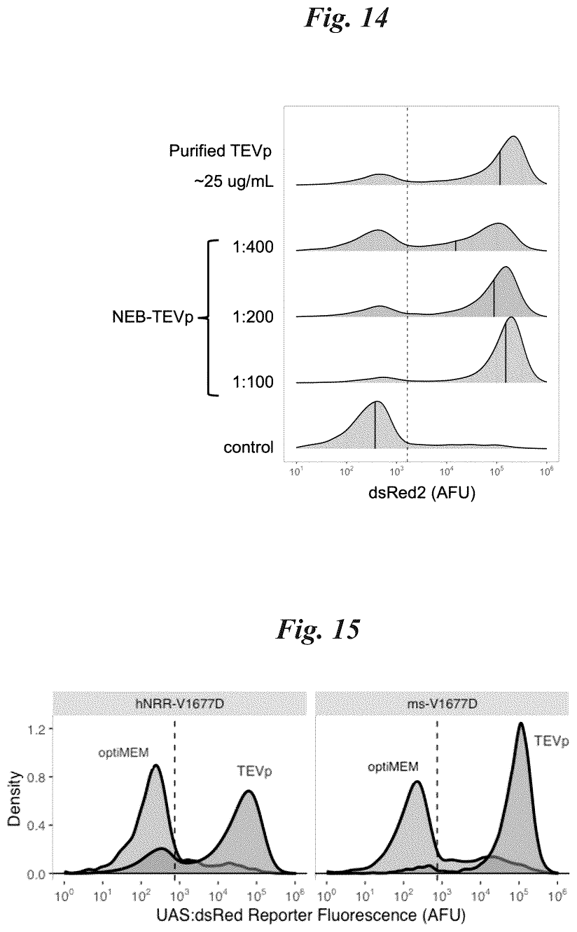

Henceforth, inventors refer to the V1677D receptor and its engineered protease-sensitive derivates as “ProNotch”. The “Pro” designation refers to both intact (pre-cleavage) state as well as its protease sensitivity. Inventors further describe with a prefix the protease substrate linker in the ProNotch receptor design. For example, the TEVs-containing proNotch receptor is referred to as TEVs-ProNotch. Using flow cytometry, inventors quantified the TEVp-induced activation of TEVs-ProNotch in triplicate, observing basal activation (20% dsRed+) and dsRed2 median fold change (179×) comparable to a traditional SynNotch activating on plated ligand (FIG. 13, data not shown). TEVs-ProNotch activation was also shown to be TEVp dose dependent. Furthermore, comparisons using commercially obtained TEVp (NEB, P8112S) and in-house TEVp (recombinantly expressed and purified from E. coli) showed similar activation levels between treated cells (FIG. 14). Note that inventors evaluated purified TEVp to determine the concentration required to activate TEVs-ProNotch, as the NEB-TEVp is sold at enzymatic units. This comparison was also done to ensure that the vendor storage buffer was not confounding any results. Collectively, these results show that ProNotch receptors can exhibit low basal activation and be induced upon treatment with exogenous protease that is dose dependent. Furthermore, the results indicate that both in-house purified and commercial recombinant TEVp can serve as suitable enzyme-based activators for inducing TEVs-ProNotch activity. In summary, inventors used a structure-guided approach with prior biophysical characterizations of the Notch receptor to develop a protease-activated receptor by fusing the anti-NRR1 scFv to a destabilized Notch NRR with a protease sensitive linker.

TABLE 1
Binding kinetics and affinity of anti-
NRR1 antibodies to mouse Notch1 NRR

| | kₒₙ [M⁻¹s⁻¹] | k_{off} [s⁻¹] | K_{d} [M] |
|---|---|---|---|
| A-2 | 1.10 × 10⁵ | 3.40 × 10⁻⁴ | 3.09 × 10⁻⁹ |
| A-3 | 9.20 × 10⁴ | 1.20 × 10⁻³ | 1.30 × 10⁻⁸ |
| A | 9.10 × 10⁴ | 2.10 × 10⁻² | 2.31 × 10⁻⁷ |

Table adapted from Siebel, C. W., and Wu, Y. Anti-Notch1 NRR Antibodies. U.S. Pat. No. 8,846,871 B2, 2014, which is incorporated herein by reference in its entirety.

TABLE 2
T-ALL NRR destabilizing mutations tested in disclosed designs

| T-ALL | Corresponding | | Destabi- | Ligand- |
|---|---|---|---|---|
| NOTCH1 | mutation in | | lization | independent |
| Mutation | mouse Notch1 | Location | in vitro | activities in cells |
| L1575P | L1574P | β1 | +++ | ++* |
| L1594P | L1593P | α1 | ++ | ++ |
| L1601P | L1600P | α1 | +++++ | + |
| V1677D | V1666D | β4 | ++++ | +++ |
| L1679P | L1668P | β4 | +++++ | ++ |
| I1681N | I1670N | β4 | +++ | +++ |
| A1702P | A1691P | α3 | + | ++ |

Table 2 provides a list of the T-ALL NOTCH1 mutations tested in our receptor designs. Relative in vitro destabilization and ligand-independent activities adapted from results in Malecki et al, MCB 2006.

### Analysis of a Humanized ProNotch Receptor

Throughout this work, inventors develop and apply ProNotch with the mouse paralogue of Notch1, similar to SynNotch. A fully-humanized ProNotch receptor could be useful in certain applications where murine antigen immune-reactivity is of concern, such as in cell therapies. As the anti-NRR1 also binds the human NOTCH1 paralogue, inventors evaluated their design with the human NOTCH1 NRR. TEVs-hu-ProNotch was found to activate, albeit at a reduced level compared to the murine TEVs-ProNotch (FIG. 15). This result indicated designs based on human NOTCH1 NRR can be used to develop ProNotch receptors. Furthermore, similar strategies could be used to make systems based on Notch-2, -3, and -4. Structural models for Notch2²⁰, Notch3¹⁷⁹ and inhibitory antibodies^{180,181} would facilitate the development of these new receptors.

### ProNotch is Processed Like Natural Notch Receptors Downstream of Protease Cleavage

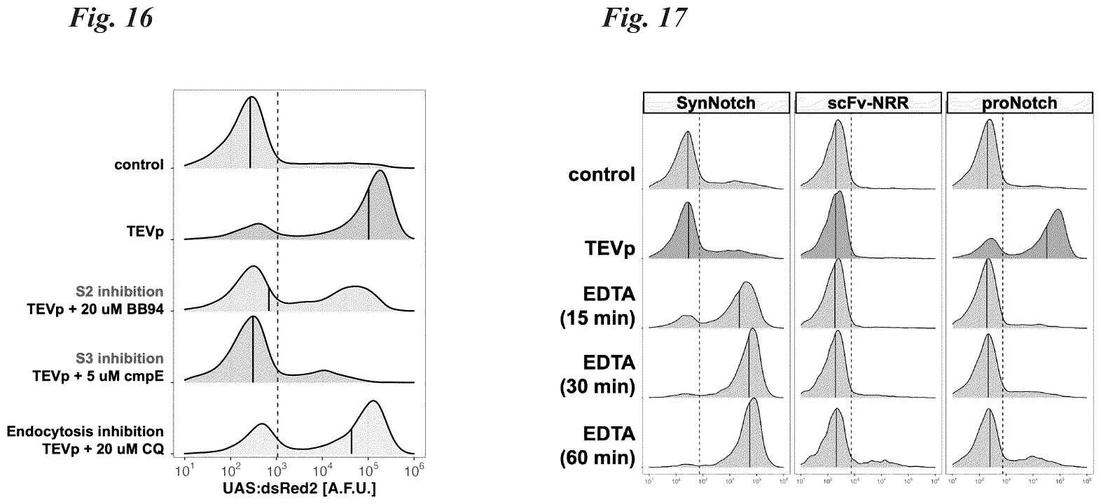

Inventors next evaluated if the ProNotch receptor undergoes similar proteolytic steps as native Notch signaling after the inhibitory mask dissociates-namely S2 cleavage by an ADAM and concomitant cleavage by gamma secretase (FIG. 5). Inventors therefore cotreated TEVs-ProNotch cells with TEVp and Batimastat or compound E, a broad spectrum metalloprotease inhibitor and potent gamma secretase inhibitor, respectively. Inventors first confirmed that these compounds do not inhibit receptor cleavage by TEVp (a serine protease) as expected (FIG. 11). In U2OS reporter cell line expressing TEVs-ProNotch, inventors observed decreases in signaling with each of these compounds, indicating that ProNotch retains a similar proteolytic cascade as natural Notch (FIG. 16). BB94 inhibition was not as complete at compound E. There however remains the possibility that an alternative protease (not inhibited by BB94) may be involved in ProNotch ectodomain shedding. ADAM10 is known to be the canonical metalloprotease for ligand induced Notch signaling, but ADAM17 cleaves Notch in some contexts, such as during overexpression¹⁸². Both ADAM10 and ADAM17 have been shown to cleave T-ALL mutated NRR's, with a greater contribution from ADAM17 observed in C2C12 cells¹⁸². It would be interesting to further characterize the participating sheddase(s) in ProNotch activation, and to determine the subcellular compartment where receptor processing is occurring. To begin to address this, inventors cotreated TEVs-ProNotch cells with TEVp and chloroquine, a lysosomotropism agent that prevents endosomal acidification. Chloroquine thus prevents the maturation and fusion of endosomes and lysosomes. This was to evaluate whether receptor endocytosis and subsequent acidification in the endolysosomal pathway contributes to scFv dissociation, or colocalization with receptor-processing proteases. Chloroquine has been shown to decrease T-ALL cell viability, which is thought to interfere with the intracellular trafficking and processing of destabilized NOTCH1¹⁸³. This may be caused by interference with Notch S3 cleavage, which has been found to occur in an intracellular compartment⁷⁸. When treated with TEVp and chloroquine, inventors observed a slight decrease in activation (FIG. 16). This preliminarily suggets that endocytosis and lysosomal acidification may not be a large contributing factor in ProNotch activation.

The NRR's autoinhibition depends on the presence of calcium ions (FIG. 1), and calcium chelation has been shown ectopically induce ligand-independent activation^{160,184}. Therefore, Notch studies using adhere cells are passaged with non-EDTA dissociative agents to minimize inadvertent Notch activation. Interestingly, sNRR domains were found to provide a dominant autoinhibitory effects that renders sNRR-based receptors resistant to EDTA-induced ligand-independent activation⁴⁴. Even the weakly mechanically stabilizing sNRR_{R103A}, which exhibited similar mechanostability as the original NRR, was resistant to EDTA-induced ligand-independent activation. These results suggested the scFv variants developed as sNRR domains remain bound to the NRR prior to ligand-induced tension. Inventors then evaluated whether our ProNotch receptor retained the EDTA-resistance observed in sNRR. Even at long incubations with EDTA, the ProNotch receptor exhibited similar EDTA-resistance as the non-mutated NRR control with the anti-NRR1 scFv fusion, further supporting that the anti-NRR1 inhibitory mask is bound and inhibits the destabilized NRR prior to proteolysis (FIG. 17).

### Activation of ProNotch in Cells Stably Expressing the Receptor

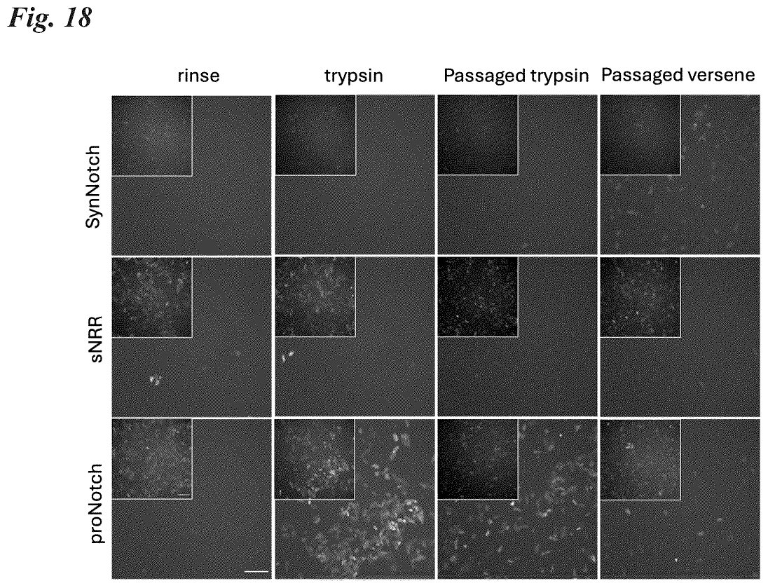

Inventors next evaluated ProNotch in stably expressing cells. They generated stable TEVs-ProNotch-expressing cell populations in two reporter cell lines—the U2OS cell line with an integrated UAS:dsRed2 reporter gene, and the HEK293-FT cell lines with a UAS:H2B-mCherry reporter cell line. When inventors developed stably expressed ProNotch cells, they noticed that routine passaging with 0.25% trypsin (without EDTA) induced signaling that would subside after cell expansion. Inventors had not observed such high levels of ligand-independent activation during routine passaging in our other Notch-based receptor expressing cell lines. An experiment in the U2OS reporter cell line transduced to express either a SynNotch, sNRR, and ProNotch receptor show this phenomenon (FIG. 18). Incubating ProNotch cells with 0.25% trypsin with a shorter incubation time than required to dissociate the U2OS cells from the tissue culture plate resulted in a similar effect, while replacing media did not induce activation (‘trypsin’ and ‘rinse’ conditions). This demonstrated that the activation was not caused by the dissociation from tissue culture plastic and cell replating. Similar induction was observed using the ‘gentler’ recombinant trypsin-like enzyme TrypLE, or with collagenase type IV and dispase II (data not shown). Activation induced by these latter two proteases was a surprise, as they are only intended to cleave ECM components and are used in cell culture applications where it is desired to minimize cleavage of cell surface receptors.

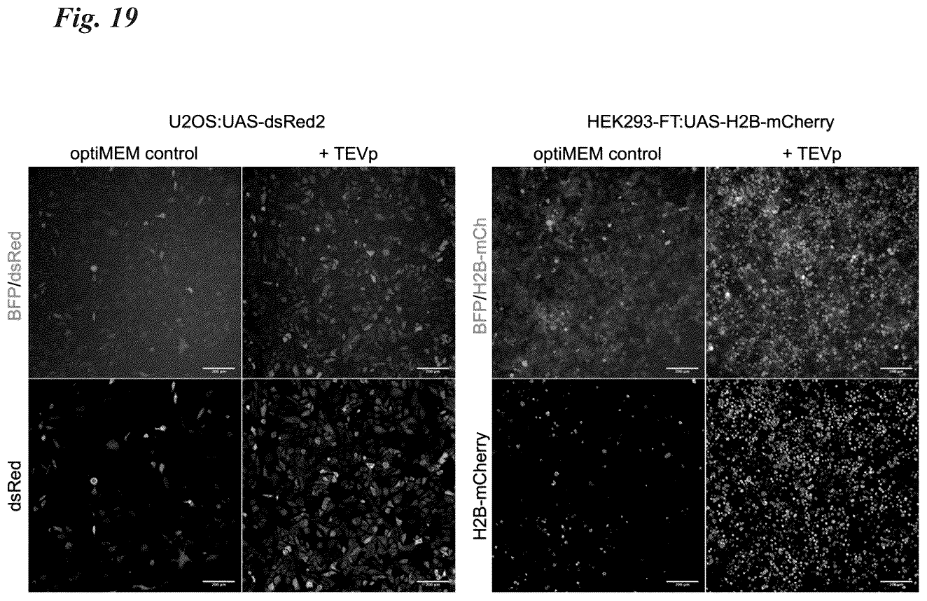

As ProNotch was previously shown to be resistant to EDTA treatment (FIG. 17), inventors additionally evaluated ProNotch signaling activation while passaging receptor-expressing cells with Versene, a non-enzymatic dissociative agent based on EDTA. Versene was found to not induce ligand-independent activation to the same extent as trypsin when passaging U2OS ProNotch cells (FIG. 18). Similar results were observed with ATCC's non-enzymatic dissociative agent or 0.5 mM EDTA in PBS (data not shown). Collectively, inventors find that protease-based dissociative agents induce signaling in ProNotch-expressing cells, which can be inferred to be a result of non-specific ProNotch cleavage. Notch and sNRR domains do not exhibit this activation, suggesting the proteolysis is most likely occurring in the flexible linkers of these receptors such as the anti-NRR1 scFv linker (between V_{H} and V_{L}) or linker between the scFv and NRR. Any cleavage and dissociation of the anti-NRR1 scFv in the ProNotch would result in activation, while cleavage in these regions in SynNotch/sNRR receptors would render them signaling incompetent by dissociating the LBD. To address this issue, inventors found that non-enzymatic dissociative agents such as Versene, ATCC's dissociative reagent, and 0.5 mM EDTA (PBS) can be used to passage ProNotch cells as these receptors are resistant to calcium chelation. The stably expressing U2OS and HEK293-FT TEVs-ProNotch cells were then passaged using versene to prevent inadvertent basal activation, and shown to activate in response to exogenous TEVp (FIG. 19).

In conclusion, inventors found that U2OS and HEK293-FT cells expressing ProNotch require non-enzymatic dissociative reagents to maintain receptor quiescence during routine passaging. For strongly adherent cells such as U2OS, versene incubation and vigorous mechanical dissociation by pipetting is required to dissociate cells from tissue culture plastic. Inventors found that HEK293-FT cells can be dissociated with similar results by pipetting in warm media, as they are weakly adherent to tissue culture plastic. Stably expressing TEVs-ProNotch cells activated in response to exogenous TEVp, but the non-specific activation observed with the various protease-dissociative reagents put into question the protease-specificity of our ProNotch design.

### Protease-Induced Myogenic Conversion of C3H10T1/2 Multipotent Fibroblasts

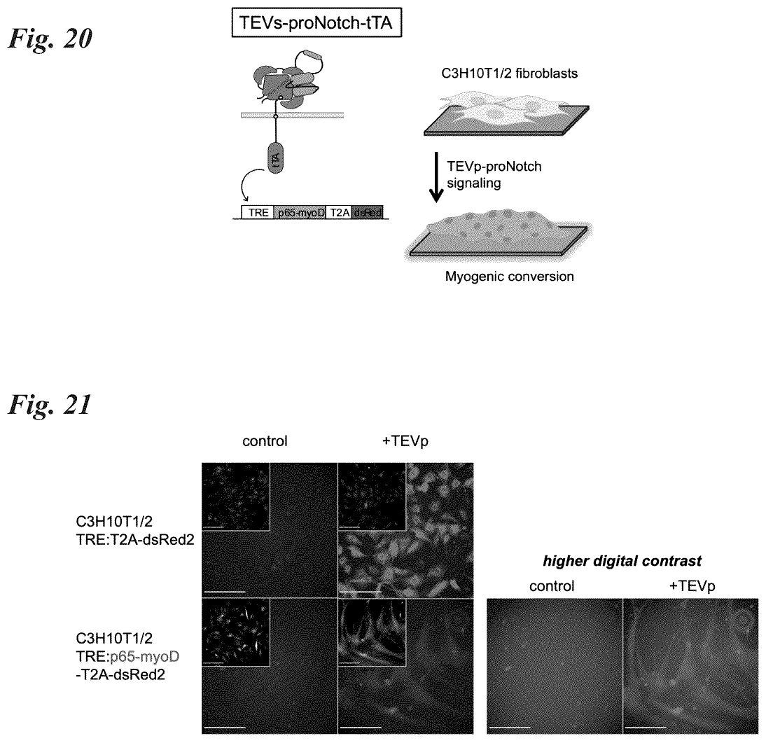

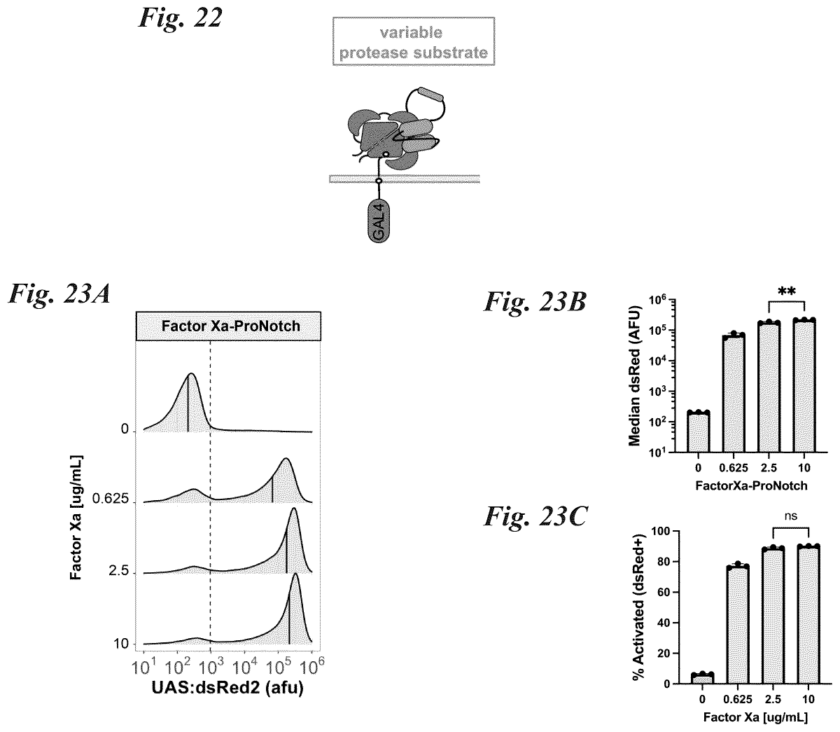

Equipped with a protease-activated receptor, inventors next aimed to specify how a cell responds to proteolytic activity with more complex phenotypic changes than reporter fluorescence expression. In natural systems, cells enact intricate biological outputs based upon proteolysis in their microenvironment. For example, remodeling of the ECM by MMPs is critical to the epithelial-to-mesenchymal transition that occurs to form the 3 germ layers during gastrulation stage of animal development¹⁸⁵. Using a synthetic biology approach, inventors generated mouse embryonic fibroblasts that undergo myogenic differentiation in response to extracellular proteolysis (FIG. 20). To do so, inventors rendered the master regulator of myogenic different, MyoD, dependent on ProNotch activation, analogous to SynNotch activation. TEVs-ProNotch receptors were expressed in these cells such that TEVp-induced signal transduction would induce p65-MyoD expression leading to myogenic conversion. Consistent with the inventors expectations, exogenous TEVp addition resulted in the formation of red fluorescent syncytia (FIG. 21). As a control, inventors induced TEVs-ProNotch signaling in C3H10T1/2 cells containing TRE:T2A -dsRed, lacking the signaling-inducing p65-MyoD. Tests using these cells resulted in red fluorescent cells lacking observable syncitialization, confirming that the observed phenotypes in the experimental condition arose due to TEVp-induced p65-MyoD expression. Collectively, these results indicated that ProNotch function as expected in another cell type, and that the ICD can be exchanged for another transcription factor (tTA for GAL4) to induce a more complex genetic program for a synthetic biology application.

### Orthogonal Activation of Distinct Receptors by Multiple Proteases by Substituting the Protease Substrate Linker

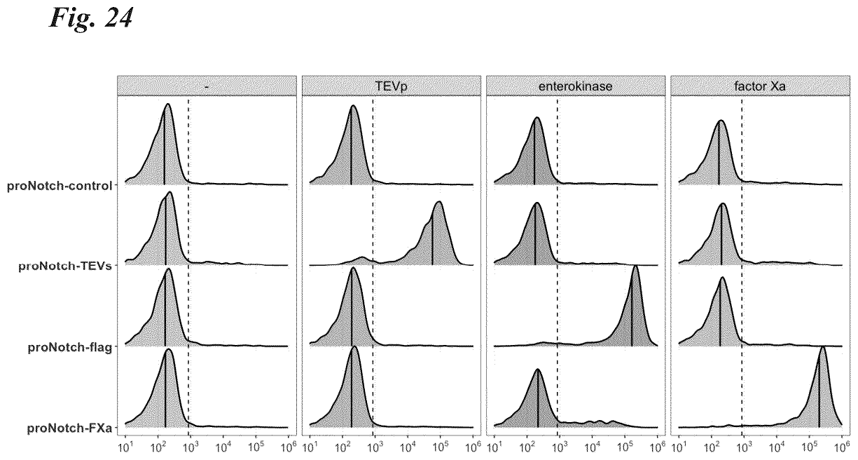

Inventors next evaluated whether the ProNotch design is modular and can be specific for a user-defined protease by exchanging the protease substrate linker (FIG. 23). Inventors first constructed a receptor with a Factor Xa substrate, which was found to be more dose-sensitive to exogenous protease than TEVs-ProNotch with TEVp (FIGS. 14 and 23). Inventors then constructed receptors with flag tag, which is cleaved by enterokinase, and a myc tag linker to serve as a control (protease substrate linkers are listed in Table 3). Inventors preliminary tested enterokinase and Factor Xa since these proteases are not expressed in U2OS cells and can be obtained from vendors. These proteases have less stringent sequence specificity than TEVp, so inventors assessed whether they would observe non-specific activation of ProNotch receptors as was detected with protease-based dissociative agents (FIG. 18). Inventors therefore tested the interaction of each protease (TEVp, enterokinase, Factor Xa) in cells expressing each individual ProNotch. Surprisingly, each receptor was activated by their respective proteases at the high concentrations tested with minimal cross-activation (TEVp; 1:100, enterokinase; 10 μg/mL Factor Xa; 10 μg/mL). These results demonstrated the ProNotch design is modular and can be directed to activate in response to a defined protease (FIG. 24).

TABLE 3
Protease substrate linker sequences used in
Chapter 4 (Table 3 discloses SEQ ID
NOS 41, and 22-26, respectively).

| protease substrate | peptide sequence |
|---|---|
| myc tag control | GGGSEQKLISEEDLGGGS |
| TEVs | GGGSGENLYFQ↓GGGGS |
| enterokinase (flag tag) | GGGSGDYKDDDDK↓GGGS |
| factor Xa | GGGSGIEGR↓GGGGS |
| MMP2/9 | LEGGGSGPLG↓LAGGRTADYKDD |
| | DDKGGGSG |
| MMP2/9 scramble control | LEGGGSGLALGPGGRTADYKDDD |
| | DKGGGSG |

Protease linker name and peptide sequence listed. Linkers contain Gly-Ser flexible sequences N- and C-terminal to the specific sequence (colored and underlined). Red down arrow indicated specific peptide bond hydrolyzed by the corresponding protease. Note that the MMP2/9 and MMP2/9-scramble linkers contain an enterokinase (flag tag) protease substrate to serve as a control.

### ProNotch Receptor Cells can Sense Endogenous Proteolytic Activities

So far, inventors have developed a protease activated receptor, ProNotch, that can be activated by supplementing a defined protease to the media. Inventors have demonstrated that the protease substrate linker can be exchanged to activate distinct receptors and induced complex phenotypic change such as myogenic differentiation. One of their goals in this project was to develop a system that could be activated by endogenous levels of protease expression by cells, which had yet to be realized. This would expand the utility of the ProNotch system for applications in sensing and responding to proteolysis in natural systems.

To evaluate the ProNotch receptor's ability to sense proteolytic activity expressed in cells as opposed to supplemented to media, inventors engineered cells to express secreted and surface tethered versions of TEVp shown to be functional in the secretory pathway¹⁸⁶. Inventors evaluated these protease ‘sender’ cells' abilities to trans-activate TEVs-ProNotch cells in coculture. In contrast to inventors expectations, tests involving the secreted or surface tethered versions of TEVp did not trans-activate TEVs-ProNotch cells. Furthermore, applying concentrated conditioned media from secreted TEVp-expressing cells to TEVs-ProNotch cells did not induce signaling. These results were surprising, as TEVs-ProNotch cells co-expressing these TEVp variants in cis induced signaling (data not shown). These results indicated that either the concentration of the TEVp was too low for detectable signal transduction in trans as opposed to in cis, or that the TEVp variant became nonfunctional in the extracellular space in our culture conditions. It is known that the native TEVp can autocatalytically cleave itself which reduces its activity¹⁸⁷. Future work in developing an orthogonal secreted protease to function with an accompanying ProNotch would complement the other synthetic signaling methods in the mammalian synthetic biology toolkit and should be further investigated.

Given cell-expressed TEVp did not trans-activate TEVs-ProNotch receptor cells, inventors turned their focus on detecting an endogenous protease expressed in a cancer cell line as a demonstration of cell-induced trans ProNotch activation. Inventors evaluated the protease substrate ‘MMP2/9’¹⁸⁸ that is cleaved by the gelatinase family of matrix metalloproteases¹⁸⁹: matrix metalloprotease-2 and matrix metalloprotease-9. These proteases are also known as gelatinase A or 72 kDa type IV collagenase, and gelatinase B or 92 kDa type IV collagenase, respectively. MMP2 and MMP9 play critical roles in extracellular degradation and regulate various physiological and pathological states including embryonic development, angiogenesis, inflammation and tumor progression. Like most extracellular proteases expressed in animals, MMP2 and MMP9 are produced as latent zymogens which require proteolytic processing of their N-terminal region (known as inhibitory or ‘pro’ domains) to gain catalytic activity. Gelatinase activity is further controlled by a family of specific endogenous tissue inhibitors of MMPs known as TIMPs (TIMP1-4 in humans), that bind reversibly to the MMP active site in a 1:1 stoichiometric ratio. The balance between MMPs and TIMPs is largely responsible for the control of ECM protein degradation. Due to this complex regulation at the post-translational level, MMP2 and MMP9 gene expression does not necessarily equate to activity. Furthermore, gelatin zymography, a technique commonly used in the literature to report gelatinase activity, can only indicate the relative presence of MMPs and TIMPs, but not the actual activity within the tissue¹⁹⁰.

Inventors chose gelatinases as initial targets because they are the best characterized proteases consistently detected in malignant tumors and have been shown to be expressed in several cancer cell lines, such as HT1080 fibrosarcoma cells. Inventors therefore designed ProNotch receptors with either the MMP2/9 substrate or scramble control linkers (Table 3).

### Pronotch Receptor Cells can Sense Endogenous Proteases Expressed in Cis

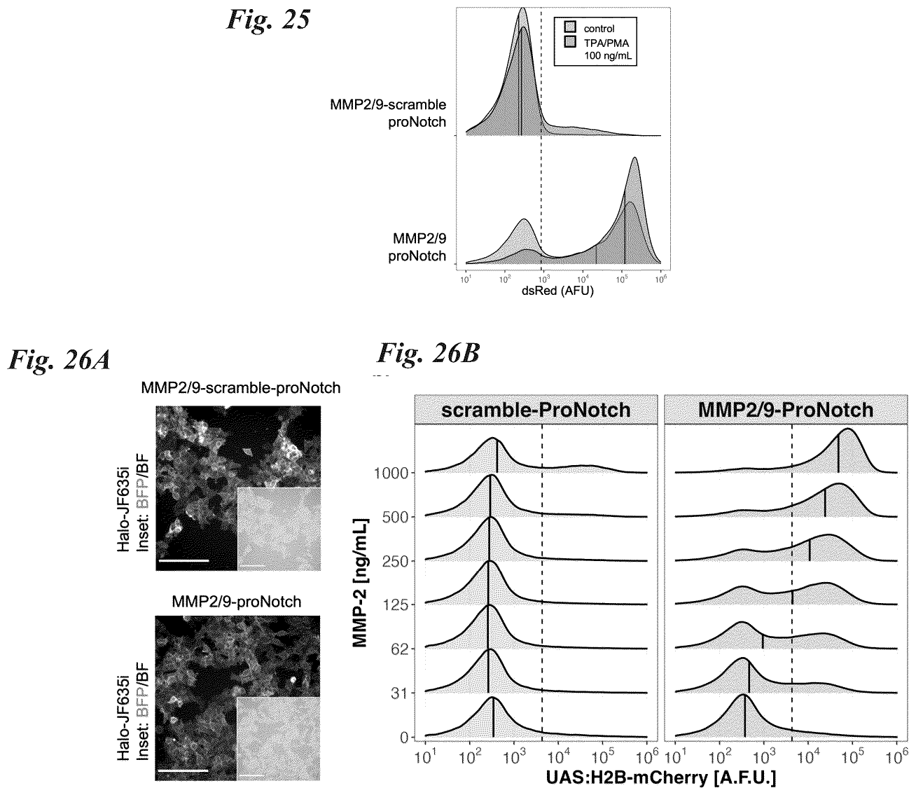

Interestingly, it was found that MMP2/9-ProNotch expression in inventors' U2OS reporter cells constitutively activated dsRed2 expression compared to the scramble control (FIG. 25). This is not surprising as it has been found that U2OS cells endogenously express both gelatinases¹⁹¹. Previous work by has shown that the pro- and active MMP9 levels can be increased in a dose-dependent manner via treatment with phorbol esters—in particular phorbol 12-myristate 13-acetate, also known as tetradecanoyl phorbol acetate. Inventors therefore evaluated PMA treatment to test if the MMP2/9-ProNotch activation in U2OS cells is sensitive to increases in MMP9. Consistent with expectation, PMA treatment of U2OS cells induced increased reporter activation in cells expressing MMP2/9-ProNotch, whereas cells expressing a scramble control-ProNotch exhibited comparable reporter gene activities under PMA treated and untreated conditions (FIG. 25). Therefore, MMP2/9-ProNotch is constitutively activated via endogenous U2OS gelatinase activities, and elevating MMP9 levels with PMA further increased reporter gene activity. These results indicate that ProNotch can facilitate the detection of endogenous proteases that are expressed in cis, and that their levels can be inferred from receptor-mediated reporter gene activities. Overall, this indicates ProNotch can serve as a tool for measuring proteolytic activity from cells and variation of their levels in response to biological stimuli.

Pronotch Receptor Cells are Activated in Trans Upon Stimulation with Protease-Secreting Cells

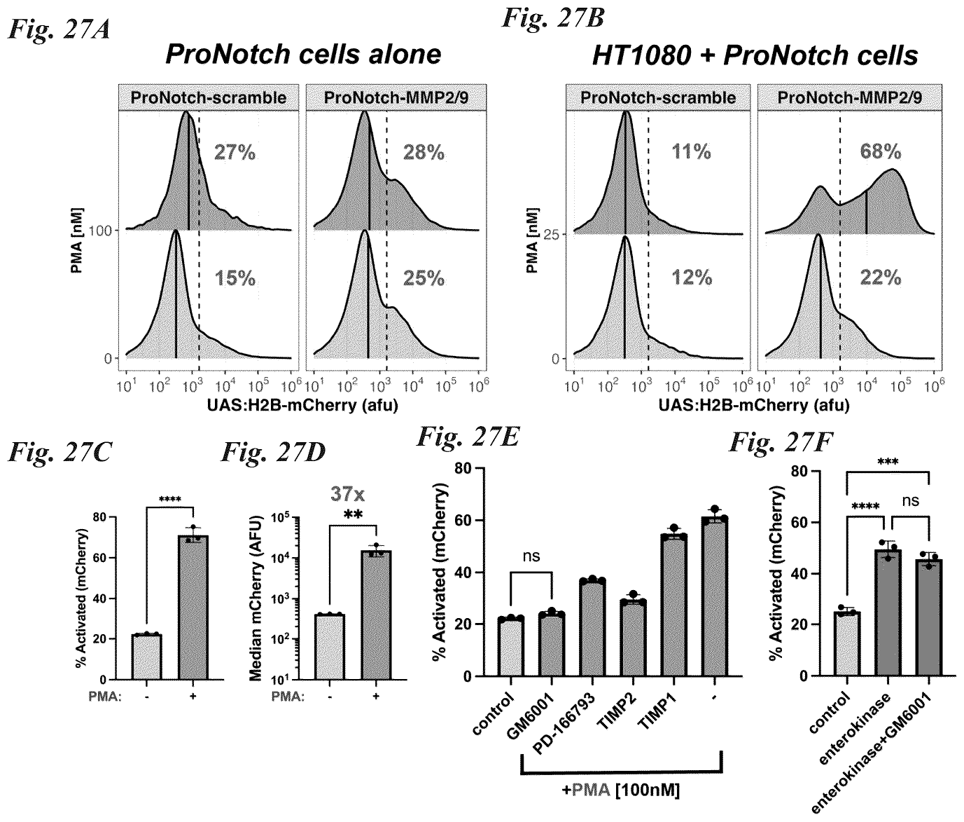

As U2OS cells already exhibit significant gelatinase activity based on reporter activation assays disclosed herein, inventors evaluated the MMP2/9-ProNotch in HEK293-FT reporter cells for trans protease sensing. Inventors carried out their analyses with clonal lines of the MMP2/9-ProNotch and scramble control receptor with comparable receptor surface expression and ‘T2A-BFP’ receptor co-expression fluorescent protein marker (FIG. 26A). HEK293 have been shown to endogenously express MMPs¹⁹², which could explain for the higher basal activation of the reporter gene observed between the ProNotch cell lines (bottom panel, FIG. 27A). This activation is however much lower than when MMP2/9-ProNotch was expressed in U2OS cells. Inventors first confirmed that exogenous gelatinase specifically activated the HEK-MMP2/9-ProNotch compared to the scramble control (FIG. 26B).

Next, inventors cocultured the ProNotch receptor cells with HT1080 fibrosarcoma cells, which naturally produce high levels of the inactive pro-forms of MMP2¹⁹³. HT1080 cells¹⁹⁴ were chosen as they are commonly used model system to study MMPs and their effect on 3D cell invasion and metastasis. PMA has been found to induce pro-MMP2 activation in HT1080 cells by shifting its activating protease—MMP14—from intracellular compartments to the cell surface^{195,196}. PMA additionally induces upregulation of MMP9 gene expression¹⁹³. When cocultured with HT1080 cells, both ProNotch cell line populations did not significantly activate compared to in monoculture, as expected (bottom panel, FIG. 27A-27B). This is due to the gelatinases produced by the HT1080 cells to be primarily in their inactive latent forms. When treating with PMA however, inventors observed HEK-MMP2/9-ProNotch activation in the HT1080 coculture, which was not observed in monoculture (top panel, FIG. 27A-27B). Differences in percent H2B-mCherry+ and median mCherry intensities with or without PMA treatment in the HEK-MMP2/9-ProNotch cells cocultured with HT1080 cells were found to be statistically significant (FIG. 27C-27D).

Inventors then evaluated if this activation was specific to gelatinase activity by testing various inhibitors. PMA cotreatment of cocultures with Ilomastat (GM6001), abroad spectrum MMP inhibitor, was found to inhibit HEK-MMP2/9-ProNotch activation to levels comparable to the control cocultures without PMA (FIG. 27E). Inventors confirmed that Ilomastat did not inhibit the ADAM cleavage of the ProNotch receptor by evaluating HEK293-MMP2/9-ProNotch activation with enterokinase with or without the inhibitor and did not observe a statistically difference in enterokinase-induced activation (FIG. 27F). This result suggests that an MMP is involved in the activation observed. Inventors additionally tested other inhibitors with higher specificity for MMP2 or MMP9 to determine if the activation is specific to gelatinase activity. Recombinant TIMP2 and the small molecule drug PD-166793 are specific inhibitors against MMP2, while TIMP1 is specific for MMP9¹⁸⁹. All of these molecules were found to decrease ProNotch signaling activities compared to treating the cocultures with PMA alone, but activation was higher than in the control cocultures (FIG. 27E). Collectively, these results suggest that both gelatinases are involved in the activation of HEK-MMP2/9-ProNotch cells in coculture with HT1080 cells. In summary, inventors demonstrated that ProNotch receptors can be developed to sense and respond to endogenously expressed proteases.

The Anti-NRR1 scFv-NRR Fusion can be Used as a Soluble Pro-Antibody for Targeted and Protease-Dependent Inhibition of Endogenous Notch Signaling

Throughout the development of the ProNotch receptor, inventors additionally recognized that an anti-NRR1 antibody-NRR fusion could potentially be used as a soluble protease-activated antibody (pro-antibody for inhibited Notch signaling. In this application, the NRR fusion serves as the inhibitory mask to the antibody fragment, thus preventing its ability to bind Notch in the intact ‘pro-antibody’ form. Once proteolytically processed however, the antibody and its fused NRR antigen is expected to dissociate, rendering the antibody fragment available to bind and antagonize Notch receptors.

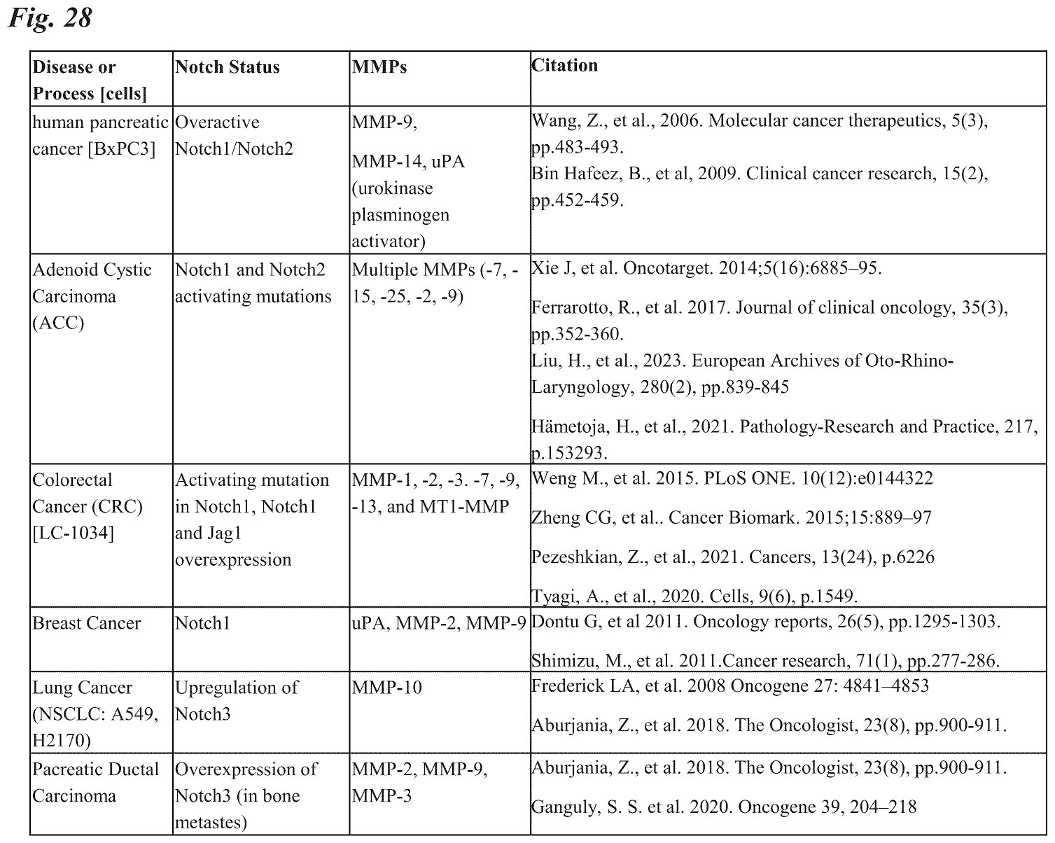

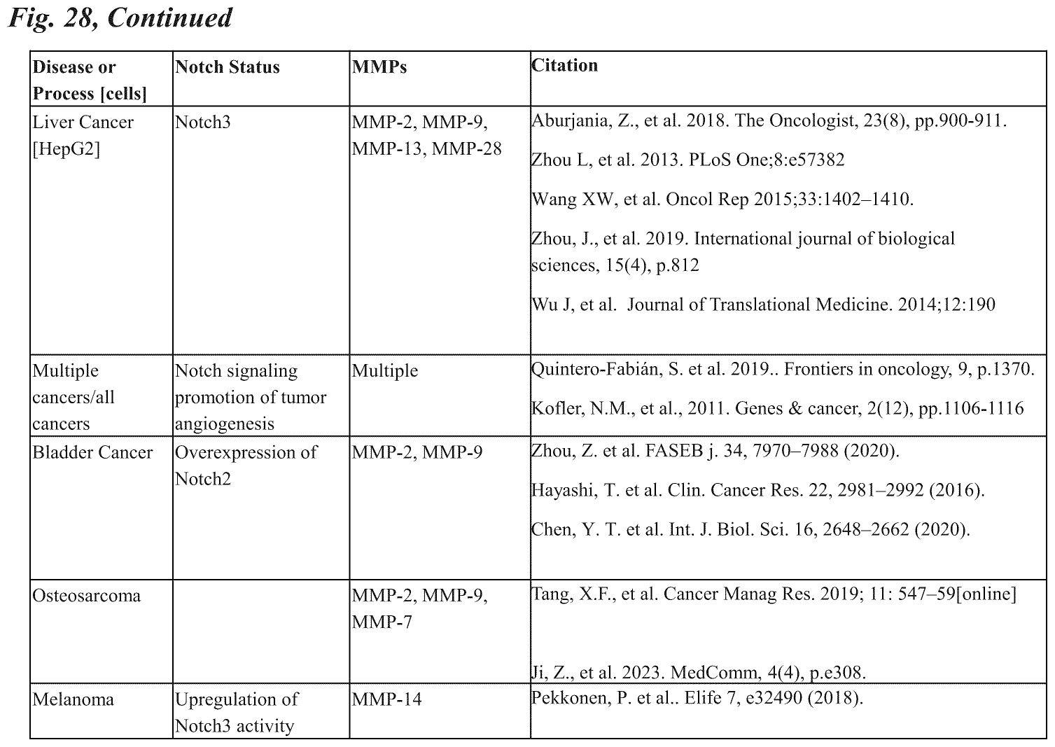

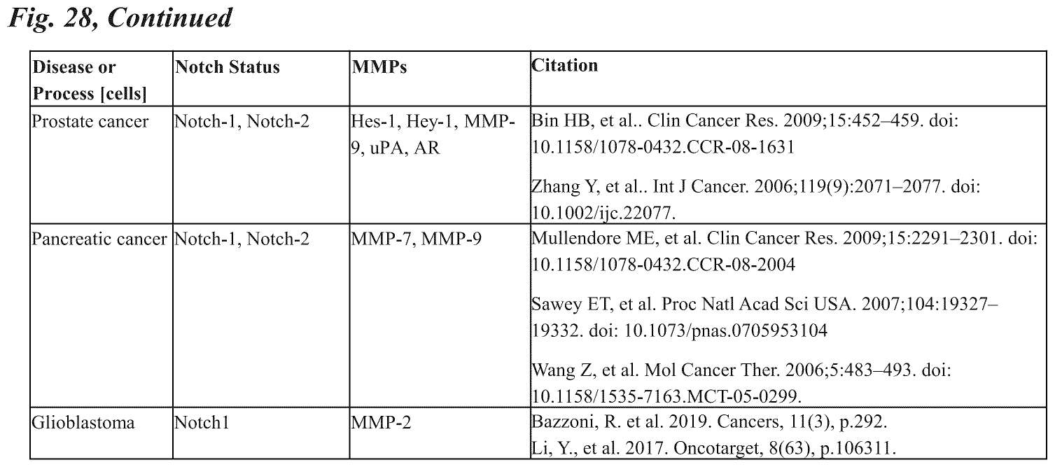

The motivation for this aspect of the work is that most pan-Notch inhibitors have failed in clinical trials due to dose-limiting toxicities as Notch signaling regulates diverse aspects of tissue homeostasis in many contexts¹. In clinical trials, patients treated with pan-Notch inhibitors—such as gamma secretase inhibitors—experience vascular defects, hepatoxicity, and severe gastrointestinal toxicities which have hindered the clinical utility of such therapies in clinical development^{197,198}. Overall, there is an urgent and unmet need for inhibitors that selectively block pathogenic Notch activity while allowing normal signaling in healthy tissues to proceed. The development of protease activated Notch signaling inhibitors could potentially contribute to address this gap, since aberrant proteolysis is implicated in several diseases that are driven by Notch signaling, such as cancer (FIG. 28). MMPs, particularly MMP2, MMP9 and MMP14 are frequently overexpressed in the tumor microenvironment and are associated with invasive behavior and metastatic potential. In contrast, the expression and activities of these enzymes are tightly regulated with minimal ECM degradation in healthy tissues. This differential activity provides a unique opportunity to exploit MMPs as tumor-selective triggers for therapeutic activation. By designing inhibitors that are conditionally activated in the presence of elevated MMP activity, inventors aim to target malignant cells precisely while sparing normal tissues from off-target effects.

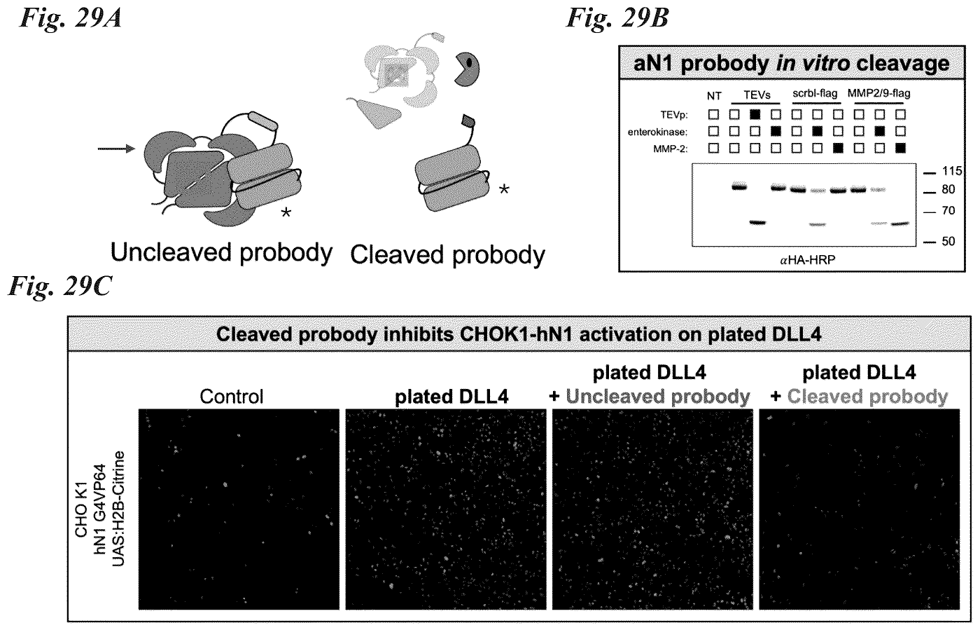

As a model construct to test the feasibility of our pro-antibody approach, inventors generated a secreted pro-antibody of the anti-NRR1 scFv fused to murine NRR(V1677D) with an MMP2/9 linker. Similar to our receptor-based analyses, the NRR(V1677D) domain would bind (and thus mask) the anti-NRR1 scFv in the construct's inactive state. A HaloTag was additionally added to label this molecule with a dye (Halo-JF635i). To acquire the pro-antibody, inventors transfected HEK293-FT cells with a plasmid encoding the secreted fusion and harvested conditioned media. Inventors first demonstrated that the soluble forms of the fusion can be processed in vitro. Like the ProNotch receptor results, proteolysis was specific to the substrate linker used in several designs (FIG. 29B). Inventors then examined the Notch-binding and signaling-antagonizing behaviors of the construct in its cleaved and uncleaved form.

As a first test, inventors examined whether the generated pro-antibody could bind NOTCH1 in a protease-dependent manner. Inventors treated CHO-K1 cells expressing a stably integrated NOTCH1 receptor with the TEVp-cleaved and uncleaved forms of the pro-antibody conjugated to Halo-JF635i. Consistent with their expectations, inventors observed binding of only the proteolytically processed pro-antibody (data not shown). Next, inventors evaluated signaling responses using a NOTCH1 chimera expressing CHO-K1 cells with a signaling-dependent reporter gene (NOTCH1 ECD-GAL4 ICD, UAS:H2B-Citrine). Because signaling in these cells is quiescent in the absence of ligand, inventors stimulated signaling by growing the cells on immobilized Notch ligand (DLL4-Fc). As expected, reporter activity was strongly induced in pro-antibody treated cells under TEVp-lacking conditions, whereas signaling levels were strongly attenuated in cells co-incubated with TEVp (FIG. 29C). Together with binding analyses disclosed herein, these data show that the pro-antibody can conditionally bind and inhibit NOTCH1 in the presence of its corresponding activating protease (in this case TEVp).

In these preliminary tests inventors used the NRR variant with the V1677D mutation as the inhibitory mask, with the idea that once processed the NRR spontaneously disassembles. This ‘triggered’ disassembly of the inhibitory domain would make the cleaved, unbound NRR domains no longer competitive inhibitors for the activated anti-NRR1 antagonists. This could potentially be a mechanism to enable lower effective doses for signaling inhibition.

Overall, inventors demonstrate the feasibility of conditionally inhibiting Notch by proteolysis. Further characterization and validation are required to determine if these molecules could potentially function in therapeutic applications. This includes inhibiting ligand-independent and natural Notch transactivation between cells, testing this design in a full antibody construct, and determining the dose-response of these molecules. Inventors anticipate herein that this strategy could readily be extended to render Notch2 and Notch3 antagonizing antibodies conditionally activated by proteolysis.

### Example 2

### Tumor-Targeted Antibody-Based Inhibitors of Oncogenic Mutant Notch Receptors

Inventors generate tumor-selective Notch inhibitors by engineering antibodies that can bind and block Notch receptors in an MMP-cleavage-dependent way. To create such inhibitors, inventors generate MMP-activated “pro-antibodies,” antibody-based ‘prodrugs’ linked to ‘masking sequences’ that prevent their binding to target antigens within healthy tissues. Using MMP-cleavable linkers to mask Notch1-inhibiting antibodies conditionally, inventors generate environmentally-specific inhibitors that bind and block Notch receptors only within tumor-containing overexpressed MMPs. These agents are able to inhibit pathogenic Notch activity selectively within tumor sites, including that of multiple mutant Notch forms. Experiments disclosed herein will overcome the dose-limiting toxicities that have stymied existing small molecule and antibody-based Notch inhibitors, thereby enabling the design of new agents to address the unmet need for selective Notch inhibition in cancer treatment.

The Notch signaling pathway is essential for cell differentiation, proliferation, and tissue survival. However, dysregulated Notch activity is linked to many cancers. Current therapies targeting Notch face limitations due to significant toxicities from pan-Notch inhibition, including gastrointestinal and cardiovascular side effects. These adverse effects limit the clinical utility of Notch inhibitors, creating an unmet need for more selective approaches.

The invention disclosed herein develops tissue-targeted, antibody-based Notch inhibitors that selectively block oncogenic Notch signaling in diseased tissues while preserving normal signaling in healthy areas. The inventors' approach focuses on next-generation Notch inhibitors that block specific Notch isoforms (Notch-1, -2, or -3) in a tissue-targeted manner, reducing systemic toxicities associated with pan-Notch inhibition. These antibody-based inhibitors are designed to localize therapeutic action to diseased sites, minimizing effects on normal tissues and enhancing the safety and efficacy of Notch-targeting therapies.

The invention disclosed herein transforms cancer treatment driven by dysregulated Notch signaling by creating a platform for developing tissue-selective inhibitors. These inhibitors allow more effective, safer treatments and open new possibilities for treating diseases where Notch inhibition was previously too risky. Additionally, these agents are adapted as antibody-drug conjugates to achieve dual signaling inhibition and targeted cytotoxicity.

The Notch pathway regulates cell fate, differentiation, and proliferation. Mutant Notch receptors, particularly Notch1, Notch2, and Notch3, are oncogenic drivers in T-cell acute lymphoblastic leukemia (T-ALL), adenoid cystic carcinoma, and breast cancer, among other. Mutations typically fall into two categories: (1) Gain-of-Function (GOF) mutations lead to ligand-independent receptor activation, common in T-ALL; (2) Ligand-sensitizing mutations heighten sensitivity to ligands, resulting in excessive pathway activation. These mutations make mutant Notch receptors prime therapeutic targets for selective inhibition, potentially halting cancer progression while sparing normal tissue function.

Preclinical studies have shown that cancer cell lines with Notch mutations depend on Notch signaling for survival. Targeted inhibition via genetic or pharmacologic methods (e.g., CRISPR, gamma-secretase inhibitors) has reduced proliferation and increased apoptosis in mutant Notch cancer cells, with lower toxicity in normal cells, supporting tissue-selective approaches. (Aster et al., doi: 10.1016/j.cell.2017.09.034.)

in vivo models such as patient-derived xenografts and genetically engineered mice, have shown significant tumor regression upon Notch inhibition, particularly when mutations are tissue-specific. These findings suggest that targeted inhibition can reduce toxicity compared to pan-Notch approaches. (Wilson and Radtke, doi:10.1016/j.febslet.2006.04.039)

Clinical evidence from trials of Notch inhibitors, including gamma-secretase inhibitors and monoclonal antibodies, supports their potential but highlights challenges with adverse effects. Biomarker analyses reinforce Notch mutations as predictive markers of response, validating mutant Notch receptors as actionable targets. (Takebe et al. doi:10.1016/j.pharmthera.2013.09.005) The Notch-targeted therapy field includes several approaches:

Gamma-Secretase Inhibitors (GSIs): Block the release of the Notch intracellular domain, inhibiting pathway activation. Although multiple small molecule GSI have progressed to clinical trials, two significant drawbacks of these compounds have curtailed their therapeutic utilizations: (a) their inability to distinguish between individual Notch isoforms (including mutant and wild-type Notch forms), and (b) their interferences with the transmembrane cleavage of other GS cleavage substrates, of which over 120 substrates in the human proteome have been identified.

Notch Transcription Complex Inhibitors: Small molecules such as CB-103 (developed by Cellestia Biotech) inhibit Notch transcription complex formation by acting intracellularly to block interactions between relevant protein domains. CB-103 is being evaluated in clinical studies and early results report reduced gastrointestinal toxicities. However, like GSIs, CB-103 is similarly limited by its pan-blockage of both normal and aberrant Notch activities.

Monoclonal Antibodies: In contrast to small molecule blockers, antibody-based inhibitors provide a means to block specific Notch isoforms and specific Notch ligands selectively. Antibodies targeting specific Notch receptors or ligands (e.g., Brontictuzumab for Notch1 and Demcizumab for DLL4) have been developed and evaluated in clinical trials. While these agents offer greater specificity compared to GSI and CB-103, clinical data show that toxicity challenges remain (attributable to pan-Notch1 inhibition for Brontictuzumab, and pan-DLL4 blockade for Demcizumab).

The invention disclosed herein will generate tumor-selective Notch inhibitors by engineering antibodies that can bind and block Notch receptors in a matrix metalloproteinase (MMP)-cleavage-dependent way. To create such inhibitors, inventors generate MMP-activated “pro-antibodies,” which are antibody-based ‘prodrugs’ linked to ‘masking sequences’ to prevent their binding to antigens within healthy tissues. By using MMP-cleavable linkers to mask Notch1-inhibiting antibodies conditionally, inventors generate selectively Notch inhibitors that can bind and block Notch receptors only upon MMP-mediated linker cleavage.

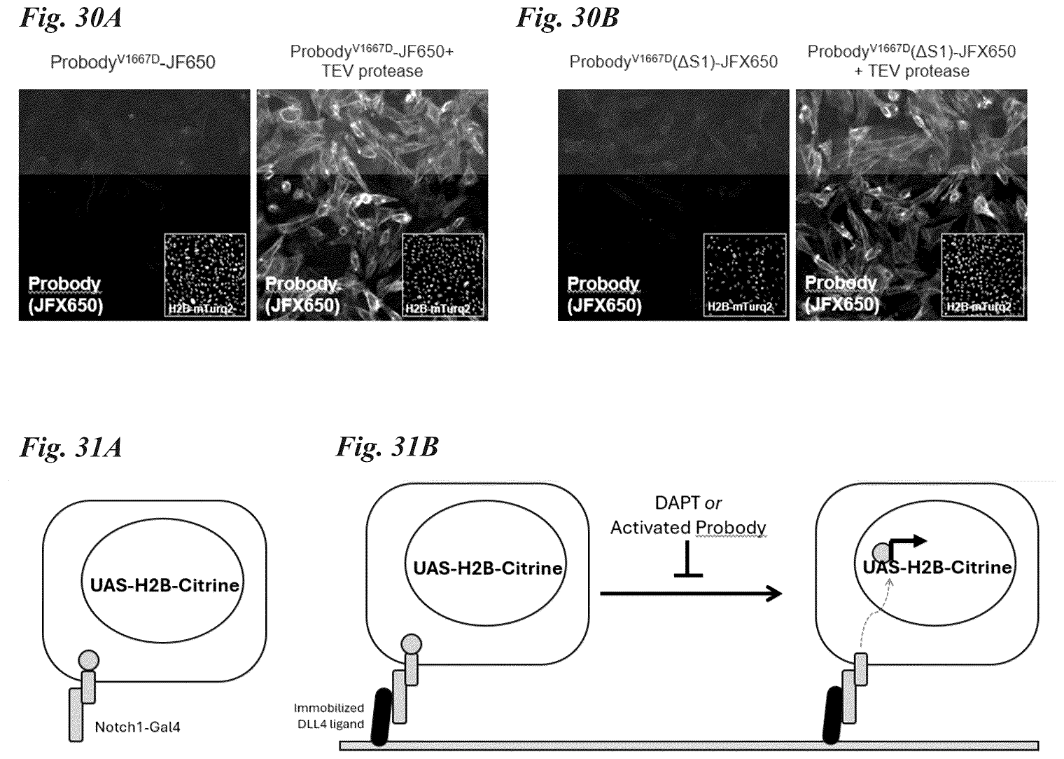

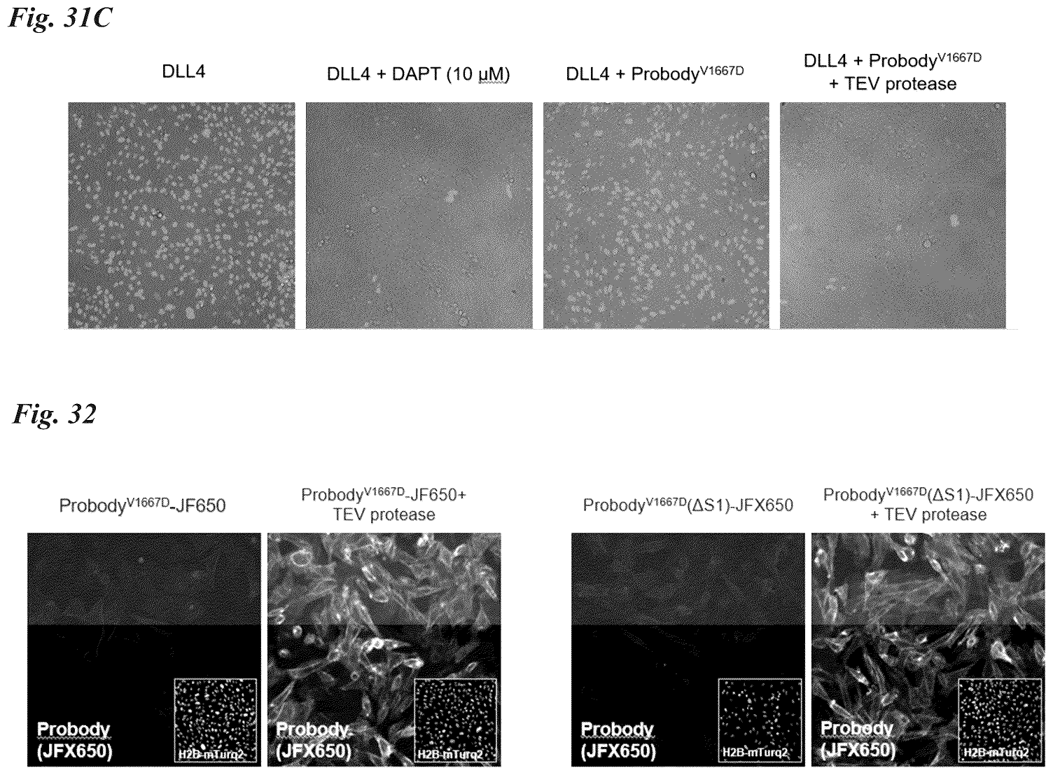

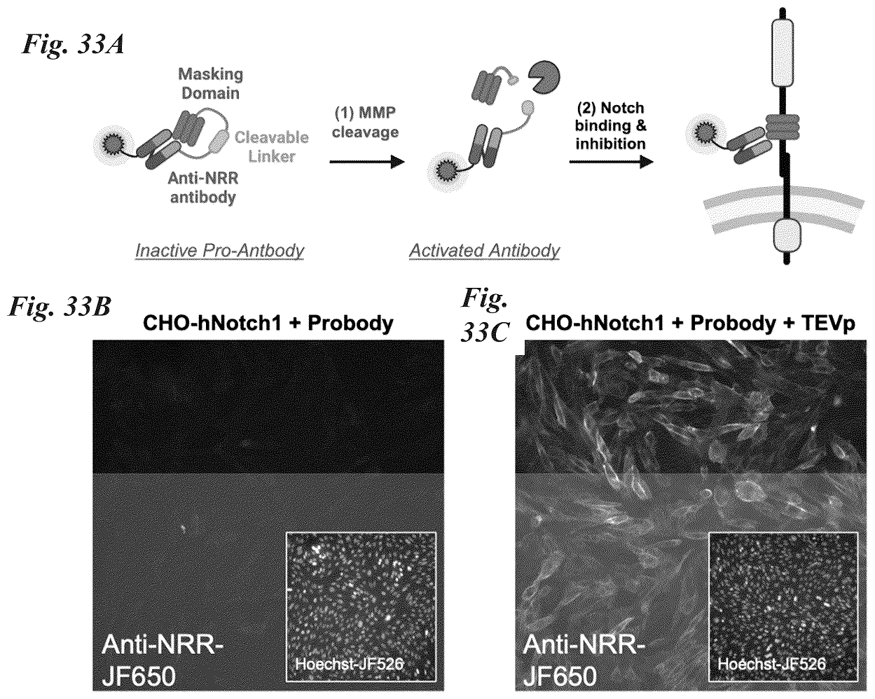

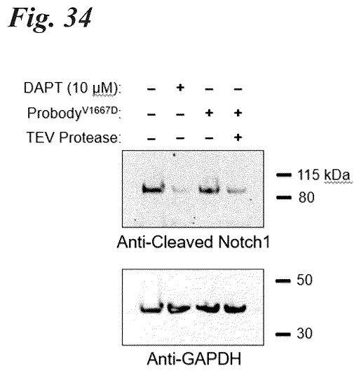

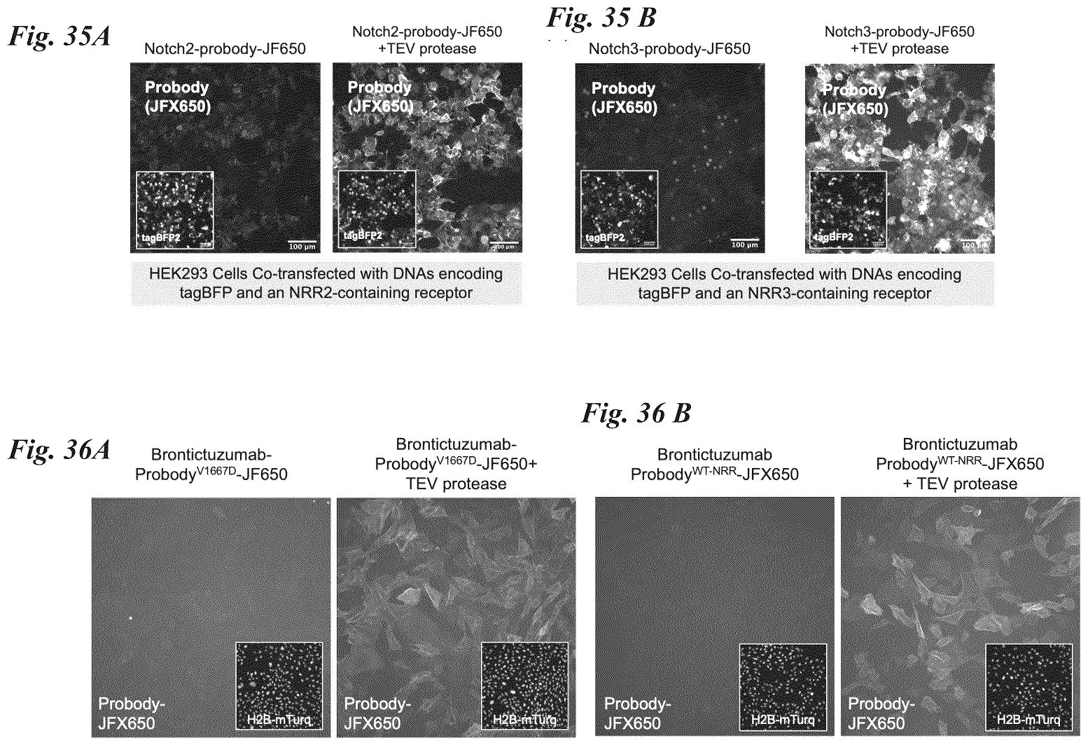

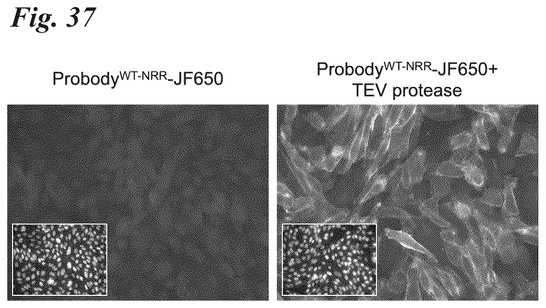

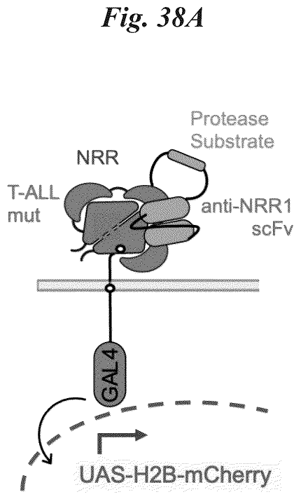

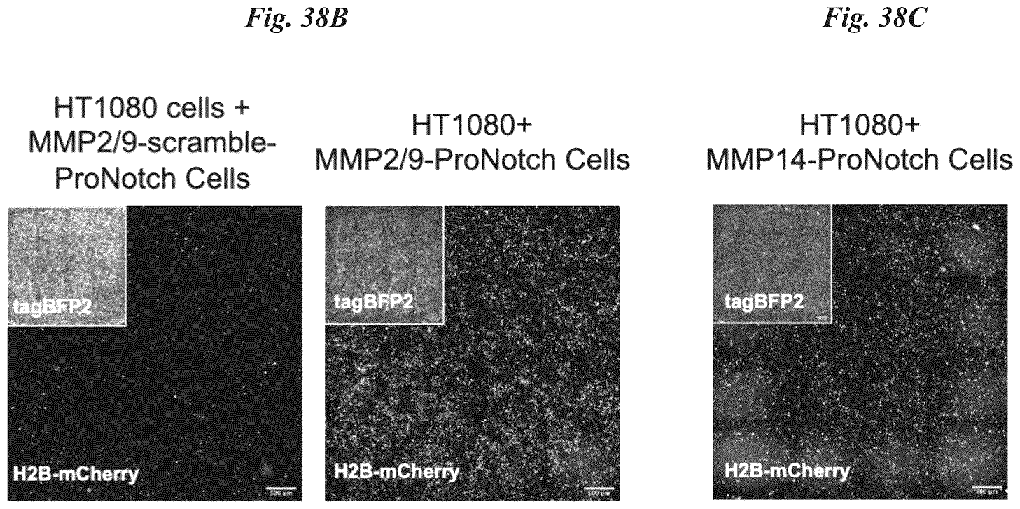

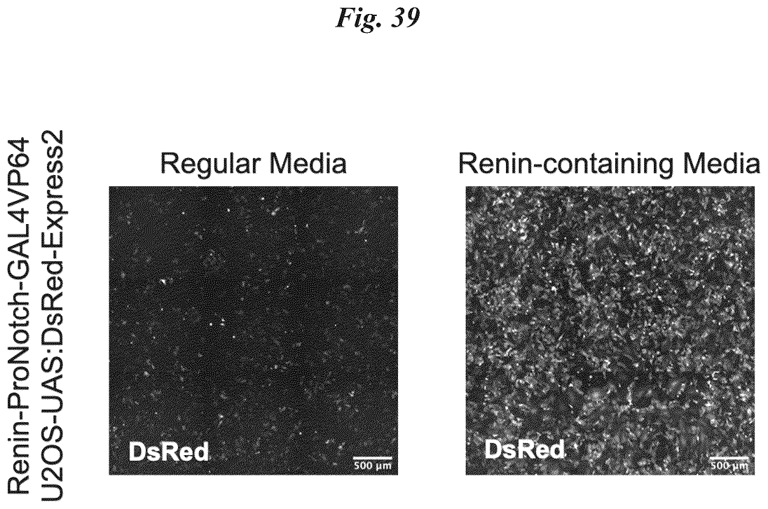

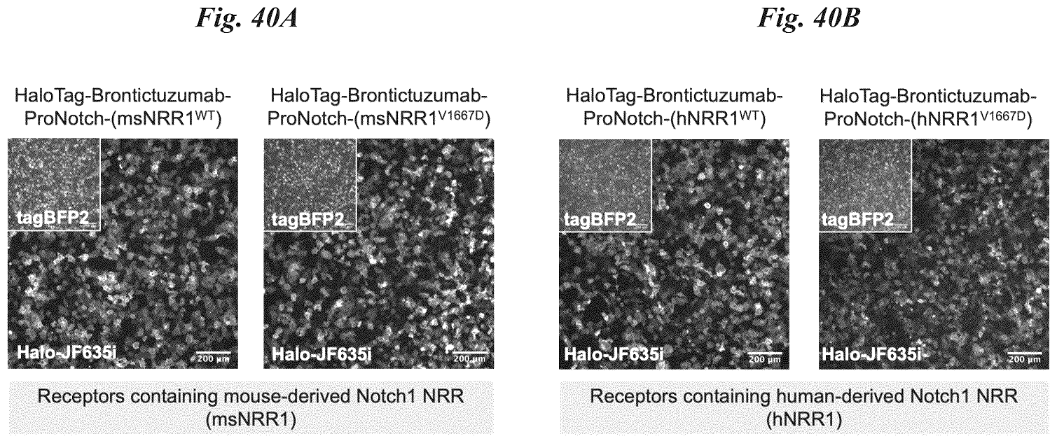

Key to designing a useful pro-antibody is identifying a suitable masking sequence. Requirements of such a mask include (1) the ability to block antigen binding in the uncleaved state efficiently and (2) the ability to unbind the immunoglobulin upon linker cleavage rapidly. Inventors have developed a masking sequence that satisfies these requirements, and have used their mask to design pro-antibody-based signaling inhibitors against Notch1 (FIGS. 30A-3). Initial tests show that the pro-antibody can inhibit GOF Notch mutant signaling activity in an MMP-dependent manner. Given that MMPs are known to be overexpressed in numerous tumor types, we predict that these pro-antibodies will be able to inhibit pathogenic Notch1 activity selectively within tumor microenvironments.

Antibodies against the Negative Regulatory Regions (NRRs) of Notch1, Notch2, and Notch3 are used to design protease-dependent signaling inhibitors. Sequences that are activated by tumor-associated MMPs are generated and validated.

Inventors evaluate the activity and specificity of protease-activated Notch inhibitors in signaling assays and against Notch-dependent cancer cell lines, including MMP-expressing cancer lines bearing Notch NRR mutations.

1. A synthetic nucleic acid encoding a mutant Notch NRR (Negative Regulatory Region), wherein the mutant Notch NRR comprises a mutation that induces ligand-independent activation of regulated intramembrane proteolysis resulting to Notch signaling, SynNotch signaling, or at least another signaling outcome.

2. The synthetic nucleic acid of claim 1, wherein the mutant Notch NRR comprises at least point mutation as compared to a wild-type Notch NRR.

3. The synthetic nucleic acid of claim 1, wherein the mutant Notch NRR comprises a V1667D mutation as compared to a wild-type Notch NRR, or wherein the mutant Notch NRR comprises a mutation selected from Table 4.

4. The synthetic nucleic acid of claim 1, wherein the mutant Notch NRR sequence encodes a protein sequence of SEQ ID NO: 1 or 2.

5. A fusion protein comprising:
i) a Notch NRR (Negative Regulatory Region)-binding antibody, and
ii) a mutant Notch NRR comprising a mutation that induces ligand-independent activation of regulated intramembrane proteolysis resulting to Notch signaling, SynNotch signaling, or at least another signaling outcome.

6. The fusion protein of claim 5, wherein the Notch NRR-binding antibody inhibits the ligand-independent activation of Notch signaling by the mutant Notch NRR.

7. The fusion protein of claim 5, wherein the Notch NRR-binding antibody is a Notch NRR-binding scFv, Notch NRR-binding nanobody, or Notch NRR-binding scFab.

8. The fusion protein of claim 5, wherein the mutant Notch NRR comprises at least at least one of:
at least one point mutation as compared to a wild-type Notch NRR (SEQ ID NO: 3 or 4);
a V1667D mutation as compared to a wild-type Notch NRR;
a mutation disclosed in Table 4; and
a sequence of SEQ ID NO: 1 or 2.

9. The fusion protein of claim 5, wherein the Notch NRR-binding antibody is at least one of:
further fused to a transmembrane domain;
further comprises, positioned in between i) and ii), at least one linker;
further comprises at least one linker; and
further comprises at least one linker positioned between the heavy and light chains.

10. The fusion protein of claim 9, wherein the at least one linker is a cleavable linker.

11. The fusion protein of claim 10, wherein the cleavable linker is cleaved by a protease, an enzyme, chemical reagent, light, pH, or ultrasound.

12. The fusion protein of claim 11, wherein the at least one linker is cleaved by a protease selected from the group consisting of: Matrix Metalloproteinases (MMPs), Urokinase, aka urokinase-type plasminogen activator (uPA), Tobacco Etch Virus (TEV) protease, Enterokinase (enteropeptidase), Prostate-Specific Antigen (PSA), Cathepsins, ADAM-family proteases, ADAM-TSs, Kallikreins, Legumain, Fibroblast activating protein-α (FAP), Renin, Human Rhinovirus (HRV) 3C protease, Bacterial sortases, Thrombin, and Type II Transmembrane Serine Proteases.

13. The fusion protein of claim 7, wherein the Notch NRR-binding scFv comprises at least one of
from N-terminal to C-terminal and in covalent linkage, a V_{H} domain, a linker domain, and a V_{L} domain; and
a sequence selected from any one of SEQ ID NOs: 7-12.

14. The fusion protein of claim 5, wherein the fusion protein further comprising a signal sequence at its N-terminal.

15. A synthetic Notch receptor protein comprising, from N-terminal to C-terminal and in covalent linkage, (i) a scFv that binds to an at least one Notch NRR, (ii) at least one linker, (iii) a mutated Notch NRR, wherein the mutant Notch NRR comprises a mutation that induces ligand-independent activation of regulated intramembrane proteolysis resulting to Notch signaling, SynNotch signaling, or at least another signaling outcome, (iv) a transmembrane domain, and (v) an intracellular domain.

16. A protease regulatable Notch signaling modulator, the modulator comprising an anti-NRR antibody fused to a mutant NRR, having at least one protease cleavable linker positioned between the anti-NRR immunoglobulin element and mutant NRR.

17. The protease regulatable Notch signaling modulator of claim 16, wherein the anti-NRR antibody fused further fused to a transmembrane domain.

18. The protease regulatable Notch signaling modulator of claim 16, wherein the anti-NRR immunoglobulin element is a NRR-binding agonist capable of activating or inhibiting Notch receptors upon cleavage and separation from the fused mutant NRR.

19. The protease regulatable Notch signaling modulator of claim 16, wherein modulation is an increase in Notch signaling.

20. The protease regulatable Notch signaling modulator of claim 16, wherein modulation is a decrease in Notch signaling.

21. The protease regulatable Notch signaling modulator of claim 16, the modulator is further conjugated to a drug, forming an antibody-drug conjugate; or wherein the modulator is used to deliver cellular cargo.
